(19) United States
(12) Patent Application Publication
(10) Pub. No.: US 2020/0237766 A1
(43) Pub. Date: Jul. 30, 2020

(54) PKM2 ACTIVATORS IN COMBINATION WITH REACTIVE OXYGEN SPECIES FOR TREATMENT OF CANCER

(71) Applicant: TOLERO PHARMACEUTICALS, INC., Lehi, UT (US)

(72) Inventors: Adam SIDDIQUI-JAIN, South Jordan, UT (US); Peter W. PETERSON, Salt Lake City, UT (US); Clifford J. WHATCOTT, West Jordan, UT (US); David J. BEARSS, Alpine, UT (US); Steven L. WARNER, Sandy, UT (US)

(21) Appl. No.: 16/755,868

(22) PCT Filed: Oct. 12, 2018
(86) PCT No.: PCT/US2018/055668

§ 371 Date: Apr. 13, 2020

Related U.S. Application Data
(60) Provisional application No. 62/572,237, filed on Oct. 13, 2017.

(51) Int. Cl. A61K 31/519; A61K 31/4155; A61K 31/496; A61K 31/704; A61K 31/4965; A61K 31/44; A61K 31/395; A61P 35/00

(52) U.S. Cl. CPC A61K 31/519; A61K 31/4155; A61K 31/496; A61P 35/00; A61K 31/4965; A61K 31/44; A61K 31/395; A61K 31/704

(57) ABSTRACT

Combination therapies for treatment of cancer are provided. The disclosed methods comprise administration of a PKM2 activator and an anti-cancer drug having a mechanism of action that increases production of reactive oxygen species in cancer cells to a patient in need thereof.

### BACKGROUND

### Field

Embodiments of the present invention are generally directed to methods for treatment of cancer by administration of a PKM2 activator and an anti-cancer drug having a mechanism of action that increases production of reactive oxygen species in cancer cells.

### Description of the Related Art

Glucose provides cancer cells with building blocks in the form of glycolytic pathway intermediates (Mazurek S., Int. J. Biochem. Cell. Biol. 43(7):969-80 (2010); Vander Heiden M. G., Cantley L. C., Thompson C. B., Science 324(5930):1029-33 (2009)). The main enzyme regulating flux through the glycolytic pathway in cancer cells, and thus the level of available intermediates, is the M2 splice form of pyruvate kinase (PKM2), which controls the rate-limiting final step in glycolysis. PKM2 is upregulated in cancer cells (Altenberg B., Greulich K. O., Genomics 84(6):1014-20 (2004)), and has been shown to increase tumorigenicity compared to the alternatively spliced and constitutively active PKM1 isoform (Christofk H. R., Vander Heiden M. G., Harris M. H., et al., Nature 452(7184):230-33 (2008); Goldberg M. S., Sharp P. A., J. Exp. Med. 209(2):217-24 (2012)).

Past efforts have focused on the discovery and development of small molecule PKM2 activators (Boxer M. B., Jiang J. K., Vander Heiden M. G., et al., J. Med. Chem. 53(3):1048-55 (2010); Jiang J. K., Boxer M. B., Vander Heiden M. G., et al., Bioorg. Med. Chem. Lett. 20(11):3387-93 (2010); Walsh M. J., Brimacombe K. R., Veith H., et al., Bioorg. Med. Chem. Lett. 21(21):6322-27 (2011)) as a potentially useful anti-cancer therapy for treatment of sarcoma, brain, colorectal, kidney, head and neck, lung, ovarian, pancreatic and prostate cancers.

Alternatively, drugs producing reactive oxygen species (ROS), such as anthracycline-based molecules, are widely used in chemotherapy regimens to treat multiple types of cancer. Although such ROS producing drugs are have been used extensively as cancer therapeutics, they are often associated with significant side effects, for example, cardiotoxicity from anthracyclines (Minotti, G. et al. Pharmacol. Rev., 2004, 56(2), 185-229), peripheral neuropathy from bortezomib (Richarson, P. G., et al. Leukemia, 2012, 26(4) 595-608), and hand-foot syndrome from sorafenib (Chu, D., et al. Acta. Oncol. 2008, 47, 176-186). As such, a need exists for improving the efficacy and therapeutic index of ROS-producing anti-cancer drugs.

To date, there have been no reports of combinations of PKM2 activators and ROS-producing drugs. Thus, there remains a need for improved combination therapies for treatment of various cancers. Embodiments of the present invention fulfill these needs and provide related advantages.

### BRIEF SUMMARY

In brief, embodiments of the present invention provide methods for treatment of cancer comprising administration of two different therapeutic agents. For example, administration of an agent to decrease glutathione levels in cancer cells, such as a PMK2 activator, and an anti-cancer drug having a mechanism of action that increases ROS-production. In one embodiment, the disclosure provides a method for treating cancer in a patient in need thereof, the method comprising administering to the patient a therapeutically effective amount of the following therapeutic agents:

i) a PKM2 activator; and

ii) an anti-cancer drug having a mechanism of action that increases production of reactive oxygen species (ROS) in cancer cells upon administration to the patient.

Kits comprising a PKM2 activator, an anti-cancer drug having a mechanism of action that increases production of ROS in cancer cells upon administration to a patient, and instructions for administering the PKM2 activator and the anti-cancer drug to a patient in need of treatment of cancer, as well as pharmaceutical compositions useful in the disclosed methods, are also provided.

These and other aspects of embodiments of the invention will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE DRAWINGS

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements, and have been solely selected for ease of recognition in the figures.

FIGS. 1A-F show comparisons of cell viability when treated with anthracycline or anthracenedione compounds in combination with Compound 91.

FIG. 2 is cell viability plotted against concentration for an HSP90 inhibitor alone and in combination with a representative PKM2 activator.

FIG. 3 presents data showing the effect of combining Sorafenib and a representative PKM2 activator.

FIGS. 4A-B show data illustrating the synergistic combination of Bortezamib and representative PKM2 activators.

FIGS. 5A-C demonstrate the lack of synergy when PKM2 activators are combined with non-ROS producing drugs.

FIGS. 6A-B provide data showing decreased glutathione levels in cells treated with representative PKM2 activators.

FIGS. 7A-B show xenograph data indicating the synergistic effect of treating tumors with a combination of doxorubicin and a representative PKM2 activator.

FIG. 8 compares tumor growth curves (mean tumor volume over time) of different groups treated with various combinations of doxorubicin and Compound 91.

FIG. 9 shows the tumor growth curve of mice in Group 1 (received vehicle, 0 mg/kg, QD×3 weeks, p.o., saline, 0 mg/kg, Q2D×3 weeks, i.v.).

FIG. 10 shows the tumor growth curve of mice in Group 2 (received vehicle 0 mg/kg, QD×3 weeks, p.o., doxorubicin, 2 mg/kg, Q2D×3 weeks, i.v.).

FIG. 11 shows the tumor growth curve of mice in Group 3 (received Compound 91, 100 mg/kg, QD×3 weeks, p.o., saline, 0 mg/kg, Q2D×3 weeks, i.v.).

FIG. 12 shows the tumor growth curve of mice in Group 4 (received Compound 91, 200 mg/kg, QD×3 weeks, p.o., saline, 0 mg/kg, Q2D×3 weeks, i.v.).

FIG. 13 shows the tumor growth curve of mice in Group 5 (received Cmpd 91, 100 mg/kg, QD×3 weeks, p.o., doxorubicin, 2 mg/kg, Q2D×3 weeks, i.v.).

FIG. 14 shows the tumor growth curve of mice in Group 6 (received doxorubicin, 2 mg/kg, Q2D×3 weeks, i.v., Cmpd 91, 200 mg/kg, QD×3 weeks, p.o.).

FIG. 15 shows the results of mean body weight changes in the tumor bearing mice treated with various combinations of doxorubicin and Compound 91.

FIG. 16 shows the results of individual body weight changes in Group 1 (received vehicle, 0 mg/kg, QD×3 weeks, p.o., saline, 0 mg/kg, Q2D×3 weeks, i.v.).

FIG. 17 shows the results of individual body weight changes in Group 2 (received vehicle 0 mg/kg, QD×3 weeks, p.o., doxorubicin, 2 mg/kg, Q2D×3 weeks, i.v.).

FIG. 18 shows the results of individual body weight changes in Group 3 (received Compound 91, 100 mg/kg, QD×3 weeks, p.o., saline, 0 mg/kg, Q2D×3 weeks, i.v.).

FIG. 19 shows the results of individual body weight changes in Group 4 (received Compound 91, 200 mg/kg, QD×3 weeks, p.o., saline, 0 mg/kg, Q2D×3 weeks, i.v.).

FIG. 20 shows the results of individual body weight changes in Group 5 (received Cmpd 91, 100 mg/kg, QD×3 weeks, p.o., doxorubicin, 2 mg/kg, Q2D×3 weeks, i.v.).

FIG. 21 shows the results of individual body weight changes in Group 6 (received doxorubicin, 2 mg/kg, Q2D×3 weeks, i.v., Cmpd 91, 200 mg/kg, QD×3 weeks, p.o.).

FIG. 22 shows photos of the Group 1 mice (received vehicle, 0 mg/kg, QD×3 weeks, p.o., saline, 0 mg/kg, Q2D×3 weeks, i.v.).

FIG. 23 shows photos of tumors removed from the mice of Group 1.

FIG. 24 shows photos of the Group 2 mice (received vehicle 0 mg/kg, QD×3 weeks, p.o., doxorubicin, 2 mg/kg, Q2D×3 weeks, i.v.).

FIG. 25 shows photos of tumors from Group 2.

FIG. 26 shows photos of the Group 3 mice (received Compound 91, 100 mg/kg, QD×3 weeks, p.o., saline, 0 mg/kg, Q2D×3 weeks, i.v.).

FIG. 27 shows photos of tumors from Group 3.

FIG. 28 shows photos of the Group 4 mice (received Compound 91, 200 mg/kg, QD×3 weeks, p.o., saline, 0 mg/kg, Q2D×3 weeks, i.v.).

FIG. 29 shows photos of tumors from Group 4.

FIG. 30 shows photos of the Group 5 mice (received Cmpd 91, 100 mg/kg, QD×3 weeks, p.o., doxorubicin, 2 mg/kg, Q2D×3 weeks, i.v.).

FIG. 31 shows photos of tumors from Group 5.

FIG. 32 shows photos of the Group 6 mice (received doxorubicin, 2 mg/kg, Q2D×3 weeks, i.v., Cmpd 91, 200 mg/kg, QD×3 weeks, p.o.).

FIG. 33 shows photos of tumors from Group 6.

### DETAILED DESCRIPTION

In the following description, certain specific details are set forth in order to provide a thorough understanding of various embodiments of the invention. However, one skilled in the art will understand that embodiments of the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the following terms have the following meanings:

“Amino” refers to the -NH₂ radical.

“Cyano” or “nitrile” refers to the —CN radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” refers to a straight or branched hydrocarbon chain radical consisting solely of carbon and hydrogen atoms, which is saturated or unsaturated (i.e., contains one or more double and/or triple bonds), having from one to twelve carbon atoms (C₁-C₁₂ alkyl), preferably one to eight carbon atoms (C₁-C₈ alkyl) or one to six carbon atoms (Cᵢ-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group may be optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Alkoxy” refers to a radical of the formula —ORₐ where Rₐ is an alkyl radical as defined above containing one to twelve carbon atoms. Unless stated otherwise specifically in the specification, an alkoxy group may be optionally substituted.

“Alkoxyalkyl” refers to a radical of the formula —R_{b}ORₐ where Rₐ is an alkyl radical as defined above containing one to twelve carbon atoms and R_{b} is an alkylene radical as defined above. Unless stated otherwise specifically in the specification, an alkoxyalkyl group may be optionally substituted.

“Alkylamino” refers to a radical of the formula —NHRₐ or —NRₐRₐ where each Rₐ is, independently, an alkyl radical as defined above containing one to twelve carbon atoms. Unless stated otherwise specifically in the specification, an alkylamino group may be optionally substituted.

“Alkylaminoalkyl” refers to a radical of the formula —R_{b}NHRₐ or —NRₐRₐ where each Rₐ is, independently, an alkyl radical as defined above containing one to twelve carbon atoms and R_{b} is an alkylene radical as defined above. Unless stated otherwise specifically in the specification, an alkylaminoalky group may be optionally substituted.

“Alkylsulfone” refers to a radical of the formula —S(O)₂Rₐ where Rₐ is an alkyl radical as defined above containing one to twelve carbon atoms and R_{b} is an alkylene radical as defined above. Unless stated otherwise specifically in the specification, an alkylsulfone group may be optionally substituted.

“Hydroxylalkyl” refers an alkyl radical as defined above containing one to twelve carbon atoms which has been substituted by one or more hydroxyl groups. Unless stated otherwise specifically in the specification, hydroxylalkyl group may be optionally substituted.

“Thioalkyl” refers to a radical of the formula —SRₐ where Rₐ is an alkyl radical as defined above containing one to twelve carbon atoms. Unless stated otherwise specifically in the specification, a thioalkyl group may be optionally substituted.

“Aryl” refers to a hydrocarbon ring system radical comprising hydrogen, 6 to 18 carbon atoms and at least one aromatic ring. For purposes of embodiments of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, αs-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. Unless stated otherwise specifically in the specification, an aralkyl group may be optionally substituted.

“Cycloalkyl” or “carbocyclic ring” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted.

“Cycloalkylalkyl” refers to a radical of the formula —R_{b}R_{d} where R_{b} is an alkylene chain as defined above and R_{d} is a cycloalkyl radical as defined above. Unless stated otherwise specifically in the specification, a cycloalkylalkyl group may be optionally substituted.

“Cycloalkoxyalkyl” refers to a radical of the formula —R_{b}ORₐ where Rₐ is a cycloalkyl radical as defined above and R_{b} is an alkyl radical as defined above containing one to twelve carbon atoms. Unless stated otherwise specifically in the specification, an alkoxyalkyl group may be optionally substituted.

“Fused” refers to any ring structure described herein which is fused to an existing ring structure in a compounds used in embodiments described herein. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group may be optionally substituted.

“Heterocyclylalkyl” refers to a radical of the formula —R_{b}Eₑ where R_{b} is an alkylene chain as defined above and Rₑ is a heterocyclyl radical as defined above, and if the heterocyclyl is a nitrogen-containing heterocyclyl, the heterocyclyl may be attached to the alkyl radical at the nitrogen atom. Unless stated otherwise specifically in the specification, a heterocyclylalkyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of the compounds used in certain embodiments of the invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group may be optionally substituted.

“Heteroarylalkyl” refers to a radical of the formula —R_{b}R_{f} where R_{b} is an alkylene chain as defined above and R_{f} is a heteroaryl radical as defined above. Unless stated otherwise specifically in the specification, a heteroarylalkyl group may be optionally substituted.

“Amino acid ester” refers to an amino acid having an ester group in place of the acid group. Unless stated otherwise specifically in the specification, an amino acid ester group may be optionally substituted.

The term “substituted” used herein means any of the above groups (i.e., alkyl, alkylene, alkoxy, alkoxyalkyl, alkylamino, alkylaminoalkyl, alkylsulfone, hydroxylalkyl, thioalkyl, aryl, aralkyl, cycloalkyl, cycloalkylalkyl, cycloalkoxyalkyl, haloalkyl, heterocyclyl, N-heterocyclyl, heterocyclylalkyl, heteroaryl, N-heteroaryl, heteroarylalkyl and/or amino acid ester) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —O₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, alkylamino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

“Prodrug” is meant to indicate a compound that may be converted under physiological conditions or by solvolysis to a biologically active compound that is administered in certain embodiments of the invention. Thus, the term “prodrug” refers to a pharmaceutically acceptable metabolic precursor of a compound administered in embodiments of the invention. In certain embodiments, a prodrug may be inactive when administered to a subject in need thereof, but is converted in vivo to an active compound. Prodrugs are typically rapidly transformed in vivo to yield an active compound, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release an active compound in vivo when such a prodrug is administered to a mammalian subject according to certain embodiments described herein. Prodrugs of a compound may be prepared by modifying functional groups present in the compound in such a way that the modifications are cleaved, either in routine manipulation or in vivo, to an active parent compound. Prodrugs include compounds wherein a hydroxy, amino or mercapto group is bonded to any group that, when the prodrug of the compound is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in compounds administered according to certain embodiments of the invention and the like.

Embodiments of the invention disclosed herein are also meant to encompass methods for administering, inter alia, all pharmaceutically acceptable compounds of the disclosed compounds, such as structures (I), (Ia), (Ib) and (Ic), being isotopically-labelled by having one or more atoms replaced by an atom having a different atomic mass or mass number. Examples of isotopes that can be incorporated into these compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the administration of the compounds, by characterizing, for example, the site or mode of action, or binding affinity to pharmacologically important site of action. Certain isotopically-labelled compounds of structure (I), (Ia), (Ib) or (Ic) for example, those incorporating a radioactive isotope, are useful in drug and/or substrate tissue distribution studies. The radioactive isotopes tritium, i.e., ³H, and carbon-^{14,} i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds (e.g., compounds of structure (I)) can generally be prepared by conventional techniques known to those skilled in the art, for example, replacing an appropriate isotopically-labeled reagent in place of the non-labeled reagent previously employed in a synthetic scheme.

“Stable compound” and “stable structure” are meant to indicate a compound that is sufficiently robust to survive isolation to a useful degree of purity from a reaction mixture, and formulation into an efficacious therapeutic agent.

“Patient” refers to a subject, such as a mammal, in need of medical care.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

A “pharmaceutical composition” refers to a formulation of a compound or compounds and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound or compounds which, when administered to a mammal, preferably a human, is sufficient to effect treatment, as defined below, of cancer in the mammal, preferably a human. The amount of a compound or compounds which constitutes a “therapeutically effective amount” will vary depending on the compound, combination of compounds, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, e.g., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

In certain embodiments of methods for administering compounds (including their pharmaceutically acceptable salts or tautomers) or compositions, the compounds may contain one or more stereocenters and may thus give rise to enantiomers, diastereomers, and other stereoisomeric forms that may be defined, in terms of absolute stereochemistry, as (R)- or (5)- or, as (D)- or (L)- for amino acids. The present methods are include compounds with all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When compounds described herein contain olefinic double bonds or other molecular features giving rise to geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The methods of the present invention include compounds having various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule, for example, the conversion of a ketone to an enol via a proton shift. Embodiments of the methods disclosed herein include tautomers of the disclosed compounds.

A “chemotherapeutic agent” or “anti-cancer agent” is a chemical which eradicates, stops or slows the growth of cancer cells.

A “reactive oxygen species” or “ROS” is a chemically reactive chemical containing oxygen, for example, peroxides, superoxide, hydroxyl radical, and singlet oxygen.

A “anthracycline” refers to a compound comprising the following core structure, which is optionally substituted at all available positions:

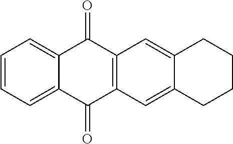 [SMILES: O=C1c2ccccc2C(=O)c2cc3c(cc21)CCCC3]

Exemplary anthracyclines include daunorubicin and idarubicin.

An “anthracenedione” refers to a class of compounds comprising the following core structure, which is optionally substituted at all available positions:

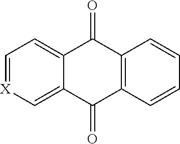

wherein X is C or N. Exemplary anthracenedione include mitoxantrone and pixantrone.

As noted above, in one embodiment, a method for treating cancer in a patient in need thereof is provided, the method comprising administering to the patient a therapeutically effective amount of the following therapeutic agents:

i) a PKM2 activator; and

ii) an anti-cancer drug having a mechanism of action that increases production of reactive oxygen species in cancer cells upon administration to the patient.

In certain embodiments, the PKM2 activator and/or anti-cancer drug are provided in the form of a stereoisomer, pharmaceutically acceptable salt, tautomer or prodrug of the PKM2 activator and/or anti-cancer drug.

Exemplary PKM2 activators and anti-cancer drugs having a mechanism of action that increases production of ROS in cancer cells are described herein below.

### I. PKM2 Compounds

In some embodiments, the PKM2 activator is a PKM2 activator as disclosed in U.S. Pat. No. 9,394,257, the full disclosure of which is incorporated herein by reference in its entirety. Accordingly, in one embodiment of the disclosed methods the PKM2 activator has the following structure (I):

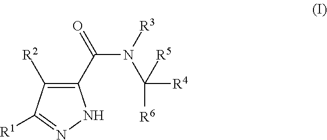

or a stereoisomer, pharmaceutically acceptable salt, tautomer or prodrug thereof,

wherein:

R¹ is cycloalkyl, haloalkyl, halo, nitrile or amino;

R² is H or halo;

R³ is alkyl, alkoxyalkyl, cycloalkoxyalkyl, cycloalkyl, cycloalkylalkyl, heterocyclyl, heterocyclylalkyl, heteroaryl, heteroarylalkyl or aralkyl

R⁴ is aryl or heteroaryl

R⁵ and R⁶ are each independently H or alkyl.

In some embodiments, R⁴ is aryl. In other embodiments, R⁴ is heteroaryl.

In some more specific embodiments, R⁴ has one of the following structures (A), (B) or (C):

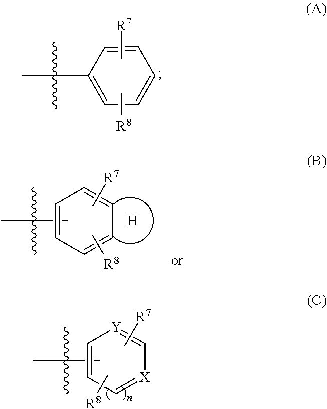

wherein:

H represents a 5 or 6-membered heterocyclic ring;

X is O, N, N⁺—O⁻ or S;

Y is CH or N;

R⁷ and R⁸ are each independently H, alkyl, alkoxy, halo, hydroxyl, hydroxylalkyl, amino, aminoalkyl, alkylaminoalkyl, nitrile, nitro, —O(CH₂)ₘP(═O)(OH)₂, amino acid ester; and

m and n are each independently 0 or 1, wherein all valences are satisfied.

In some embodiments of the foregoing, R⁴ has structure (A). For example, in some embodiments, R⁷ and R⁸ are each independently H, halo or amino.

In other embodiments, R⁴ has the following structure:

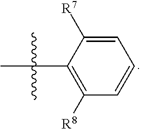

For example, in some embodiments R⁷ is H or amino, and in other embodiments R⁸ is chloro or fluoro.

In some other more specific embodiments, R⁴ has one of the following structures:

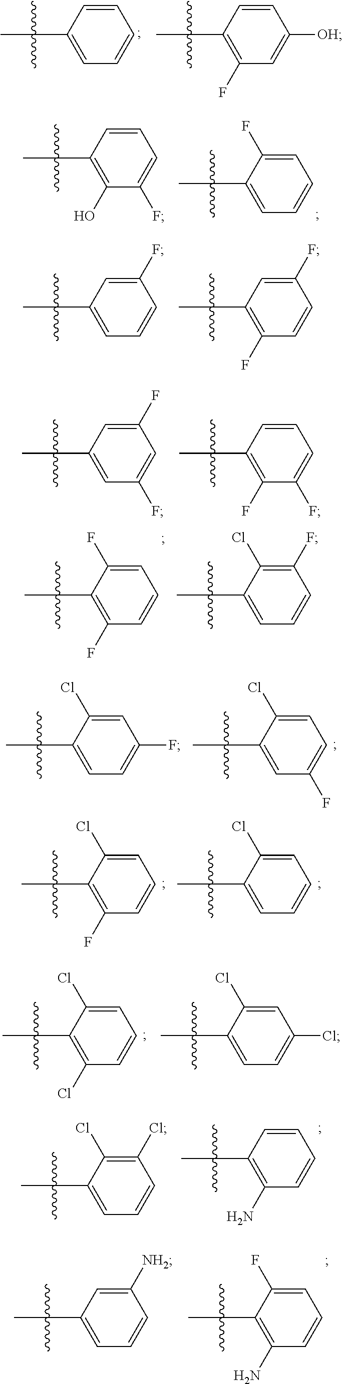

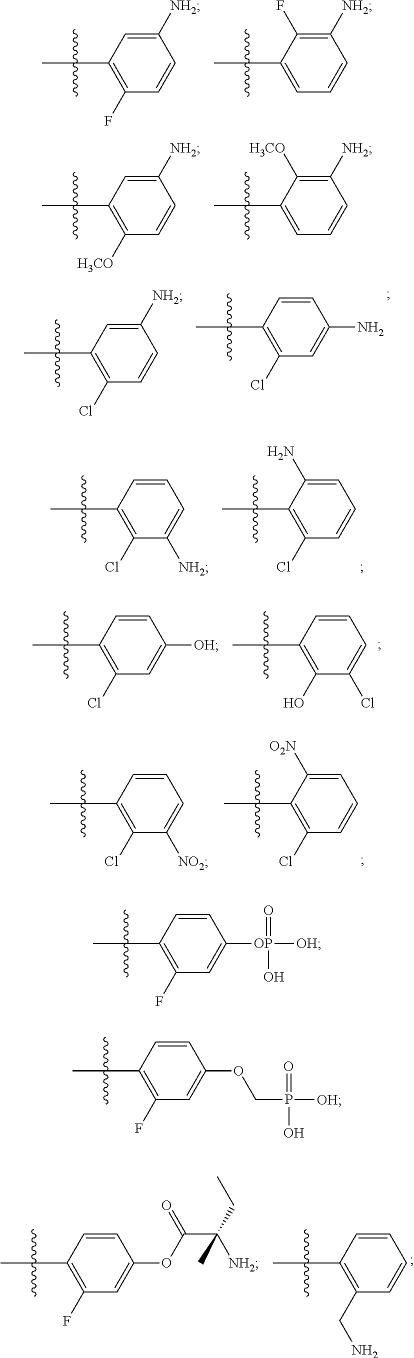

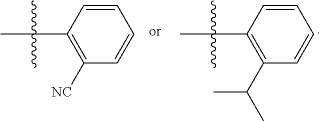

In other embodiments, R⁴ has structure (B). For example, R⁴ has one of the following structures in certain embodiments:

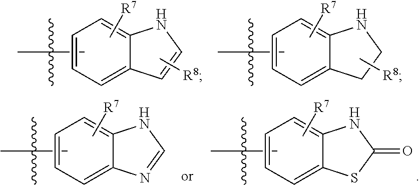

In some of the above embodiments, R⁷ and R⁸ are each H, and in other embodiments R⁷ or R⁸ is halo or alkylaminoalkyl.

In still other embodiments, R⁴ has one of the following structures:

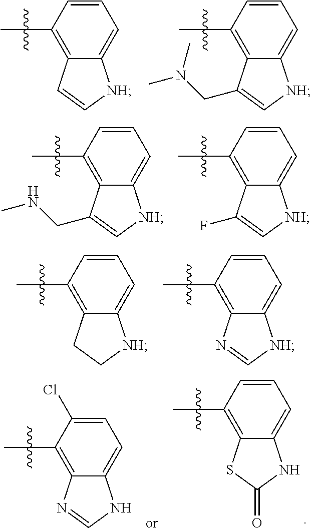

In yet other exemplary embodiments, R⁴ has structure (C). For example, in some embodiments R⁴ has one of the following structures:

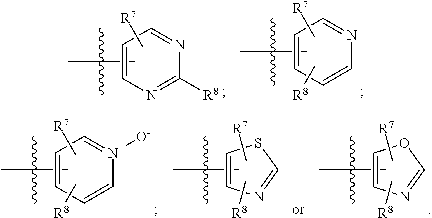

In some embodiments of the foregoing, R⁷ and R⁸ are each H. In other embodiments, R⁷ or R⁸ is halo, amino or hydroxylalkyl.

In some other specific examples, R⁴ has one of the following structures:

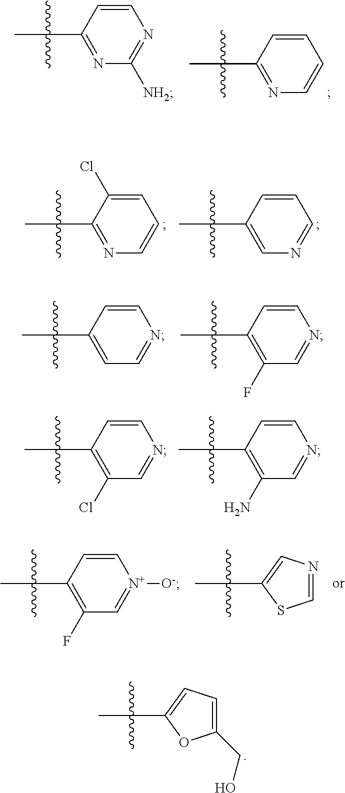

In still other embodiments, R³ has one of the following structures (D), (E) or (F):

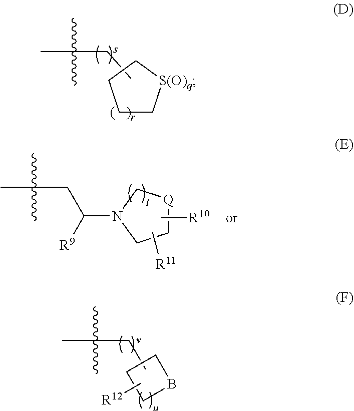

wherein:

Q is CH₂, O, NR¹³, CF₂, or S(O)_{w};

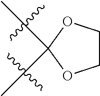 [SMILES: *C1(*)OCCO1]

B is CH₂, O, NR¹⁴, C(═O) or

R⁹, R¹¹ and R¹³ are each independently H or alkyl;

R¹⁰ is H, hydroxyl, halo, alkoxy or alkyl;

R¹² is H, amino or alkoxy;

R¹⁴ is H, alkyl or alkyl sulfone;

q, v and w are each independently 0, 1 or 2;

r and s are each independently 1 or 2;

t is 1, 2 or 3; and

u is 0, 1, 2 or 3.

In certain embodiments, R³ has structure (D).

In some embodiments, s is 1. In other embodiments, s is 2. In still other embodiments, r is 1. In more other embodiments, r is 2. In some more embodiments, q is 0. In yet other embodiments, q is 1. In other embodiments, q is 2.

In some more specific examples, R³ has one of the following structures:

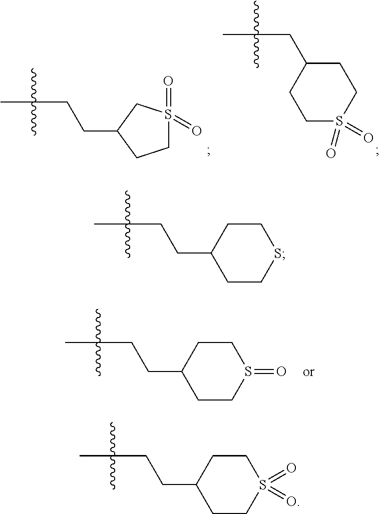 [SMILES: *CCC1CCS(=O)(=O)C1 ; *CC1CCS(=O)(=O)CC1 ; *CCC1CCSCC1 ; *CCC1CCS(=O)CC1 ; *CCC1CCS(=O)(=O)CC1]

In some other embodiments, R³ has structure (E).

In some embodiments, Q is CH₂. In other embodiments, Q is SO₂. In more embodiments, Q is O. In yet other embodiments, Q is CHF₂. In still other embodiments, Q is NR¹³.

In some of the foregoing embodiments, R¹³ is methyl or ethyl.

In other of the foregoing embodiments, R¹⁰ and R¹¹ are each H. In yet other embodiments, R¹²⁰ is methyl, fluoro, hydroxyl or methoxy.

In still other specific embodiments, R³ has one of the following structures:

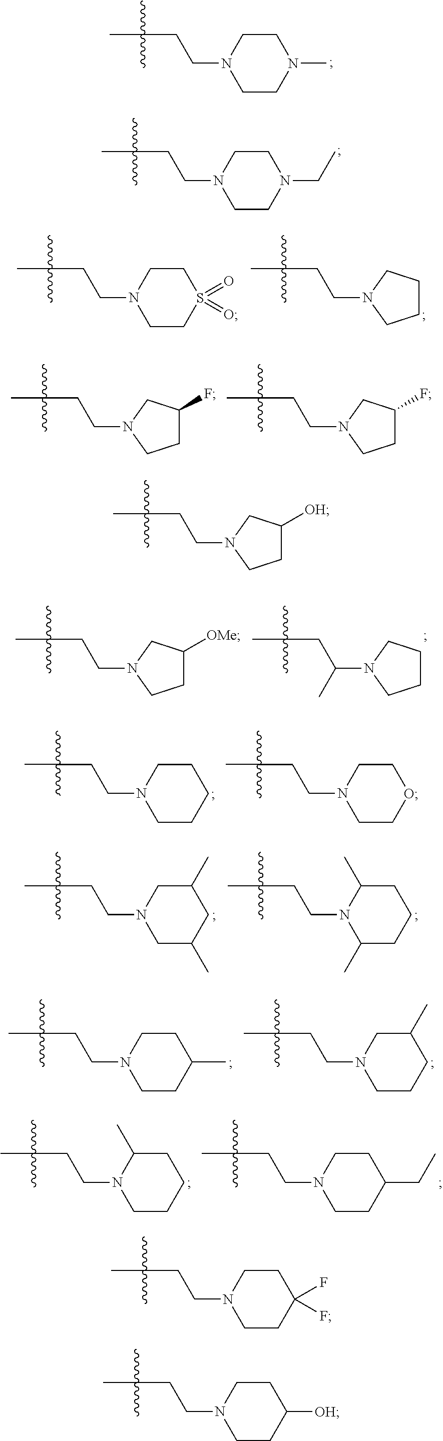

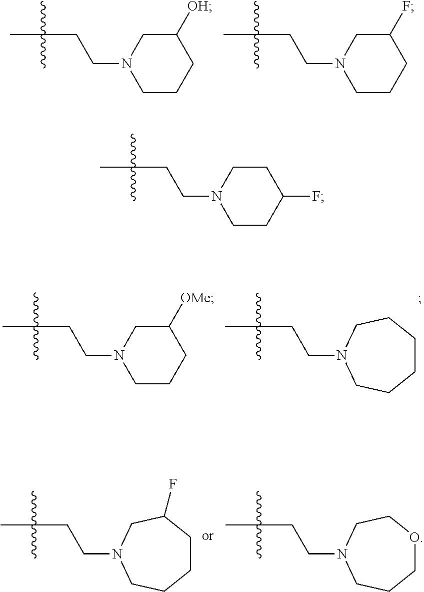

In still other specific embodiments, R³ has structure (F). In some of these embodiments, B is CH₂. In other embodiments, R¹² is H. In some embodiments, R¹² is alkoxy. In still other embodiments, R¹² is methoxy, ethoxy or isopropoxy.

In some other exemplary embodiments, R³ has one of the following structures:

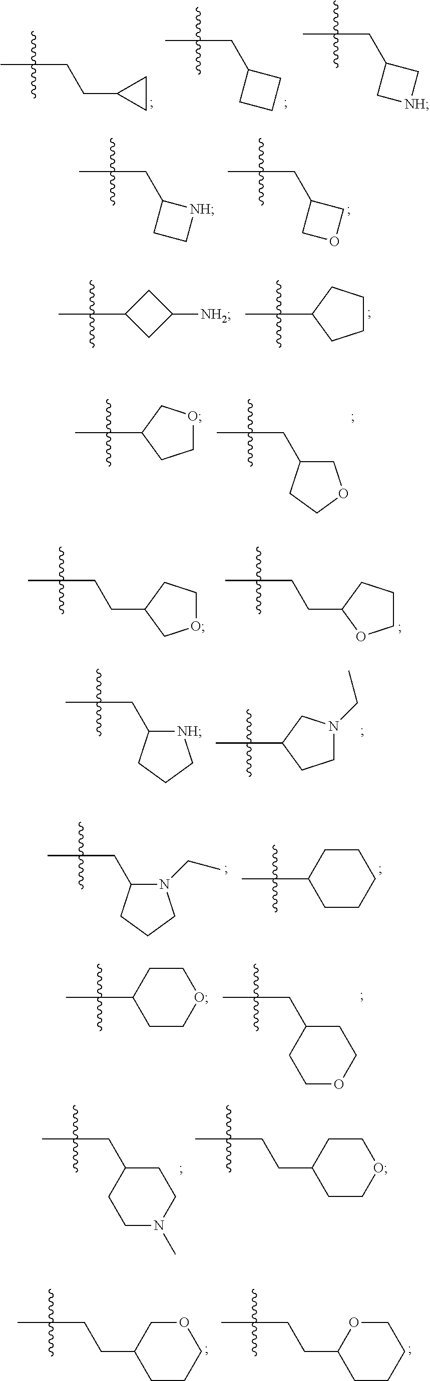

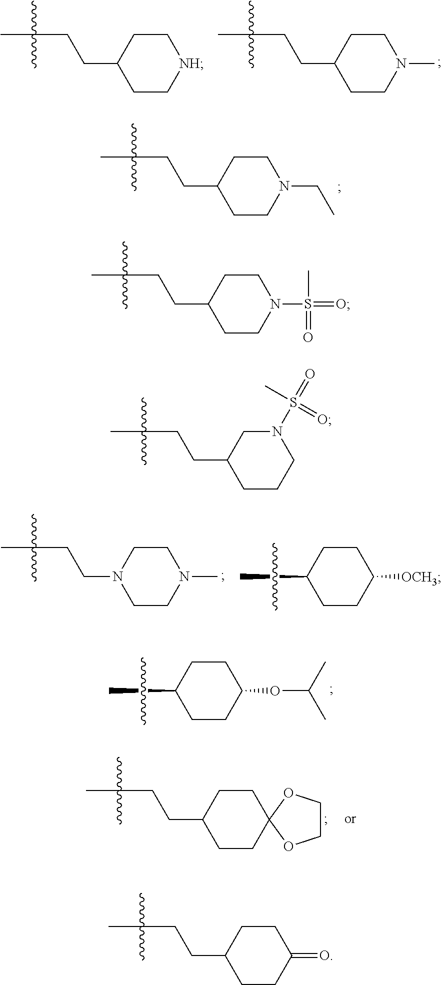

In still other embodiments, R³ is alkoxyalkyl, and in other embodiments R³ is alkyl, for example, in some embodiments the alkyl is substituted with one or more substituents selected from hydroxyl, halo, amino, alkylamino, alkoxy and alkylsulfone. In other embodiments, R³ is heteroaryl. In yet other embodiments, R³ is cycloalkoxyalkyl. In more embodiments, R³ is aralkyl.

In other embodiments, R⁵ and R⁶ are each H.

In some of any of the preceding embodiments, R² is H, and in other embodiments R² is F.

In other embodiments of any of the foregoing embodiments, R¹ is CF₃. In other embodiments, R¹ is Cl. In still other examples, R¹ is Br. In some embodiments, R¹ is cyclopropyl. In other embodiments, R¹ is nitrile. In yet other embodiments, R¹ is amino.

In some embodiments the PKM2 activator has the following structure (Ia′):

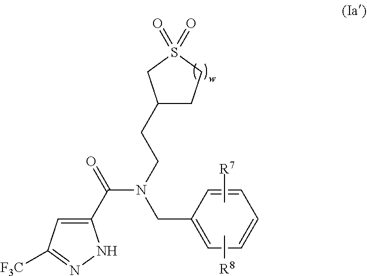

or a stereoisomer, pharmaceutically acceptable salt, tautomer or prodrug thereof,

wherein:

R⁷ and R⁸ are each independently H, alkyl, alkoxy, halo, hydroxyl, hydroxylalkyl, amino, aminoalkyl, alkylaminoalkyl, nitrile, nitro, —O(CH₂)ₘP(═O)(OH)₂, amino acid ester; and

w is 1 or 2.

For example, in some embodiments the PKM2 activator has the following structure (Ia):

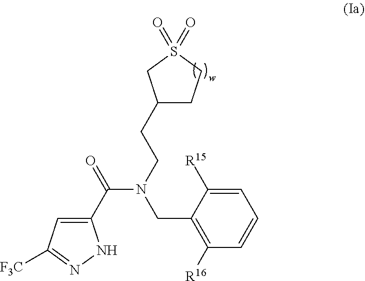

or a stereoisomer, pharmaceutically acceptable salt, tautomer or prodrug thereof,

wherein:

R¹⁵ is halo;

R¹⁶ is H or NH₂; and

w is 1 or 2.

In some embodiments of structure (Ia), R¹⁵ is chloro. In other embodiments, R¹⁵ is fluoro.

In some other embodiments of structure (Ia), R¹⁶ is H. In other embodiments, R¹⁶ is NH₂.

In still other embodiments of structure (Ia), w is 1. In other embodiments, w is 2.

In other specific embodiments the PKM2 activator has the following structure (Ib′):

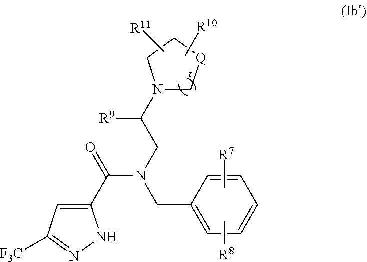

or a stereoisomer, pharmaceutically acceptable salt, tautomer or prodrug thereof,

wherein:

Q is CH₂, O, NR¹³, CF₂, or S(O)_{w};

R⁷ and R⁸ are each independently H, alkyl, alkoxy, halo, hydroxyl, hydroxylalkyl, amino, aminoalkyl, alkylaminoalkyl, nitrile, nitro, —O(CH₂)ₘP(═O)(OH)₂, amino acid ester;

R⁹, R¹¹ and R¹³ are each independently H or alkyl;

R¹⁰ is H, hydroxyl, halo, alkoxy or alkyl;

w is 0, 1 or 2; and

t is 1, 2 or 3.

For example, in other specific embodiments the PKM2 activator has the following structure (Ib):

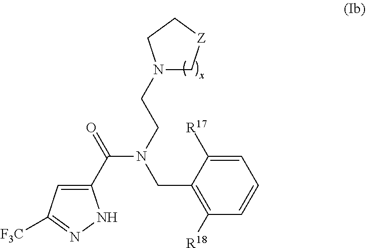

or a stereoisomer, pharmaceutically acceptable salt, tautomer or prodrug thereof,

wherein:

R¹⁷ is halo;

R¹⁸ is H or NH₂;

Z is CH₂, O, NH, NR¹⁹, CHR²⁰ or CF₂;

R¹⁹ is alkyl;

R²⁰ is alkoxy, hydroxyl or halo; and

x is 0, 1, 2 or 3.

For example, in some embodiments, R¹⁷ is chloro. In other embodiments, R¹⁸ is NH₂.

In still other embodiments of structure (Ib), Z is CHOH. In other embodiments, Z is CHOCH₃. In more embodiments, Z is CHF, and in other embodiments Z is O.

In more embodiments, x is 1, and in other embodiments x is 2.

In other exemplary embodiments the PKM2 activator has the following structure (Ic′):

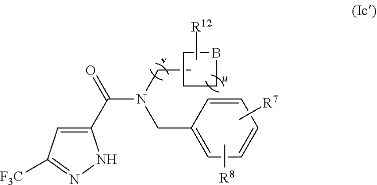

or a stereoisomer, pharmaceutically acceptable salt, tautomer or prodrug thereof,

wherein:

B is CH₂, O, NR¹⁴, C(═O) or

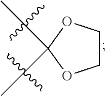 [SMILES: *C1(*)OCCO1]

R⁷ and R⁸ are each independently H, alkyl, alkoxy, halo, hydroxyl, hydroxylalkyl, amino, aminoalkyl, alkylaminoalkyl, nitrile, nitro, —O(CH₂)ₘP(═O)(OH)₂, amino acid ester;

R¹² is H, amino or alkoxy;

v is 0, 1 or 2; and

u is0, 1,2or3.

For example, in other exemplary embodiments the PKM2 activator has the following structure (Ic):

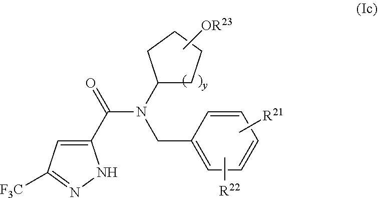

or a stereoisomer, pharmaceutically acceptable salt, tautomer or prodrug thereof,

wherein:

R²¹ and R²² are each independently H or halo;

R²³ is H or alkyl; and

y is 1 or 2.

In some embodiments, R²¹ is chloro. In other embodiments, R^{21 is F.}

In still other embodiments, R²² is H. In other embodiments, R²³ is methyl, ethyl or isopropyl.

In certain exemplary embodiments, y is 1. In other embodiments, y is 2.

In some other embodiments of the disclosed methods, the PKM2 activator has the following structure (II):

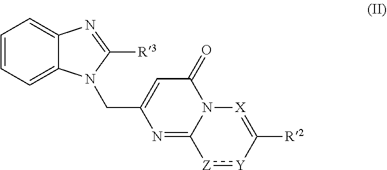

or a stereoisomer, pharmaceutically acceptable salt, tautomer or prodrug thereof,

wherein:

X is N or CR′¹;

Y is S or CR′¹;

Z is a direct bond or CR′¹;

R′¹ and R′² are, at each occurrence, independently, H, C₁-C₆ alkyl, C₁-C₆ alkoxy, halo or aryl;

R′³ is H, C₁-C₆ alkyl or aralkyl; and

[symbol] represents a single or double bond.

In some embodiments, X is N, Y is S, Z is a direct bond and [symbol] represents a single bond. In some of those embodiments, R′² is ethyl and R′³ is H.

In other embodiments, X is CR′¹, Y is S, Z is a direct bond and [symbol] represents a single bond. In some specific embodiments, R′¹ is H, methyl, or aryl, e.g., phenyl. In more specific embodiments, R′² is H or methyl and R′³ is H.

In some embodiments, X is CR′¹, Y is CR′¹, Z is CR′¹ and [symbol] represents a double bond. In some of those embodiments, R′¹ is H, methyl or ethyl. In certain related embodiments, R′² is H, methyl, methoxy or halo, e.g., chloro. In some embodiments, R′³ is H, methyl or aralkyl, e.g., benzyl.

In some embodiments of the disclosed methods, the PKM2 activator has the following structure (III):

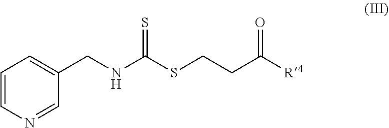

or a stereoisomer, pharmaceutically acceptable salt, tautomer or prodrug thereof,

wherein:

R′⁴ is a substituted or unsubstituted monocyclic aryl, bicyclic aryl, monocyclic heteroaryl, bicyclic heteroaryl or aralkenyl.

In some of the foregoing embodiments, R′⁴ is an unsubstituted phenyl. In other embodiments, R′⁴ is a phenyl substituted with 1, 2 or 3 substituents selected from the group consisting of phenyl is substituted with halo (e.g., chloro, fluoro), cyano, hydroxyl, nitro, alkylamino (e.g., —N(CH₃)₂), aralkoxy (e.g., benzyloxy), alkoxy (e.g., methoxy) and haloalkyl (e.g., trifluoromethyl).

In some embodiments, R′⁴ is a bicyclic aryl (e.g., naphthalenyl).

In some embodiments, R′⁴ is a monocyclic heteroaryl (e.g., furanyl, thiophenyl, pyrrolyl, thiazolyl or pyridinyl).

In some specific embodiments, R′⁴ is a bicyclic heteroaryl (e.g., indolyl, azaindolyl, imidazo[1,2-a]pyridinyl).

In some specific embodiments, R′⁴ is an aralkenyl. In more specific embodiments R′⁴ is

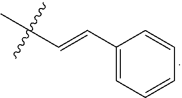 [SMILES: */C=C/c1ccccc1]

In certain embodiments of the disclosed methods, the PKM2 activator has the following structure (IV):

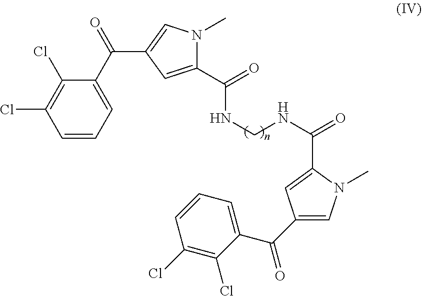

or a stereoisomer, pharmaceutically acceptable salt, tautomer or prodrug thereof,

wherein:

n is greater than 0.

In certain embodiments of the foregoing, n is 1. In certain other embodiments of the foregoing, n is 2.

In other certain embodiments, the PKM2 activator is selected from Table 1, or a stereoisomer, pharmaceutically acceptable salt, tautomer or prodrug of a compound in Table 1.

TABLE 1
Exemplary PKM2 Activators

| | | PKM₂ | |
|---|---|---|---|
| | | AC₅₀ (nM)/ | |
| | | Max. Resp.* | |
| Compound | Structure | (%) | Note^{†} |
| 1 | 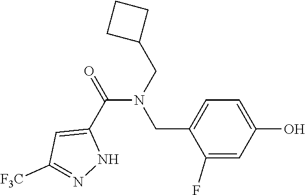 [SMILES: O=C(c1cc(C(F)(F)F)n[nH]1)N(Cc1ccc(O)cc1F)CC1CCC1] | 11/100 | — |
| 2 | 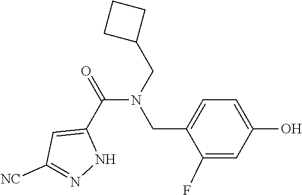 [SMILES: N#Cc1cc(C(=O)N(Cc2ccc(O)cc2F)CC2CCC2)[nH]n1] | 3788/39 | — |
| 3 | 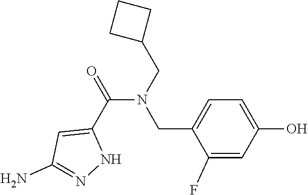 [SMILES: Nc1cc(C(=O)N(Cc2ccc(O)cc2F)CC2CCC2)[nH]n1] | 43850/45 | — |
| 4 | 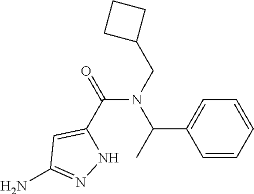 [SMILES: CC(c1ccccc1)N(CC1CCC1)C(=O)c1cc(N)n[nH]1] | 1482/52 | — |
| 5 | 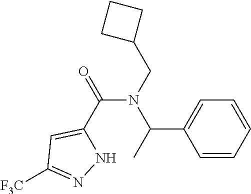 [SMILES: CC(c1ccccc1)N(CC1CCC1)C(=O)c1cc(C(F)(F)F)n[nH]1] | 4651/106 | — |
| 6 | 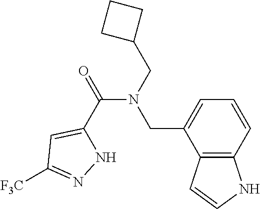 [SMILES: O=C(c1cc(C(F)(F)F)n[nH]1)N(Cc1cccc2[nH]ccc12)CC1CCC1] | 213/100 | — |
| 7 | 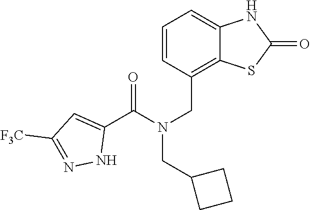 [SMILES: O=C(c1cc(C(F)(F)F)n[nH]1)N(Cc1cccc2[nH]c(=O)sc12)CC1CCC1] | 3446/87 | — |
| 8 | 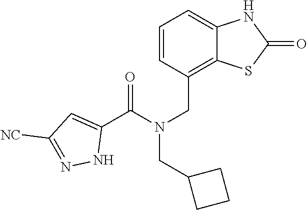 [SMILES: N#Cc1cc(C(=O)N(Cc2cccc3[nH]c(=O)sc23)CC2CCC2)[nH]n1] | NA | — |
| 9 | 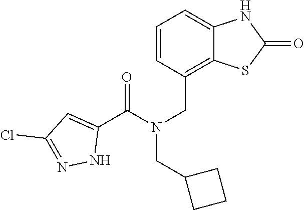 [SMILES: O=C(c1cc(Cl)n[nH]1)N(Cc1cccc2[nH]c(=O)sc12)CC1CCC1] | 15400/80 | — |
| 10 | 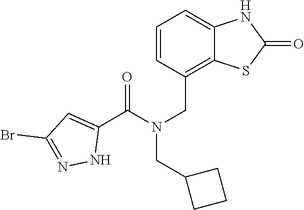 [SMILES: O=C(c1cc(Br)n[nH]1)N(Cc1cccc2[nH]c(=O)sc12)CC1CCC1] | 2740/127 | — |
| 11 | 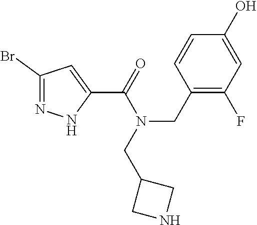 [SMILES: O=C(c1cc(Br)n[nH]1)N(Cc1ccc(O)cc1F)CC1CNC1] | 10400/61 | — |
| 12 | 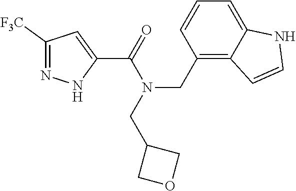 [SMILES: O=C(c1cc(C(F)(F)F)n[nH]1)N(Cc1cccc2[nH]ccc12)CC1COC1] | 3271/101 | — |
| 13 | 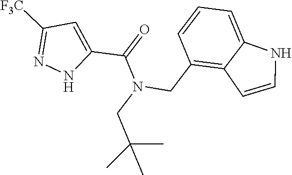 [SMILES: CC(C)(C)CN(Cc1cccc2[nH]ccc12)C(=O)c1cc(C(F)(F)F)n[nH]1] | 49110/33 | — |
| 14 | 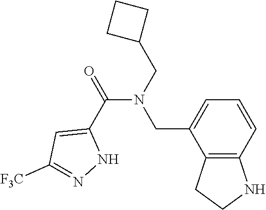 [SMILES: O=C(c1cc(C(F)(F)F)n[nH]1)N(Cc1cccc2c1CCN2)CC1CCC1] | 1700/81 | — |
| 15 | 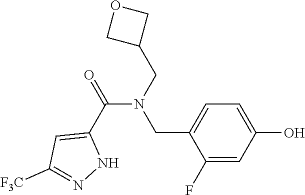 [SMILES: O=C(c1cc(C(F)(F)F)n[nH]1)N(Cc1ccc(O)cc1F)CC1COC1] | 150/102 | — |
| 16 | 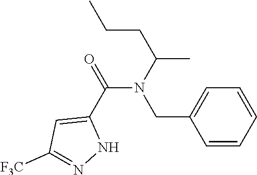 [SMILES: CCCC(C)N(Cc1ccccc1)C(=O)c1cc(C(F)(F)F)n[nH]1] | 74820/22 | — |
| 17 | 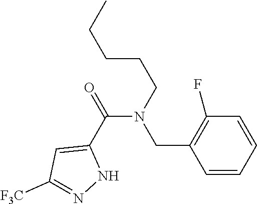 [SMILES: CCCCCN(Cc1ccccc1F)C(=O)c1cc(C(F)(F)F)n[nH]1] | 138/83 | — |
| 18 | 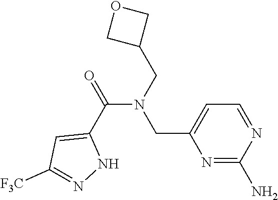 [SMILES: Nc1nccc(CN(CC2COC2)C(=O)c2cc(C(F)(F)F)n[nH]2)n1] | NA | — |
| 19 | 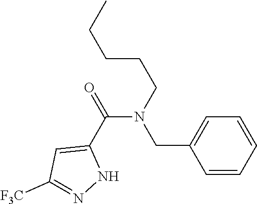 [SMILES: CCCCCN(Cc1ccccc1)C(=O)c1cc(C(F)(F)F)n[nH]1] | 920/76 | — |
| 20 | 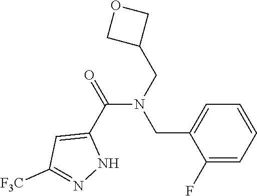 [SMILES: O=C(c1cc(C(F)(F)F)n[nH]1)N(Cc1ccccc1F)CC1COC1] | 12000/70 | — |
| 21 | 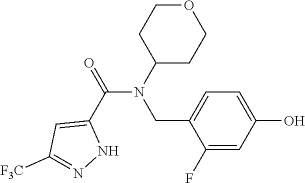 [SMILES: O=C(c1cc(C(F)(F)F)n[nH]1)N(Cc1ccc(O)cc1F)C1CCOCC1] | >10000 | — |
| 22 | 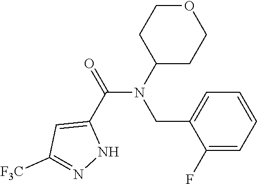 [SMILES: O=C(c1cc(C(F)(F)F)n[nH]1)N(Cc1ccccc1F)C1CCOCC1] | 1516/99 | — |
| 23 | 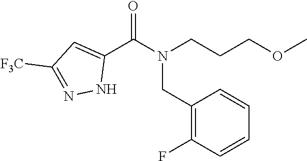 [SMILES: COCCCN(Cc1ccccc1F)C(=O)c1cc(C(F)(F)F)n[nH]1] | 7540/75 | — |
| 24 | 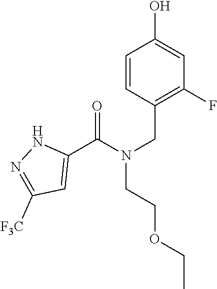 [SMILES: CCOCCN(Cc1ccc(O)cc1F)C(=O)c1cc(C(F)(F)F)n[nH]1] | 210/100 | — |
| 25 | 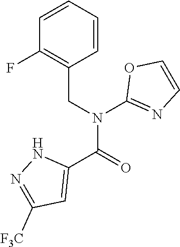 [SMILES: O=C(c1cc(C(F)(F)F)n[nH]1)N(Cc1ccccc1F)c1ncco1] | 53000/35 | — |
| 26 | 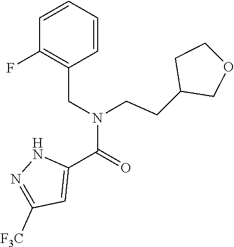 [SMILES: O=C(c1cc(C(F)(F)F)n[nH]1)N(CCC1CCOC1)Cc1ccccc1F] | 377/99 | — |
| 27 | 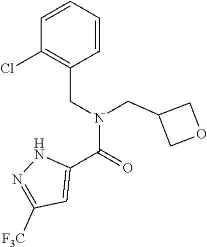 [SMILES: O=C(c1cc(C(F)(F)F)n[nH]1)N(Cc1ccccc1Cl)CC1COC1] | 33000/47 | — |
| 28 | 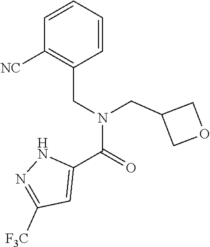 [SMILES: N#Cc1ccccc1CN(CC1COC1)C(=O)c1cc(C(F)(F)F)n[nH]1] | NA | — |
| 29 | 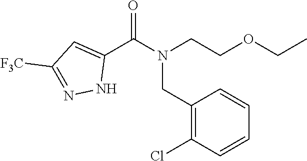 [SMILES: CCOCCN(Cc1ccccc1Cl)C(=O)c1cc(C(F)(F)F)n[nH]1] | 4640/87 | — |
| 30 | 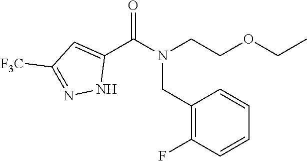 [SMILES: CCOCCN(Cc1ccccc1F)C(=O)c1cc(C(F)(F)F)n[nH]1] | 9770/72 | — |
| 31 | 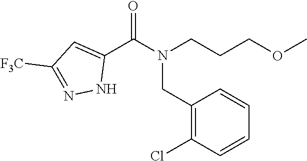 [SMILES: COCCCN(Cc1ccccc1Cl)C(=O)c1cc(C(F)(F)F)n[nH]1] | 9590/82 | — |
| 32 | 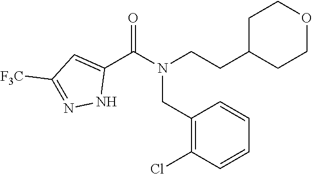 [SMILES: O=C(c1cc(C(F)(F)F)n[nH]1)N(CCC1CCOCC1)Cc1ccccc1Cl] | 1002/107 | — |
| 33 | 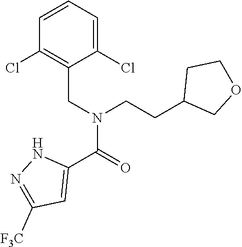 [SMILES: O=C(c1cc(C(F)(F)F)n[nH]1)N(CCC1CCOC1)Cc1c(Cl)cccc1Cl] | 4026/81 | — |
| 34 | 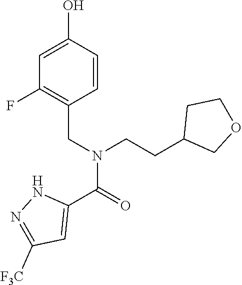 [SMILES: O=C(c1cc(C(F)(F)F)n[nH]1)N(CCC1CCOC1)Cc1ccc(O)cc1F] | 77/109 | — |
| 35 | 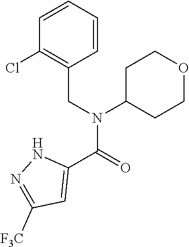 [SMILES: O=C(c1cc(C(F)(F)F)n[nH]1)N(Cc1ccccc1Cl)C1CCOCC1] | 5080/59 | — |
| 36 | 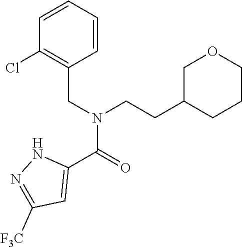 [SMILES: O=C(c1cc(C(F)(F)F)n[nH]1)N(CCC1CCCOC1)Cc1ccccc1Cl] | 1218/88 | — |
| 37 | 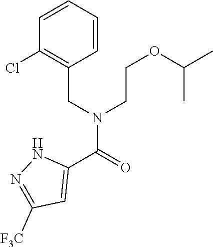 [SMILES: CC(C)OCCN(Cc1ccccc1Cl)C(=O)c1cc(C(F)(F)F)n[nH]1] | 42000/37 | — |
| 38 | 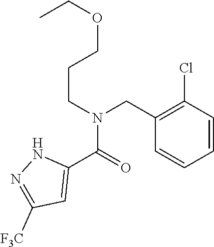 [SMILES: CCOCCCN(Cc1ccccc1Cl)C(=O)c1cc(C(F)(F)F)n[nH]1] | 33000/47 | — |
| 39 | 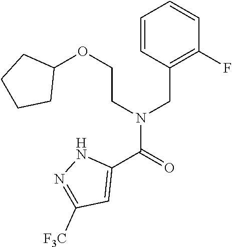 [SMILES: O=C(c1cc(C(F)(F)F)n[nH]1)N(CCOC1CCCC1)Cc1ccccc1F] | 47000/30 | — |
| 40 | 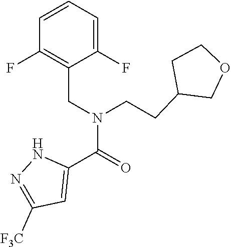 [SMILES: O=C(c1cc(C(F)(F)F)n[nH]1)N(CCC1CCOC1)Cc1c(F)cccc1F] | 1171/114 | — |
| 41 | 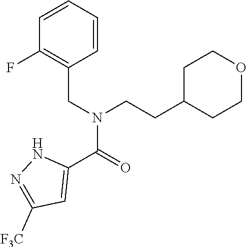 [SMILES: O=C(c1cc(C(F)(F)F)n[nH]1)N(CCC1CCOCC1)Cc1ccccc1F] | 1258/118 | — |
| 42 | 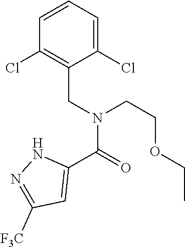 [SMILES: CCOCCN(Cc1c(Cl)cccc1Cl)C(=O)c1cc(C(F)(F)F)n[nH]1] | 8780/76 | — |
| 43 | 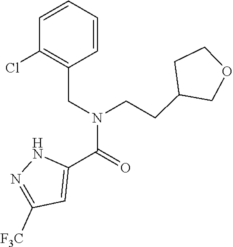 [SMILES: O=C(c1cc(C(F)(F)F)n[nH]1)N(CCC1CCOC1)Cc1ccccc1Cl] | 1330/110 | — |
| 44 | 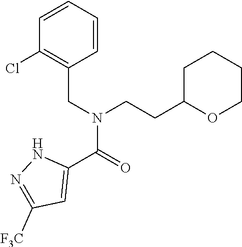 [SMILES: O=C(c1cc(C(F)(F)F)n[nH]1)N(CCC1CCCCO1)Cc1ccccc1Cl] | 50000/32 | — |
| 45 | 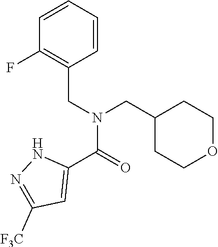 [SMILES: O=C(c1cc(C(F)(F)F)n[nH]1)N(Cc1ccccc1F)CC1CCOCC1] | 25150/52 | — |
| 46 | 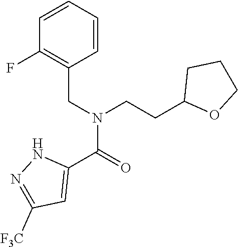 [SMILES: O=C(c1cc(C(F)(F)F)n[nH]1)N(CCC1CCCO1)Cc1ccccc1F] | 13410/70 | — |
| 47 | 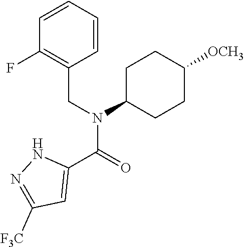 [SMILES: CO[C@H]1CC[C@H](N(Cc2ccccc2F)C(=O)c2cc(C(F)(F)F)n[nH]2)CC1] | 435/89 | — |
| 48 | 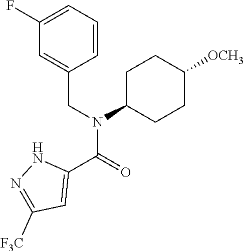 [SMILES: CO[C@H]1CC[C@H](N(Cc2cccc(F)c2)C(=O)c2cc(C(F)(F)F)n[nH]2)CC1] | 8580/80 | — |
| 49 | 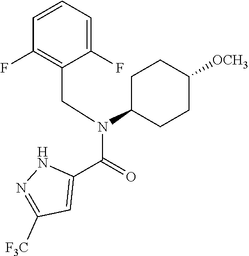 [SMILES: CO[C@H]1CC[C@H](N(Cc2c(F)cccc2F)C(=O)c2cc(C(F)(F)F)n[nH]2)CC1] | 914/122 | — |
| 50 | 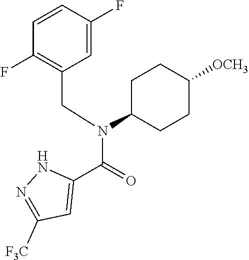 [SMILES: CO[C@H]1CC[C@H](N(Cc2cc(F)ccc2F)C(=O)c2cc(C(F)(F)F)n[nH]2)CC1] | 1667/90 | — |
| 51 | 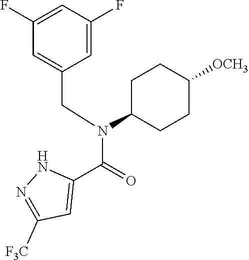 [SMILES: CO[C@H]1CC[C@H](N(Cc2cc(F)cc(F)c2)C(=O)c2cc(C(F)(F)F)n[nH]2)CC1] | 50380/31 | — |
| 52 | 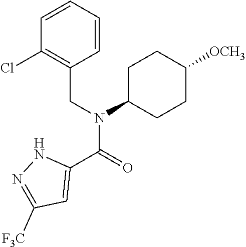 [SMILES: CO[C@H]1CC[C@H](N(Cc2ccccc2Cl)C(=O)c2cc(C(F)(F)F)n[nH]2)CC1] | 879/100 | — |
| 53 | 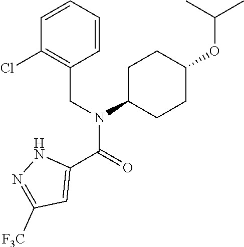 [SMILES: CC(C)O[C@H]1CC[C@H](N(Cc2ccccc2Cl)C(=O)c2cc(C(F)(F)F)n[nH]2)CC1] | 2287/63 | — |
| 54 | 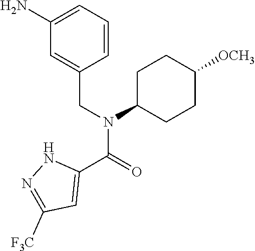 [SMILES: CO[C@H]1CC[C@H](N(Cc2cccc(N)c2)C(=O)c2cc(C(F)(F)F)n[nH]2)CC1] | 365/93 | TFA |
| 55 | 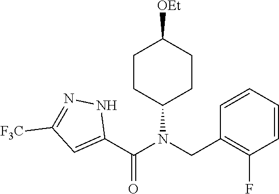 [SMILES: CCO[C@H]1CC[C@H](N(Cc2ccccc2F)C(=O)c2cc(C(F)(F)F)n[nH]2)CC1] | 469/101 | — |
| 56 | 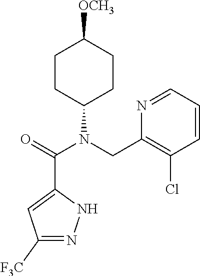 [SMILES: CO[C@H]1CC[C@H](N(Cc2ncccc2Cl)C(=O)c2cc(C(F)(F)F)n[nH]2)CC1] | 2580/48 | TFA |
| 57 | 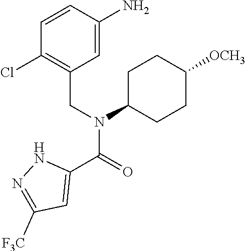 [SMILES: CO[C@H]1CC[C@H](N(Cc2cc(N)ccc2Cl)C(=O)c2cc(C(F)(F)F)n[nH]2)CC1] | TBD | TFA |
| 58 | 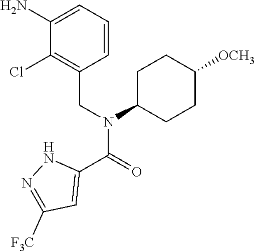 [SMILES: CO[C@H]1CC[C@H](N(Cc2cccc(N)c2Cl)C(=O)c2cc(C(F)(F)F)n[nH]2)CC1] | TBD | TFA |
| 59 | 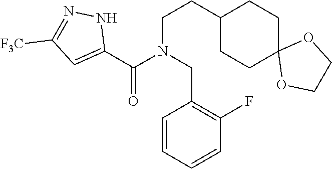 [SMILES: O=C(c1cc(C(F)(F)F)n[nH]1)N(CCC1CCC2(CC1)OCCO2)Cc1ccccc1F] | 494/100 | — |
| 60 | 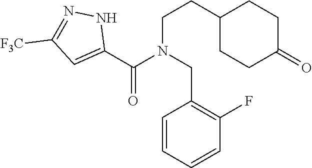 [SMILES: O=C1CCC(CCN(Cc2ccccc2F)C(=O)c2cc(C(F)(F)F)n[nH]2)CC1] | 101/105 | — |
| 61 | 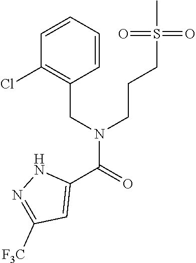 [SMILES: CS(=O)(=O)CCCN(Cc1ccccc1Cl)C(=O)c1cc(C(F)(F)F)n[nH]1] | NA | — |
| 62 | 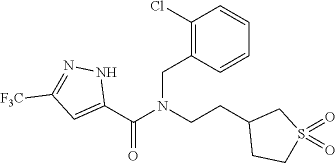 [SMILES: O=C(c1cc(C(F)(F)F)n[nH]1)N(CCC1CCS(=O)(=O)C1)Cc1ccccc1Cl] | 273/105 | — |
| 63 | 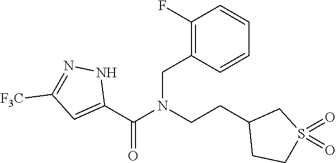 [SMILES: O=C(c1cc(C(F)(F)F)n[nH]1)N(CCC1CCS(=O)(=O)C1)Cc1ccccc1F] | 240/87 | — |
| 64 | 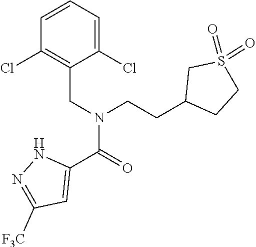 [SMILES: O=C(c1cc(C(F)(F)F)n[nH]1)N(CCC1CCS(=O)(=O)C1)Cc1c(Cl)cccc1Cl] | 3780/71 | — |
| 65 | 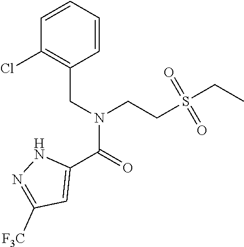 [SMILES: CCS(=O)(=O)CCN(Cc1ccccc1Cl)C(=O)c1cc(C(F)(F)F)n[nH]1] | NA | — |
| 66 | 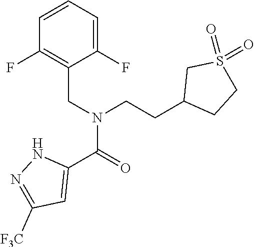 [SMILES: O=C(c1cc(C(F)(F)F)n[nH]1)N(CCC1CCS(=O)(=O)C1)Cc1c(F)cccc1F] | 375/102 | — |
| 67 | 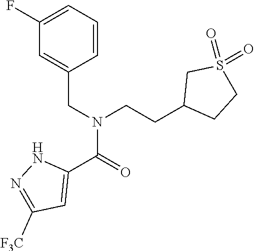 [SMILES: O=C(c1cc(C(F)(F)F)n[nH]1)N(CCC1CCS(=O)(=O)C1)Cc1cccc(F)c1] | 1626/95 | — |
| 68 | 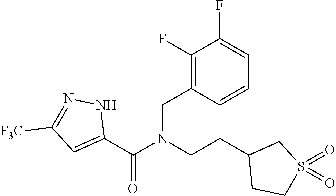 [SMILES: O=C(c1cc(C(F)(F)F)n[nH]1)N(CCC1CCS(=O)(=O)C1)Cc1cccc(F)c1F] | 766/103 | — |
| 69 | 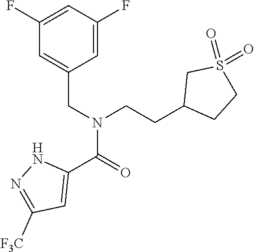 [SMILES: O=C(c1cc(C(F)(F)F)n[nH]1)N(CCC1CCS(=O)(=O)C1)Cc1cc(F)cc(F)c1] | 1835/74 | — |
| 70 | 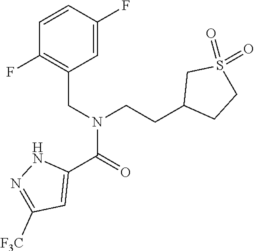 [SMILES: O=C(c1cc(C(F)(F)F)n[nH]1)N(CCC1CCS(=O)(=O)C1)Cc1cc(F)ccc1F] | 884/91 | — |
| 71 | 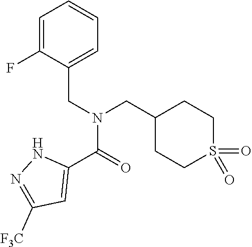 [SMILES: O=C(c1cc(C(F)(F)F)n[nH]1)N(Cc1ccccc1F)CC1CCS(=O)(=O)CC1] | 10200/71 | — |
| 72 | 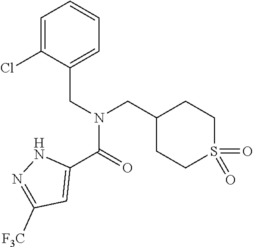 [SMILES: O=C(c1cc(C(F)(F)F)n[nH]1)N(Cc1ccccc1Cl)CC1CCS(=O)(=O)CC1] | 19660/22 | — |
| 73 | 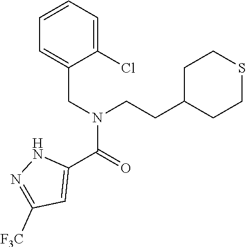 [SMILES: O=C(c1cc(C(F)(F)F)n[nH]1)N(CCC1CCSCC1)Cc1ccccc1Cl] | 1343/79 | — |
| 74 | 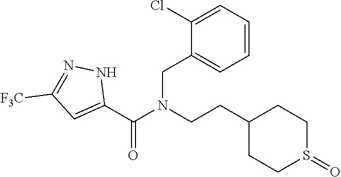 [SMILES: O=C(c1cc(C(F)(F)F)n[nH]1)N(CCC1CCS(=O)CC1)Cc1ccccc1Cl] | 640/95 | — |
| 75 | 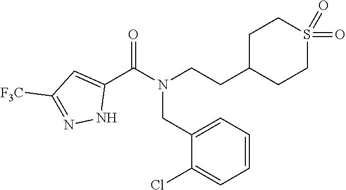 [SMILES: O=C(c1cc(C(F)(F)F)n[nH]1)N(CCC1CCS(=O)(=O)CC1)Cc1ccccc1Cl] | 188/102 | — |
| 76 | 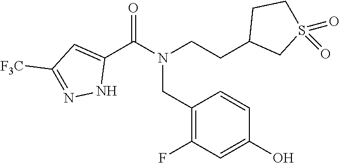 [SMILES: O=C(c1cc(C(F)(F)F)n[nH]1)N(CCC1CCS(=O)(=O)C1)Cc1ccc(O)cc1F] | 54/93 | — |
| 77 | 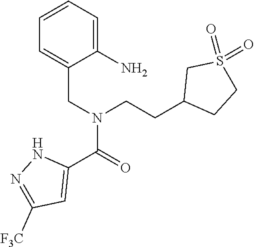 [SMILES: Nc1ccccc1CN(CCC1CCS(=O)(=O)C1)C(=O)c1cc(C(F)(F)F)n[nH]1] | 34820/43 | — |
| 78 | 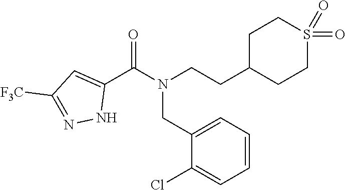 [SMILES: O=C(c1cc(C(F)(F)F)n[nH]1)N(CCC1CCS(=O)(=O)CC1)Cc1ccccc1Cl] | 485/102 | — |
| 79 | 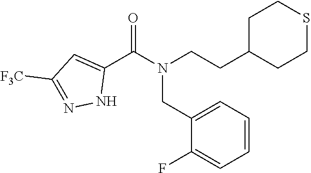 [SMILES: O=C(c1cc(C(F)(F)F)n[nH]1)N(CCC1CCSCC1)Cc1ccccc1F] | 1193/95 | — |
| 80 | 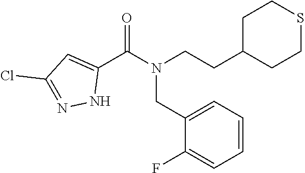 [SMILES: O=C(c1cc(Cl)n[nH]1)N(CCC1CCSCC1)Cc1ccccc1F] | 1301/46 | — |
| 81 | 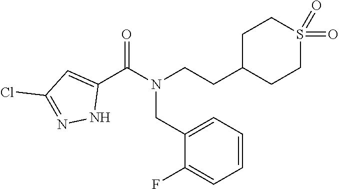 [SMILES: O=C(c1cc(Cl)n[nH]1)N(CCC1CCS(=O)(=O)CC1)Cc1ccccc1F] | 1366/72 | — |
| 82 | 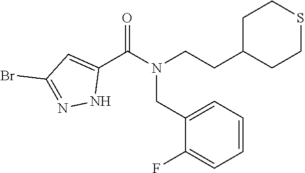 [SMILES: O=C(c1cc(Br)n[nH]1)N(CCC1CCSCC1)Cc1ccccc1F] | 1372/74 | — |
| 83 | 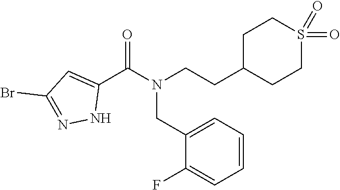 [SMILES: O=C(c1cc(Br)n[nH]1)N(CCC1CCS(=O)(=O)CC1)Cc1ccccc1F] | 1104/98 | — |
| 84 | 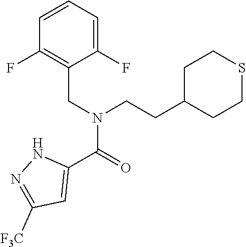 [SMILES: O=C(c1cc(C(F)(F)F)n[nH]1)N(CCC1CCSCC1)Cc1c(F)cccc1F] | 697/73 | — |
| 85 | 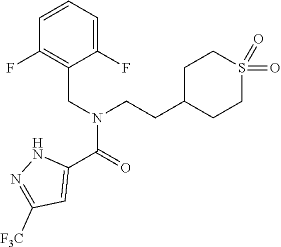 [SMILES: O=C(c1cc(C(F)(F)F)n[nH]1)N(CCC1CCS(=O)(=O)CC1)Cc1c(F)cccc1F] | 64/100 | — |
| 86 | 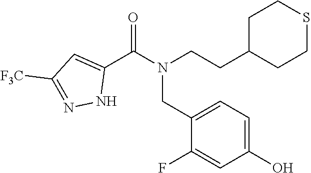 [SMILES: O=C(c1cc(C(F)(F)F)n[nH]1)N(CCC1CCSCC1)Cc1ccc(O)cc1F] | 93/98 | — |
| 87 | 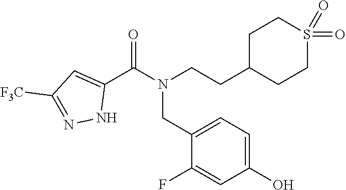 [SMILES: O=C(c1cc(C(F)(F)F)n[nH]1)N(CCC1CCS(=O)(=O)CC1)Cc1ccc(O)cc1F] | 64/100 | — |
| 88 | 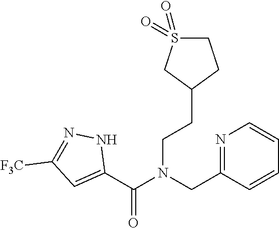 [SMILES: O=C(c1cc(C(F)(F)F)n[nH]1)N(CCC1CCS(=O)(=O)C1)Cc1ccccn1] | 3600/96 | TFA |
| 89 | 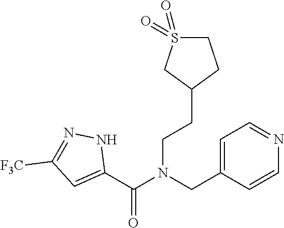 [SMILES: O=C(c1cc(C(F)(F)F)n[nH]1)N(CCC1CCS(=O)(=O)C1)Cc1ccncc1] | 58000/35 | TFA |
| 90 | 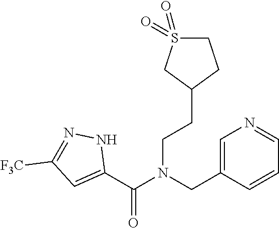 [SMILES: O=C(c1cc(C(F)(F)F)n[nH]1)N(CCC1CCS(=O)(=O)C1)Cc1cccnc1] | 52000/24 | TFA |
| 91 | 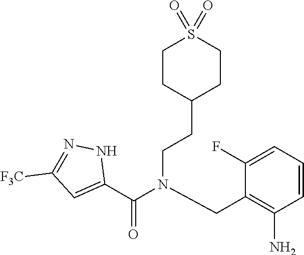 [SMILES: Nc1cccc(F)c1CN(CCC1CCS(=O)(=O)CC1)C(=O)c1cc(C(F)(F)F)n[nH]1] | 58/78 | TFA |
| 92 | 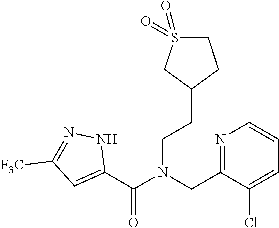 [SMILES: O=C(c1cc(C(F)(F)F)n[nH]1)N(CCC1CCS(=O)(=O)C1)Cc1ncccc1Cl] | 1290/74 | TFA |
| 93 | 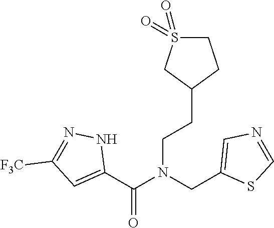 [SMILES: O=C(c1cc(C(F)(F)F)n[nH]1)N(CCC1CCS(=O)(=O)C1)Cc1cncs1] | 49000/26 | — |
| 94 | 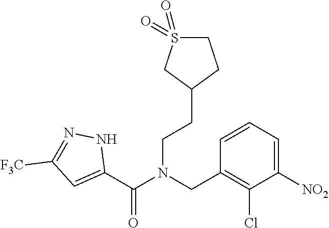 [SMILES: O=C(c1cc(C(F)(F)F)n[nH]1)N(CCC1CCS(=O)(=O)C1)Cc1cccc([N+](=O)[O-])c1Cl] | 2380/69 | — |
| 95 | 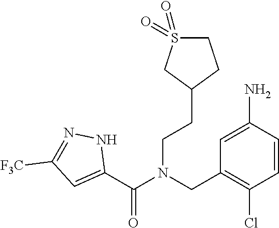 [SMILES: Nc1ccc(Cl)c(CN(CCC2CCS(=O)(=O)C2)C(=O)c2cc(C(F)(F)F)n[nH]2)c1] | TBD | TFA |
| 96 | 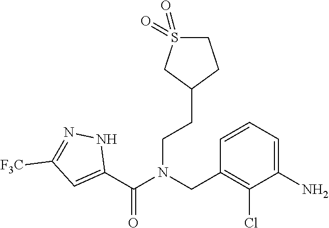 [SMILES: Nc1cccc(CN(CCC2CCS(=O)(=O)C2)C(=O)c2cc(C(F)(F)F)n[nH]2)c1Cl] | TBD | TFA |
| 97 | 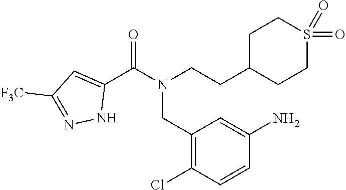 [SMILES: Nc1ccc(Cl)c(CN(CCC2CCS(=O)(=O)CC2)C(=O)c2cc(C(F)(F)F)n[nH]2)c1] | TBD | TFA |
| 98 | 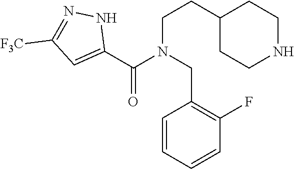 [SMILES: O=C(c1cc(C(F)(F)F)n[nH]1)N(CCC1CCNCC1)Cc1ccccc1F] | 2050/56 | TFA |
| 99 | 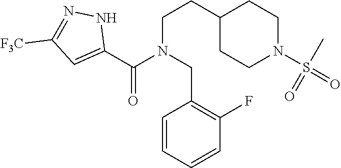 [SMILES: CS(=O)(=O)N1CCC(CCN(Cc2ccccc2F)C(=O)c2cc(C(F)(F)F)n[nH]2)CC1] | 110/100 | — |
| 100 | 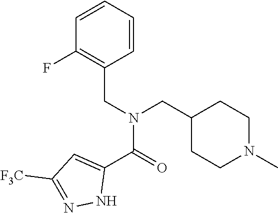 [SMILES: CN1CCC(CN(Cc2ccccc2F)C(=O)c2cc(C(F)(F)F)n[nH]2)CC1] | 101000/29 | TFA |
| 101 | 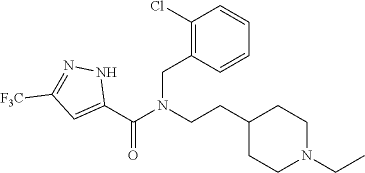 [SMILES: CCN1CCC(CCN(Cc2ccccc2Cl)C(=O)c2cc(C(F)(F)F)n[nH]2)CC1] | 1856/96 | TFA |
| 102 | 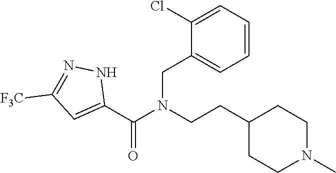 [SMILES: CN1CCC(CCN(Cc2ccccc2Cl)C(=O)c2cc(C(F)(F)F)n[nH]2)CC1] | 767/71 | TFA |
| 103 | 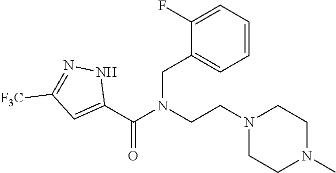 [SMILES: CN1CCN(CCN(Cc2ccccc2F)C(=O)c2cc(C(F)(F)F)n[nH]2)CC1] | 37000/42 | 2TFA |
| 104 | 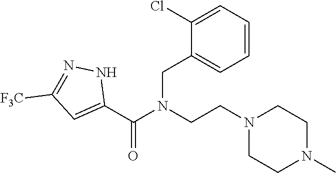 [SMILES: CN1CCN(CCN(Cc2ccccc2Cl)C(=O)c2cc(C(F)(F)F)n[nH]2)CC1] | 17000/63 | 2TFA |
| 105 | 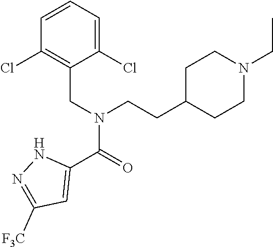 [SMILES: CCN1CCC(CCN(Cc2c(Cl)cccc2Cl)C(=O)c2cc(C(F)(F)F)n[nH]2)CC1] | 3268/71 | TFA |
| 106 | 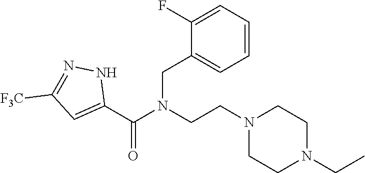 [SMILES: CCN1CCN(CCN(Cc2ccccc2F)C(=O)c2cc(C(F)(F)F)n[nH]2)CC1] | 6300/115 | 2TFA |
| 107 | 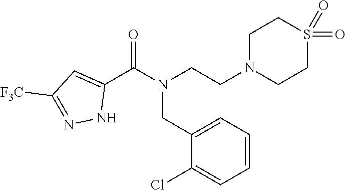 [SMILES: O=C(c1cc(C(F)(F)F)n[nH]1)N(CCN1CCS(=O)(=O)CC1)Cc1ccccc1Cl] | 873/85 | TFA |
| 108 | 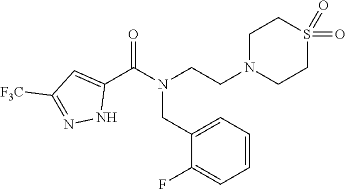 [SMILES: O=C(c1cc(C(F)(F)F)n[nH]1)N(CCN1CCS(=O)(=O)CC1)Cc1ccccc1F] | 7800/92 | TFA |
| 109 | 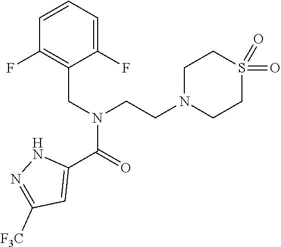 [SMILES: O=C(c1cc(C(F)(F)F)n[nH]1)N(CCN1CCS(=O)(=O)CC1)Cc1c(F)cccc1F] | 4200/94 | TFA |
| 110 | 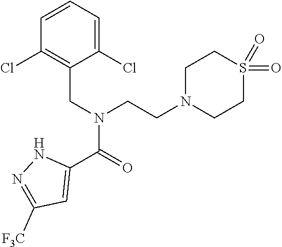 [SMILES: O=C(c1cc(C(F)(F)F)n[nH]1)N(CCN1CCS(=O)(=O)CC1)Cc1c(Cl)cccc1Cl] | 1644/84 | TFA |
| 111 | 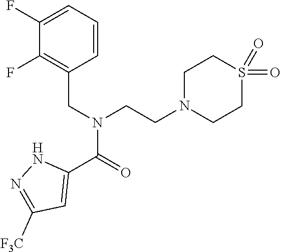 [SMILES: O=C(c1cc(C(F)(F)F)n[nH]1)N(CCN1CCS(=O)(=O)CC1)Cc1cccc(F)c1F] | 13100/64 | TFA |
| 112 | 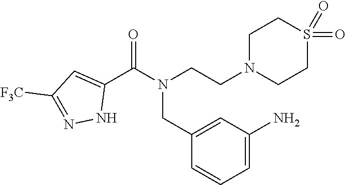 [SMILES: Nc1cccc(CN(CCN2CCS(=O)(=O)CC2)C(=O)c2cc(C(F)(F)F)n[nH]2)c1] | 1066/91 | 2TFA |
| 113 | 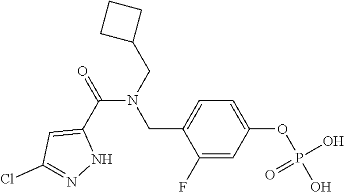 [SMILES: O=C(c1cc(Cl)n[nH]1)N(Cc1ccc(OP(=O)(O)O)cc1F)CC1CCC1] | 77000/21 | — |
| 114 | 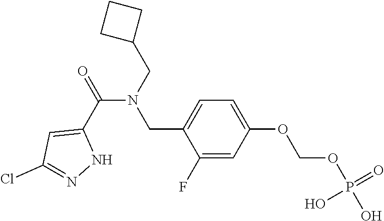 [SMILES: O=C(c1cc(Cl)n[nH]1)N(Cc1ccc(OCOP(=O)(O)O)cc1F)CC1CCC1] | TBD | — |
| 115 | 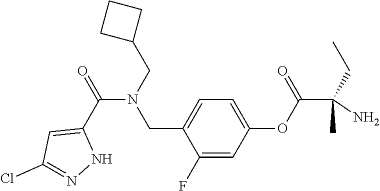 [SMILES: CC[C@](C)(N)C(=O)Oc1ccc(CN(CC2CCC2)C(=O)c2cc(Cl)n[nH]2)c(F)c1] | 98/104 | TFA |
| 116 | 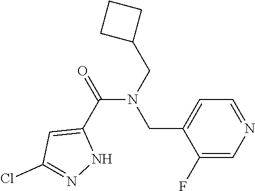 [SMILES: O=C(c1cc(Cl)n[nH]1)N(Cc1ccncc1F)CC1CCC1] | 38000/NA | TFA |
| 117 | 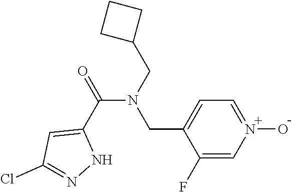 [SMILES: O=C(c1cc(Cl)n[nH]1)N(Cc1cc[n+](O)cc1F)CC1CCC1] | NA/NA | TFA |
| 118 | 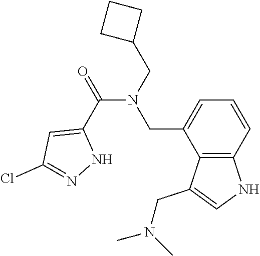 [SMILES: CN(C)Cc1c[nH]c2cccc(CN(CC3CCC3)C(=O)c3cc(Cl)n[nH]3)c12] | 7000/100 | TFA |
| 119 | 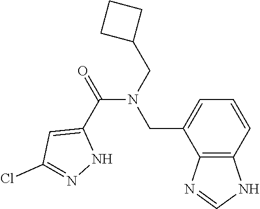 [SMILES: O=C(c1cc(Cl)n[nH]1)N(Cc1cccc2[nH]cnc12)CC1CCC1] | 2933/100 | — |
| 120 | 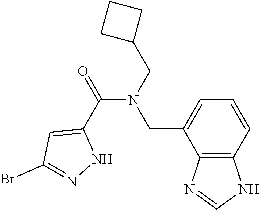 [SMILES: O=C(c1cc(Br)n[nH]1)N(Cc1cccc2[nH]cnc12)CC1CCC1] | 1335/100 | — |
| 121 | 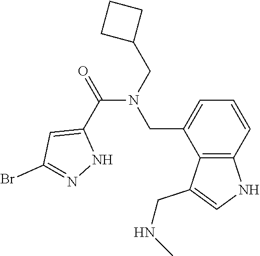 [SMILES: CNCc1c[nH]c2cccc(CN(CC3CCC3)C(=O)c3cc(Br)n[nH]3)c12] | 23,000/NA | TFA |
| 122 | 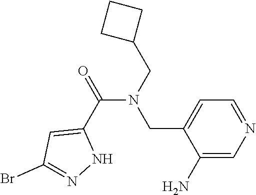 [SMILES: Nc1cnccc1CN(CC1CCC1)C(=O)c1cc(Br)n[nH]1] | NA/NA | — |
| 123 | 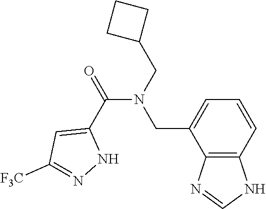 [SMILES: O=C(c1cc(C(F)(F)F)n[nH]1)N(Cc1cccc2[nH]cnc12)CC1CCC1] | 828/100 | — |
| 124 | 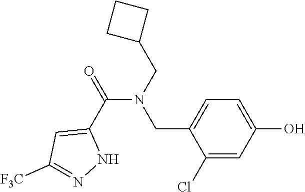 [SMILES: O=C(c1cc(C(F)(F)F)n[nH]1)N(Cc1ccc(O)cc1Cl)CC1CCC1] | 21/100 | — |
| 125 | 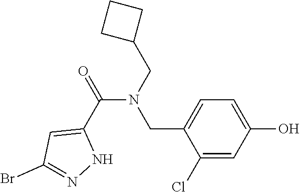 [SMILES: O=C(c1cc(Br)n[nH]1)N(Cc1ccc(O)cc1Cl)CC1CCC1] | 35/100 | — |
| 126 | 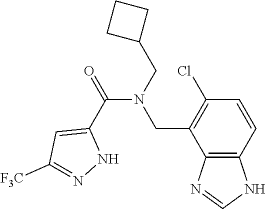 [SMILES: O=C(c1cc(C(F)(F)F)n[nH]1)N(Cc1c(Cl)ccc2[nH]cnc12)CC1CCC1] | NA/NA | — |
| 127 | 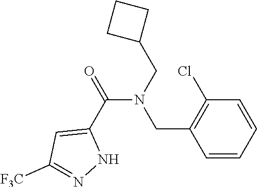 [SMILES: O=C(c1cc(C(F)(F)F)n[nH]1)N(Cc1ccccc1Cl)CC1CCC1] | 258/100 | — |
| 128 | 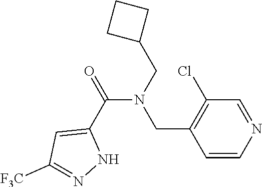 [SMILES: O=C(c1cc(C(F)(F)F)n[nH]1)N(Cc1ccncc1Cl)CC1CCC1] | 9300/75 | — |
| 129 | 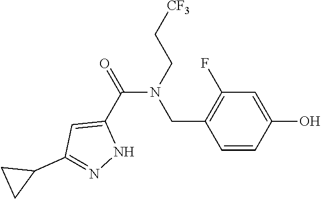 [SMILES: O=C(c1cc(C2CC2)n[nH]1)N(CCC(F)(F)F)Cc1ccc(O)cc1F] | 10500/NA | — |
| 130 | 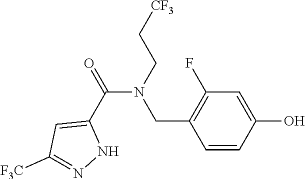 [SMILES: O=C(c1cc(C(F)(F)F)n[nH]1)N(CCC(F)(F)F)Cc1ccc(O)cc1F] | 1000/100 | — |
| 131 | 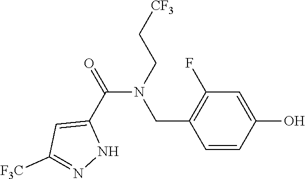 [SMILES: O=C(c1cc(C(F)(F)F)n[nH]1)N(CCC(F)(F)F)Cc1ccc(O)cc1F] | 23000/NA | — |
| 132 | 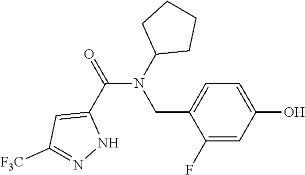 [SMILES: O=C(c1cc(C(F)(F)F)n[nH]1)N(Cc1ccc(O)cc1F)C1CCCC1] | 213/100 | — |
| 133 | 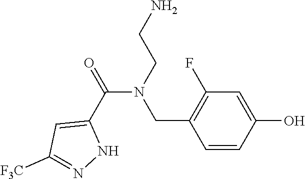 [SMILES: NCCN(Cc1ccc(O)cc1F)C(=O)c1cc(C(F)(F)F)n[nH]1] | 1270/100 | TFA |
| 134 | 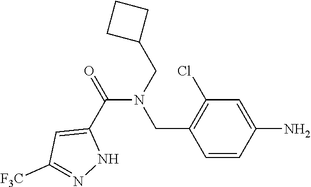 [SMILES: Nc1ccc(CN(CC2CCC2)C(=O)c2cc(C(F)(F)F)n[nH]2)c(Cl)c1] | 1075/100 | — |
| 135 | 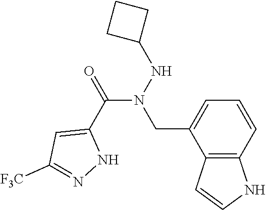 [SMILES: O=C(c1cc(C(F)(F)F)n[nH]1)N(Cc1cccc2[nH]ccc12)NC1CCC1] | 68/100 | TFA |
| 136 | 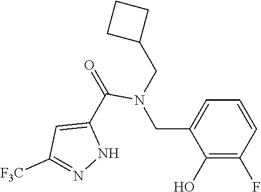 [SMILES: O=C(c1cc(C(F)(F)F)n[nH]1)N(Cc1cccc(F)c1O)CC1CCC1] | 380/100 | — |
| 137 | 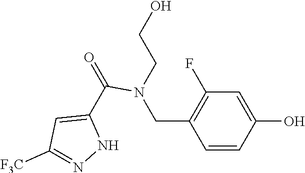 [SMILES: O=C(c1cc(C(F)(F)F)n[nH]1)N(CCO)Cc1ccc(O)cc1F] | 6200/70 | — |
| 138 | 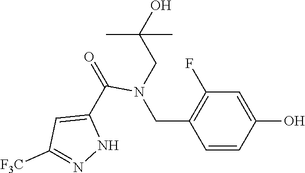 [SMILES: CC(C)(O)CN(Cc1ccc(O)cc1F)C(=O)c1cc(C(F)(F)F)n[nH]1] | NA/NA | — |
| 139 | 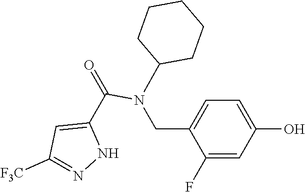 [SMILES: O=C(c1cc(C(F)(F)F)n[nH]1)N(Cc1ccc(O)cc1F)C1CCCCC1] | 152/100 | — |
| 140 | 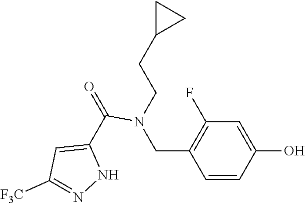 [SMILES: O=C(c1cc(C(F)(F)F)n[nH]1)N(CCC1CC1)Cc1ccc(O)cc1F] | 32/100 | — |
| 141 | 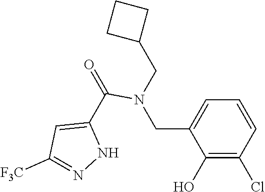 [SMILES: O=C(c1cc(C(F)(F)F)n[nH]1)N(Cc1cccc(Cl)c1O)CC1CCC1] | 6500/100 | — |
| 142 | 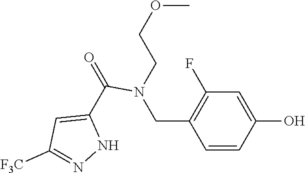 [SMILES: COCCN(Cc1ccc(O)cc1F)C(=O)c1cc(C(F)(F)F)n[nH]1] | 1190/100 | — |
| 143 | 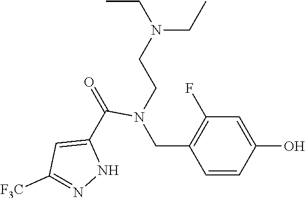 [SMILES: CCN(CC)CCN(Cc1ccc(O)cc1F)C(=O)c1cc(C(F)(F)F)n[nH]1] | 108/100 | TFA |
| 144 | 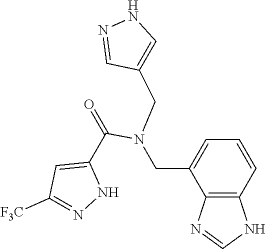 [SMILES: O=C(c1cc(C(F)(F)F)n[nH]1)N(Cc1cn[nH]c1)Cc1cccc2[nH]cnc12] | 25000/NA | TFA |
| 145 | 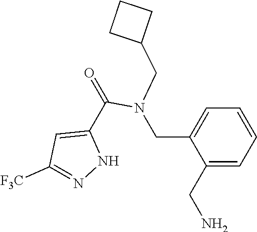 [SMILES: NCc1ccccc1CN(CC1CCC1)C(=O)c1cc(C(F)(F)F)n[nH]1] | 25500/NA | TFA |
| 146 | 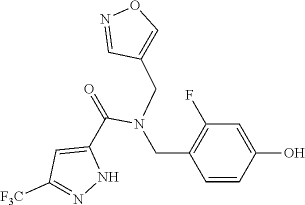 [SMILES: O=C(c1cc(C(F)(F)F)n[nH]1)N(Cc1cnoc1)Cc1ccc(O)cc1F] | NA/NA | TFA |
| 147 | 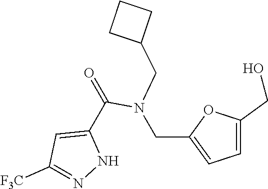 [SMILES: O=C(c1cc(C(F)(F)F)n[nH]1)N(Cc1ccc(CO)o1)CC1CCC1] | NA/NA | — |
| 148 | 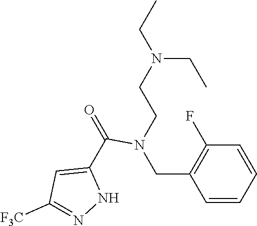 [SMILES: CCN(CC)CCN(Cc1ccccc1F)C(=O)c1cc(C(F)(F)F)n[nH]1] | 505/100 | TFA |
| 149 | 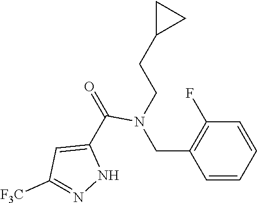 [SMILES: O=C(c1cc(C(F)(F)F)n[nH]1)N(CCC1CC1)Cc1ccccc1F] | 1995/100 | — |
| 150 | 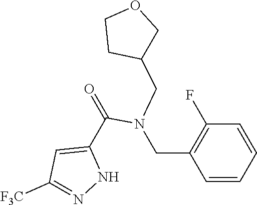 [SMILES: O=C(c1cc(C(F)(F)F)n[nH]1)N(Cc1ccccc1F)CC1CCOC1] | 16000/68 | — |
| 151 | 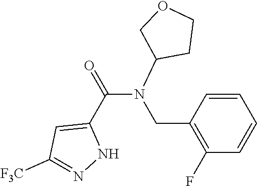 [SMILES: O=C(c1cc(C(F)(F)F)n[nH]1)N(Cc1ccccc1F)C1CCOC1] | NA/NA | — |
| 152 | 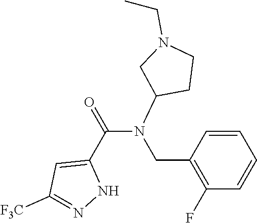 [SMILES: CCN1CCC(N(Cc2ccccc2F)C(=O)c2cc(C(F)(F)F)n[nH]2)C1] | 13500/68 | TFA |
| 153 | 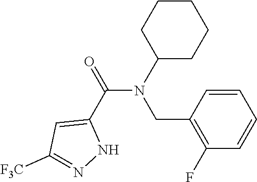 [SMILES: O=C(c1cc(C(F)(F)F)n[nH]1)N(Cc1ccccc1F)C1CCCCC1] | 19000/57 | — |
| 154 | 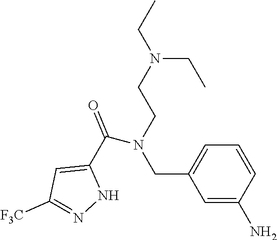 [SMILES: CCN(CC)CCN(Cc1cccc(N)c1)C(=O)c1cc(C(F)(F)F)n[nH]1] | 1430/100 | TFA |
| 155 | 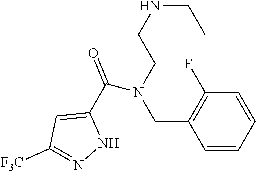 [SMILES: CCNCCN(Cc1ccccc1F)C(=O)c1cc(C(F)(F)F)n[nH]1] | 2150/100 | TFA |
| 156 | 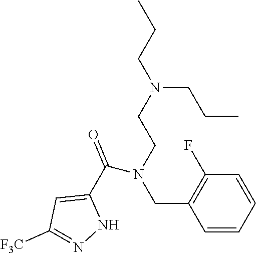 [SMILES: CCCN(CCC)CCN(Cc1ccccc1F)C(=O)c1cc(C(F)(F)F)n[nH]1] | NA/NA | TFA |
| 157 | 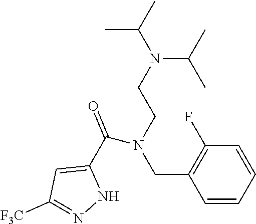 [SMILES: CC(C)N(CCN(Cc1ccccc1F)C(=O)c1cc(C(F)(F)F)n[nH]1)C(C)C] | NA/NA | TFA |
| 158 | 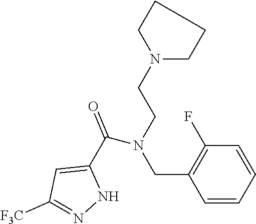 [SMILES: O=C(c1cc(C(F)(F)F)n[nH]1)N(CCN1CCCC1)Cc1ccccc1F] | 265/100 | TFA |
| 159 | 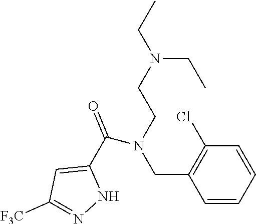 [SMILES: CCN(CC)CCN(Cc1ccccc1Cl)C(=O)c1cc(C(F)(F)F)n[nH]1] | 73/100 | TFA |
| 160 | 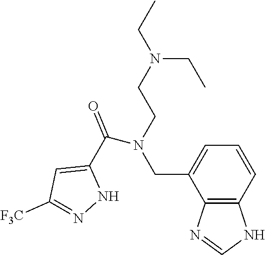 [SMILES: CCN(CC)CCN(Cc1cccc2[nH]cnc12)C(=O)c1cc(C(F)(F)F)n[nH]1] | 825/100 | TFA |
| 161 | 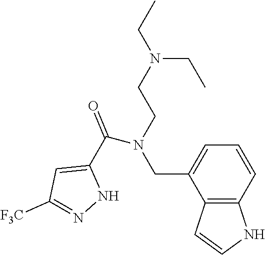 [SMILES: CCN(CC)CCN(Cc1cccc2[nH]ccc12)C(=O)c1cc(C(F)(F)F)n[nH]1] | 76/100 | TFA |
| 162 | 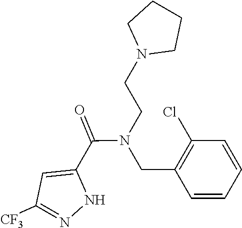 [SMILES: O=C(c1cc(C(F)(F)F)n[nH]1)N(CCN1CCCC1)Cc1ccccc1Cl] | 107/100 | TFA |
| 163 | 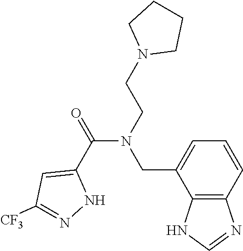 [SMILES: O=C(c1cc(C(F)(F)F)n[nH]1)N(CCN1CCCC1)Cc1cccc2nc[nH]c12] | 1085/100 | TFA |
| 164 | 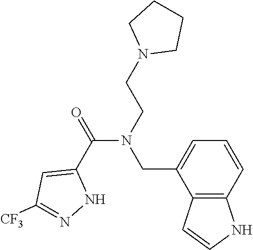 [SMILES: O=C(c1cc(C(F)(F)F)n[nH]1)N(CCN1CCCC1)Cc1cccc2[nH]ccc12] | 113/100 | TFA |
| 165 | 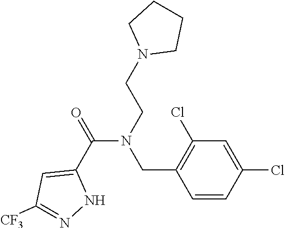 [SMILES: O=C(c1cc(C(F)(F)F)n[nH]1)N(CCN1CCCC1)Cc1ccc(Cl)cc1Cl] | 45000/50 | TFA |
| 166 | 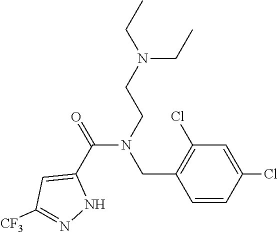 [SMILES: CCN(CC)CCN(Cc1ccc(Cl)cc1Cl)C(=O)c1cc(C(F)(F)F)n[nH]1] | NA/NA | TFA |
| 167 | 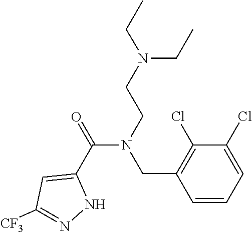 [SMILES: CCN(CC)CCN(Cc1cccc(Cl)c1Cl)C(=O)c1cc(C(F)(F)F)n[nH]1] | 186/100 | TFA |
| 168 | 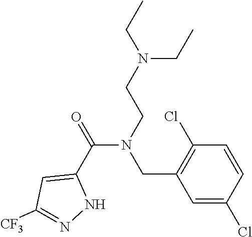 [SMILES: CCN(CC)CCN(Cc1cc(Cl)ccc1Cl)C(=O)c1cc(C(F)(F)F)n[nH]1] | 97/100 | TFA |
| 169 | 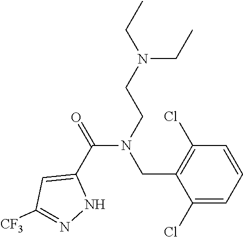 [SMILES: CCN(CC)CCN(Cc1c(Cl)cccc1Cl)C(=O)c1cc(C(F)(F)F)n[nH]1] | 66/100 | TFA |
| 170 | 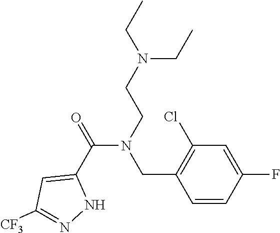 [SMILES: CCN(CC)CCN(Cc1ccc(F)cc1Cl)C(=O)c1cc(C(F)(F)F)n[nH]1] | 505/100 | TFA |
| 171 | 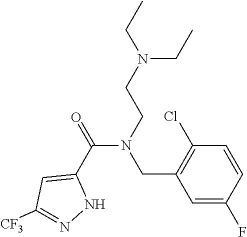 [SMILES: CCN(CC)CCN(Cc1cc(F)ccc1Cl)C(=O)c1cc(C(F)(F)F)n[nH]1] | 88/100 | TFA |
| 172 | 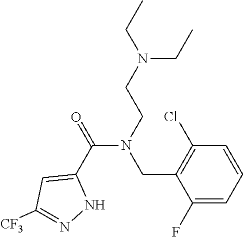 [SMILES: CCN(CC)CCN(Cc1c(F)cccc1Cl)C(=O)c1cc(C(F)(F)F)n[nH]1] | 94/100 | TFA |
| 173 | 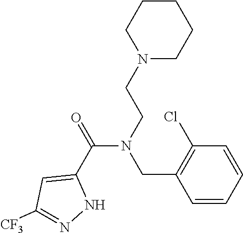 [SMILES: O=C(c1cc(C(F)(F)F)n[nH]1)N(CCN1CCCCC1)Cc1ccccc1Cl] | 66/100 | TFA |
| 174 | 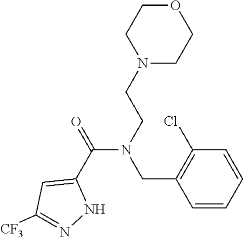 [SMILES: O=C(c1cc(C(F)(F)F)n[nH]1)N(CCN1CCOCC1)Cc1ccccc1Cl] | 1495/100 | TFA |
| 175 | 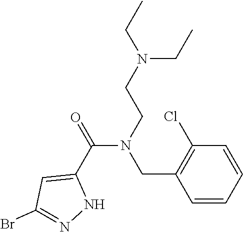 [SMILES: CCN(CC)CCN(Cc1ccccc1Cl)C(=O)c1cc(Br)n[nH]1] | 305/100 | TFA |
| 176 | 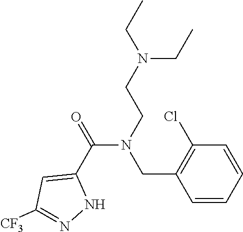 [SMILES: CCN(CC)CCN(Cc1ccccc1Cl)C(=O)c1cc(C(F)(F)F)n[nH]1] | 1150/100 | TFA |
| 177 | 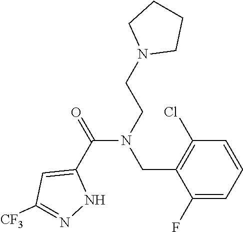 [SMILES: O=C(c1cc(C(F)(F)F)n[nH]1)N(CCN1CCCC1)Cc1c(F)cccc1Cl] | 180/100 | TFA |
| 178 | 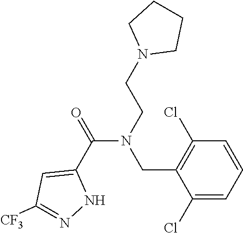 [SMILES: O=C(c1cc(C(F)(F)F)n[nH]1)N(CCN1CCCC1)Cc1c(Cl)cccc1Cl] | 126/100 | TFA |
| 179 | 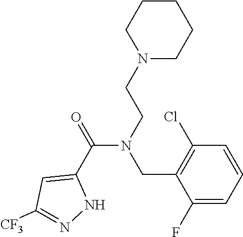 [SMILES: O=C(c1cc(C(F)(F)F)n[nH]1)N(CCN1CCCCC1)Cc1c(F)cccc1Cl] | 133/100 | TFA |
| 180 | 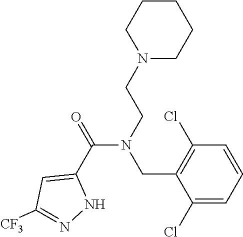 [SMILES: O=C(c1cc(C(F)(F)F)n[nH]1)N(CCN1CCCCC1)Cc1c(Cl)cccc1Cl] | 154/100 | TFA |
| 181 | 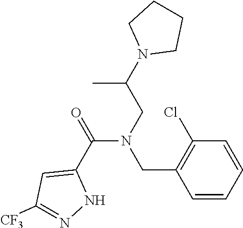 [SMILES: CC(CN(Cc1ccccc1Cl)C(=O)c1cc(C(F)(F)F)n[nH]1)N1CCCC1] | 430/100 | TFA |
| 182 | 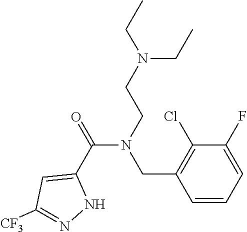 [SMILES: CCN(CC)CCN(Cc1cccc(F)c1Cl)C(=O)c1cc(C(F)(F)F)n[nH]1] | 109/100 | TFA |
| 183 | 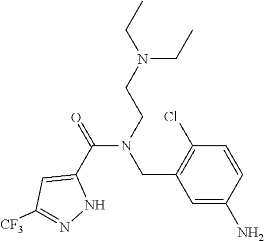 [SMILES: CCN(CC)CCN(Cc1cc(N)ccc1Cl)C(=O)c1cc(C(F)(F)F)n[nH]1] | 185/100 | TFA |
| 184 | 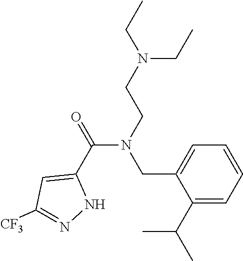 [SMILES: CCN(CC)CCN(Cc1ccccc1C(C)C)C(=O)c1cc(C(F)(F)F)n[nH]1] | 1950/85 | TFA |
| 185 | 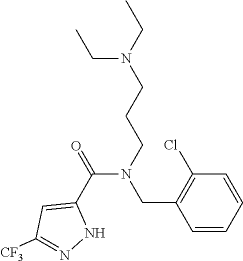 [SMILES: CCN(CC)CCCN(Cc1ccccc1Cl)C(=O)c1cc(C(F)(F)F)n[nH]1] | 1750/53 | TFA |
| 186 | 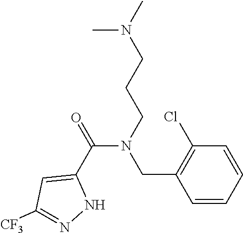 [SMILES: CN(C)CCCN(Cc1ccccc1Cl)C(=O)c1cc(C(F)(F)F)n[nH]1] | 375/100 | TFA |
| 187 | 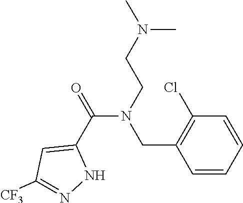 [SMILES: CN(C)CCN(Cc1ccccc1Cl)C(=O)c1cc(C(F)(F)F)n[nH]1] | 295/100 | TFA |
| 188 | 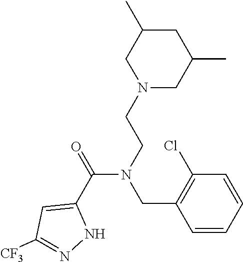 [SMILES: CC1CC(C)CN(CCN(Cc2ccccc2Cl)C(=O)c2cc(C(F)(F)F)n[nH]2)C1] | 46/60 | TFA |
| 189 | 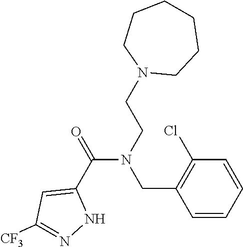 [SMILES: O=C(c1cc(C(F)(F)F)n[nH]1)N(CCN1CCCCCC1)Cc1ccccc1Cl] | 24/100 | TFA |
| 190 | 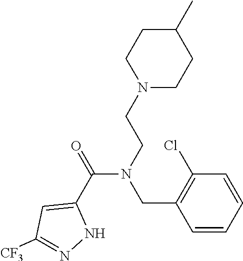 [SMILES: CC1CCN(CCN(Cc2ccccc2Cl)C(=O)c2cc(C(F)(F)F)n[nH]2)CC1] | 42/100 | TFA |
| 191 | 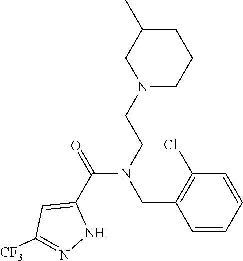 [SMILES: CC1CCCN(CCN(Cc2ccccc2Cl)C(=O)c2cc(C(F)(F)F)n[nH]2)C1] | 29/100 | TFA |
| 192 | 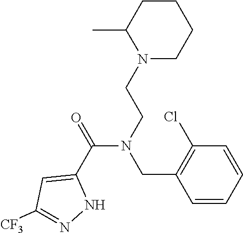 [SMILES: CC1CCCCN1CCN(Cc1ccccc1Cl)C(=O)c1cc(C(F)(F)F)n[nH]1] | 104/100 | TFA |
| 193 | 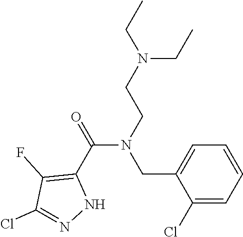 [SMILES: CCN(CC)CCN(Cc1ccccc1Cl)C(=O)c1[nH]nc(Cl)c1F] | 14000/58 | TFA |
| 194 | 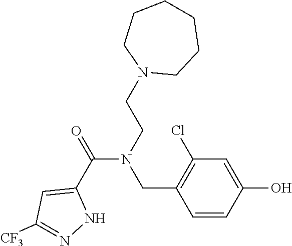 [SMILES: O=C(c1cc(C(F)(F)F)n[nH]1)N(CCN1CCCCCC1)Cc1ccc(O)cc1Cl] | 43/100 | TFA |
| 195 | 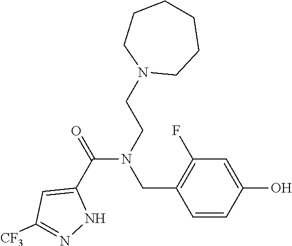 [SMILES: O=C(c1cc(C(F)(F)F)n[nH]1)N(CCN1CCCCCC1)Cc1ccc(O)cc1F] | 95/100 | TFA |
| 196 | 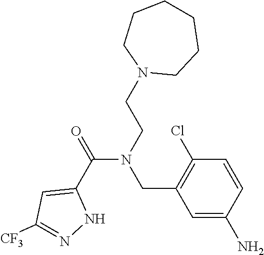 [SMILES: Nc1ccc(Cl)c(CN(CCN2CCCCCC2)C(=O)c2cc(C(F)(F)F)n[nH]2)c1] | 99/100 | TFA |
| 197 | 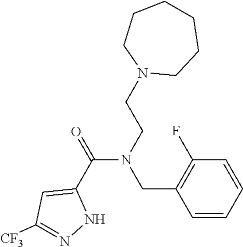 [SMILES: O=C(c1cc(C(F)(F)F)n[nH]1)N(CCN1CCCCCC1)Cc1ccccc1F] | 63/100 | TFA |
| 198 | 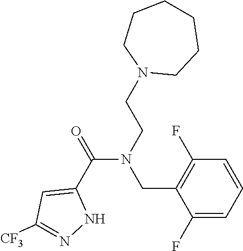 [SMILES: O=C(c1cc(C(F)(F)F)n[nH]1)N(CCN1CCCCCC1)Cc1c(F)cccc1F] | 130/100 | TFA |
| 199 | 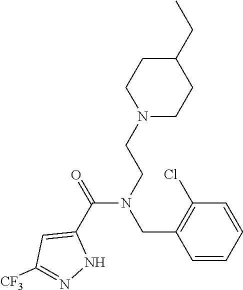 [SMILES: CCC1CCN(CCN(Cc2ccccc2Cl)C(=O)c2cc(C(F)(F)F)n[nH]2)CC1] | 100/100 | TFA |
| 200 | 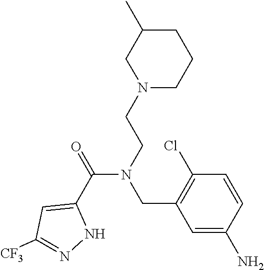 [SMILES: CC1CCCN(CCN(Cc2cc(N)ccc2Cl)C(=O)c2cc(C(F)(F)F)n[nH]2)C1] | 130/100 | TFA |
| 201 | 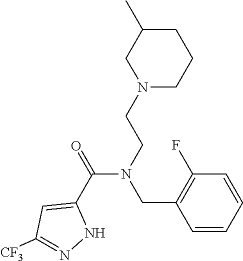 [SMILES: CC1CCCN(CCN(Cc2ccccc2F)C(=O)c2cc(C(F)(F)F)n[nH]2)C1] | 215/100 | TFA |
| 202 | 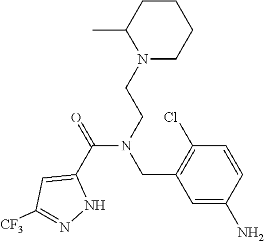 [SMILES: CC1CCCCN1CCN(Cc1cc(N)ccc1Cl)C(=O)c1cc(C(F)(F)F)n[nH]1] | 395/100 | TFA |
| 203 | 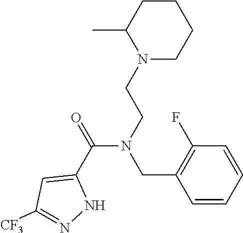 [SMILES: CC1CCCCN1CCN(Cc1ccccc1F)C(=O)c1cc(C(F)(F)F)n[nH]1] | 550/100 | TFA |
| 204 | 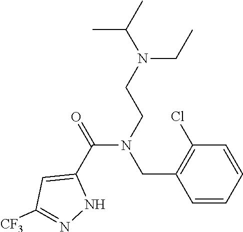 [SMILES: CCN(CCN(Cc1ccccc1Cl)C(=O)c1cc(C(F)(F)F)n[nH]1)C(C)C] | 880/53 | TFA |
| 205 | 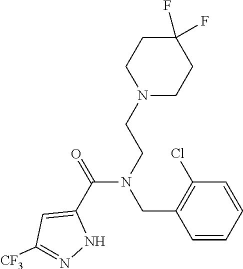 [SMILES: O=C(c1cc(C(F)(F)F)n[nH]1)N(CCN1CCC(F)(F)CC1)Cc1ccccc1Cl] | 2050/100 | TFA |
| 206 | 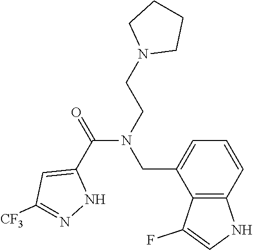 [SMILES: O=C(c1cc(C(F)(F)F)n[nH]1)N(CCN1CCCC1)Cc1cccc2[nH]cc(F)c12] | 136/100 | TFA |
| 207 | 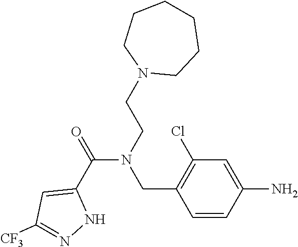 [SMILES: Nc1ccc(CN(CCN2CCCCCC2)C(=O)c2cc(C(F)(F)F)n[nH]2)c(Cl)c1] | 170/75 | TFA |
| 208 | 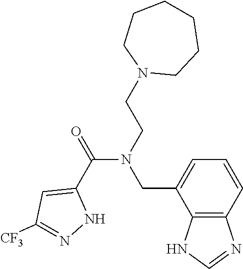 [SMILES: O=C(c1cc(C(F)(F)F)n[nH]1)N(CCN1CCCCCC1)Cc1cccc2nc[nH]c12] | 125/100 | TFA |
| 209 | 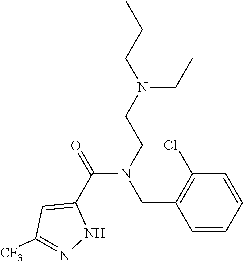 [SMILES: CCCN(CC)CCN(Cc1ccccc1Cl)C(=O)c1cc(C(F)(F)F)n[nH]1] | 52/100 | TFA |
| 210 | 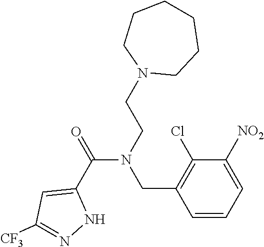 [SMILES: O=C(c1cc(C(F)(F)F)n[nH]1)N(CCN1CCCCCC1)Cc1cccc([N+](=O)[O-])c1Cl] | 133/100 | TFA |
| 211 | 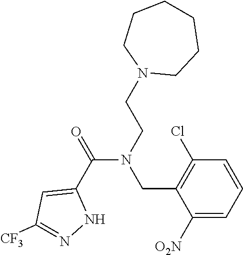 [SMILES: O=C(c1cc(C(F)(F)F)n[nH]1)N(CCN1CCCCCC1)Cc1c(Cl)cccc1[N+](=O)[O-]] | 123/100 | TFA |
| 212 | 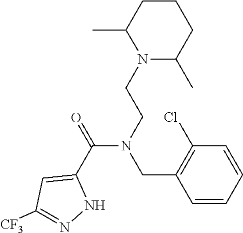 [SMILES: CC1CCCC(C)N1CCN(Cc1ccccc1Cl)C(=O)c1cc(C(F)(F)F)n[nH]1] | 5812/53 | TFA |
| 213 | 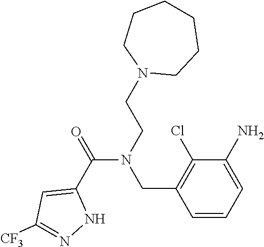 [SMILES: Nc1cccc(CN(CCN2CCCCCC2)C(=O)c2cc(C(F)(F)F)n[nH]2)c1Cl] | 60/100 | TFA |
| 214 | 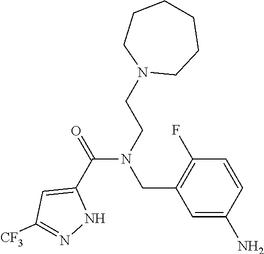 [SMILES: Nc1ccc(F)c(CN(CCN2CCCCCC2)C(=O)c2cc(C(F)(F)F)n[nH]2)c1] | 153/100 | TFA |
| 215 | 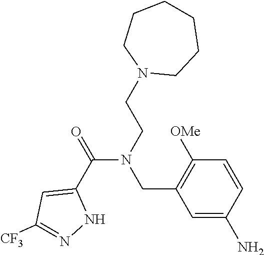 [SMILES: COc1ccc(N)cc1CN(CCN1CCCCCC1)C(=O)c1cc(C(F)(F)F)n[nH]1] | 1185/100 | TFA |
| 216 | 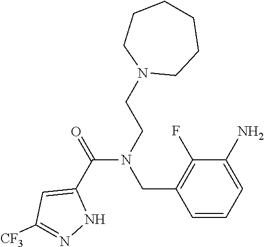 [SMILES: Nc1cccc(CN(CCN2CCCCCC2)C(=O)c2cc(C(F)(F)F)n[nH]2)c1F] | 117/100 | TFA |
| 217 | 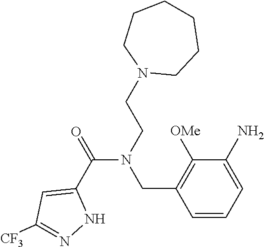 [SMILES: COc1c(N)cccc1CN(CCN1CCCCCC1)C(=O)c1cc(C(F)(F)F)n[nH]1] | 230/100 | TFA |
| 218 | 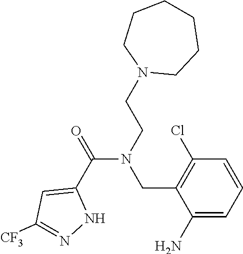 [SMILES: Nc1cccc(Cl)c1CN(CCN1CCCCCC1)C(=O)c1cc(C(F)(F)F)n[nH]1] | 41/100 | TFA |
| 219 | 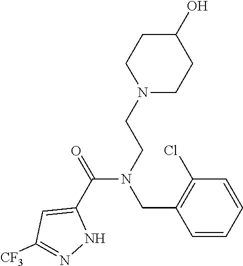 [SMILES: O=C(c1cc(C(F)(F)F)n[nH]1)N(CCN1CCC(O)CC1)Cc1ccccc1Cl] | 1300/100 | TFA |
| 220 | 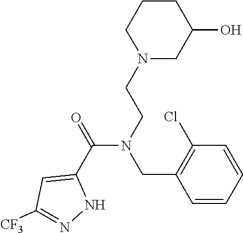 [SMILES: O=C(c1cc(C(F)(F)F)n[nH]1)N(CCN1CCCC(O)C1)Cc1ccccc1Cl] | 465/100 | TFA |
| 221 | 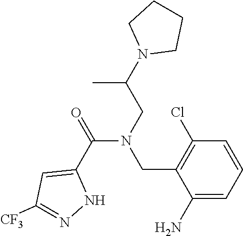 [SMILES: CC(CN(Cc1c(N)cccc1Cl)C(=O)c1cc(C(F)(F)F)n[nH]1)N1CCCC1] | 223/60 | TFA |
| 222 | 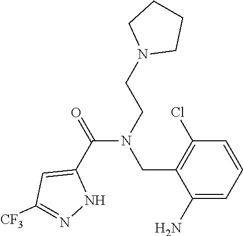 [SMILES: Nc1cccc(Cl)c1CN(CCN1CCCC1)C(=O)c1cc(C(F)(F)F)n[nH]1] | 56/100 | TFA |
| 223 | 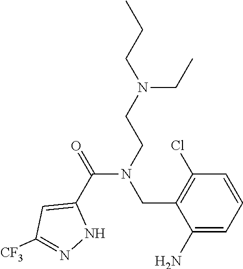 [SMILES: CCCN(CC)CCN(Cc1c(N)cccc1Cl)C(=O)c1cc(C(F)(F)F)n[nH]1] | 32/100 | TFA |
| 224 | 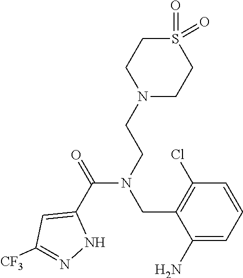 [SMILES: Nc1cccc(Cl)c1CN(CCN1CCS(=O)(=O)CC1)C(=O)c1cc(C(F)(F)F)n[nH]1] | 410/100 | TFA |
| 225 | 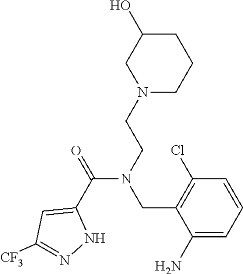 [SMILES: Nc1cccc(Cl)c1CN(CCN1CCCC(O)C1)C(=O)c1cc(C(F)(F)F)n[nH]1] | 102/100 | TFA |
| 226 | 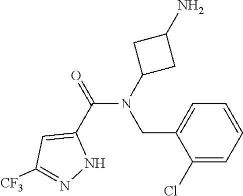 [SMILES: NC1CC(N(Cc2ccccc2Cl)C(=O)c2cc(C(F)(F)F)n[nH]2)C1] | 12000/80 | TFA |
| 227 | 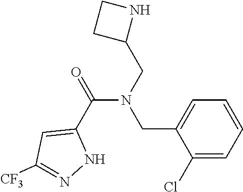 [SMILES: O=C(c1cc(C(F)(F)F)n[nH]1)N(Cc1ccccc1Cl)CC1CCN1] | 215/100 | TFA |
| 228 | 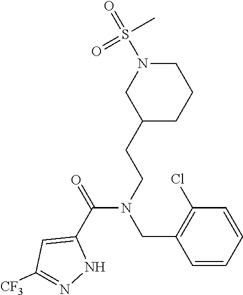 [SMILES: CS(=O)(=O)N1CCCC(CCN(Cc2ccccc2Cl)C(=O)c2cc(C(F)(F)F)n[nH]2)C1] | 2000/100 | TFA |
| 229 | 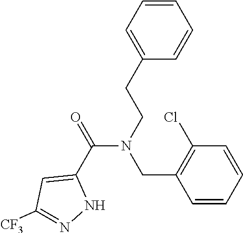 [SMILES: O=C(c1cc(C(F)(F)F)n[nH]1)N(CCc1ccccc1)Cc1ccccc1Cl] | NA/NA | — |
| 230 | 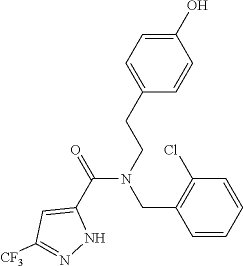 [SMILES: O=C(c1cc(C(F)(F)F)n[nH]1)N(CCc1ccc(O)cc1)Cc1ccccc1Cl] | 12000/100 | — |
| 231 | 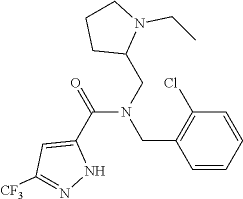 [SMILES: CCN1CCCC1CN(Cc1ccccc1Cl)C(=O)c1cc(C(F)(F)F)n[nH]1] | | |
| 232 | 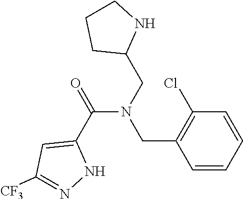 [SMILES: O=C(c1cc(C(F)(F)F)n[nH]1)N(Cc1ccccc1Cl)CC1CCCN1] | 320/85 | TFA |
| 233 | 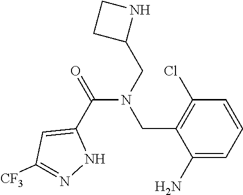 [SMILES: Nc1cccc(Cl)c1CN(CC1CCN1)C(=O)c1cc(C(F)(F)F)n[nH]1] | 930/75 | TFA |
| 234 | 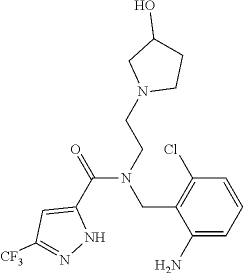 [SMILES: Nc1cccc(Cl)c1CN(CCN1CCC(O)C1)C(=O)c1cc(C(F)(F)F)n[nH]1] | 111/100 | TFA |
| 235 | 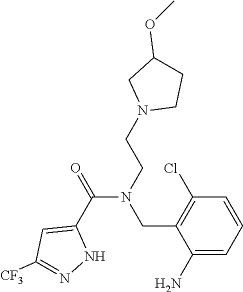 [SMILES: COC1CCN(CCN(Cc2c(N)cccc2Cl)C(=O)c2cc(C(F)(F)F)n[nH]2)C1] | 133/100 | TFA |
| 236 | 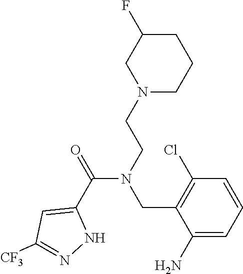 [SMILES: Nc1cccc(Cl)c1CN(CCN1CCCC(F)C1)C(=O)c1cc(C(F)(F)F)n[nH]1] | 160/73 | TFA |
| 237 | 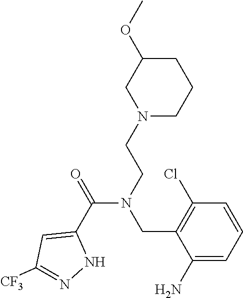 [SMILES: COC1CCCN(CCN(Cc2c(N)cccc2Cl)C(=O)c2cc(C(F)(F)F)n[nH]2)C1] | 183/100 | TFA |
| 238 | 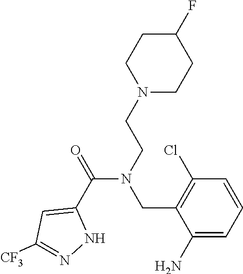 [SMILES: Nc1cccc(Cl)c1CN(CCN1CCC(F)CC1)C(=O)c1cc(C(F)(F)F)n[nH]1] | 116/100 | TFA |
| 239 | 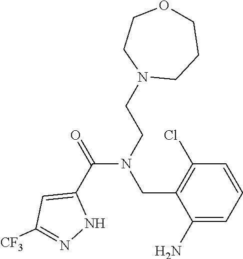 [SMILES: Nc1cccc(Cl)c1CN(CCN1CCCOCC1)C(=O)c1cc(C(F)(F)F)n[nH]1] | 115/100 | TFA |
| 240 | 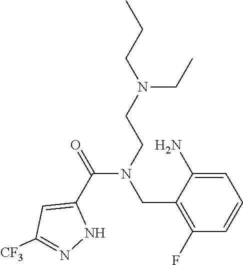 [SMILES: CCCN(CC)CCN(Cc1c(N)cccc1F)C(=O)c1cc(C(F)(F)F)n[nH]1] | 68/100 | TFA |
| 241 | 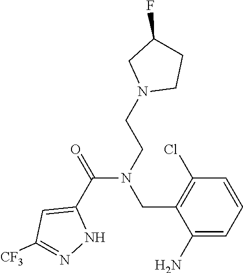 [SMILES: Nc1cccc(Cl)c1CN(CCN1CC[C@H](F)C1)C(=O)c1cc(C(F)(F)F)n[nH]1] | 125/100 | TFA |
| 242 | 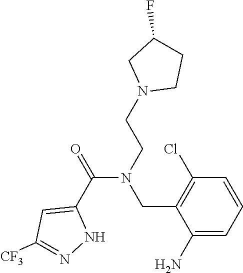 [SMILES: Nc1cccc(Cl)c1CN(CCN1CC[C@@H](F)C1)C(=O)c1cc(C(F)(F)F)n[nH]1] | 465/100 | TFA |
| 243 | 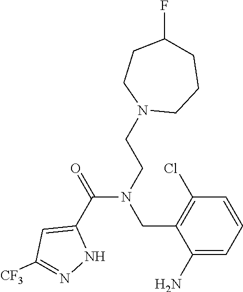 [SMILES: Nc1cccc(Cl)c1CN(CCN1CCCC(F)CC1)C(=O)c1cc(C(F)(F)F)n[nH]1] | 106/100 | TFA |
| 244 | 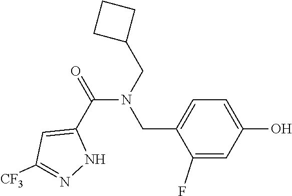 [SMILES: O=C(c1cc(C(F)(F)F)n[nH]1)N(Cc1ccc(O)cc1F)CC1CCC1] | 62/107 | — |
| 245 | 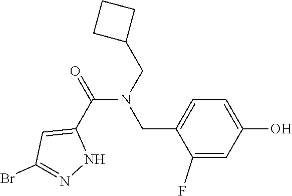 [SMILES: O=C(c1cc(Br)n[nH]1)N(Cc1ccc(O)cc1F)CC1CCC1] | 35/100 | — |
| 246 | 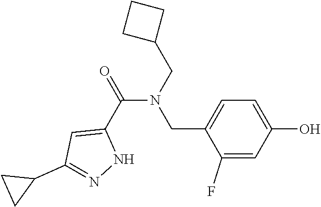 [SMILES: O=C(c1cc(C2CC2)n[nH]1)N(Cc1ccc(O)cc1F)CC1CCC1] | 92/85 | — |
| DASA | 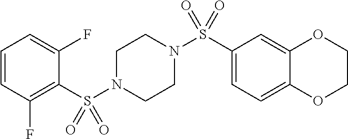 [SMILES: O=S(=O)(c1ccc2c(c1)OCCO2)N1CCN(S(=O)(=O)c2c(F)cccc2F)CC1] | — | — |
| DASA-58 | 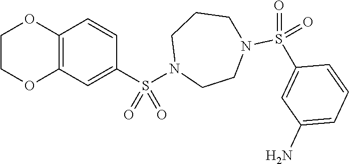 [SMILES: Nc1cccc(S(=O)(=O)N2CCCN(S(=O)(=O)c3ccc4c(c3)OCCO4)CC2)c1] | — | — |
| TEPP-46 | 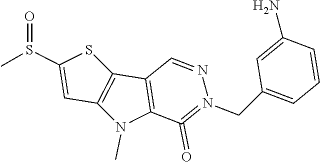 | | |
| PP1 | 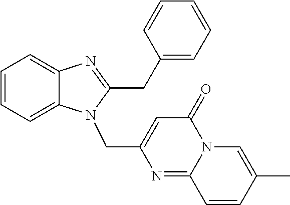 [SMILES: Cc1ccc2nc(Cn3c(Cc4ccccc4)nc4ccccc43)cc(=O)n2c1] | 3500/148 | |
| PP2 | 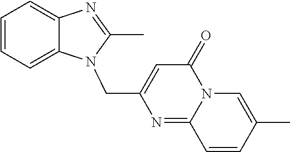 [SMILES: Cc1ccc2nc(Cn3c(C)nc4ccccc43)cc(=O)n2c1] | 10000/73 | |
| PP3 | 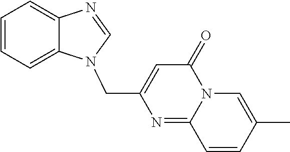 [SMILES: Cc1ccc2nc(Cn3cnc4ccccc43)cc(=O)n2c1] | 755/149 | |
| PP4 | 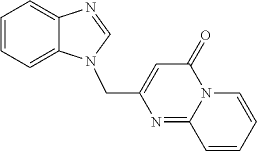 [SMILES: O=c1cc(Cn2cnc3ccccc32)nc2ccccn12] | 340/161 | |
| PP8 | 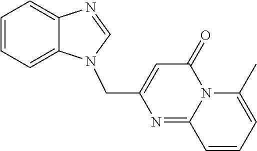 [SMILES: Cc1cccc2nc(Cn3cnc4ccccc43)cc(=O)n12] | 159/— | |
| PP9 | 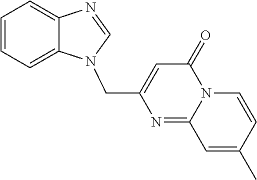 [SMILES: Cc1ccn2c(=O)cc(Cn3cnc4ccccc43)nc2c1] | 216/— | |
| PP10 | 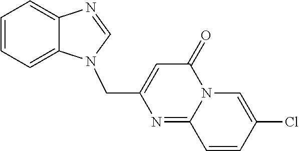 [SMILES: O=c1cc(Cn2cnc3ccccc32)nc2ccc(Cl)cn12] | 219/— | |
| PP11 | 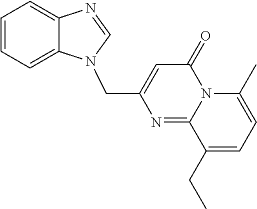 [SMILES: CCc1ccc(C)n2c(=O)cc(Cn3cnc4ccccc43)nc12] | 120/— | |
| PP12 | 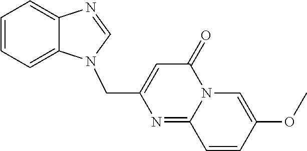 [SMILES: COc1ccc2nc(Cn3cnc4ccccc43)cc(=O)n2c1] | 470/— | |
| PP13 | 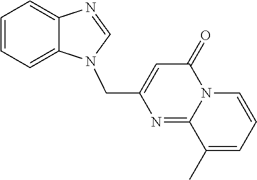 [SMILES: Cc1cccn2c(=O)cc(Cn3cnc4ccccc43)nc12] | 145/— | |
| PP14 | 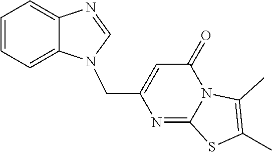 [SMILES: Cc1sc2nc(Cn3cnc4ccccc43)cc(=O)n2c1C] | 86/— | |
| PP15 | 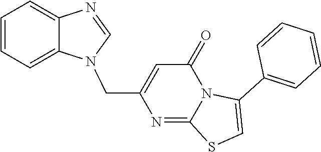 [SMILES: O=c1cc(Cn2cnc3ccccc32)nc2scc(-c3ccccc3)n12] | 1030/— | |
| PP16 | 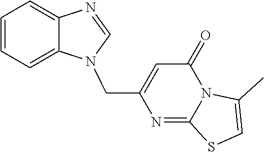 [SMILES: Cc1csc2nc(Cn3cnc4ccccc43)cc(=O)n12] | 127/— | |
| PP17 | 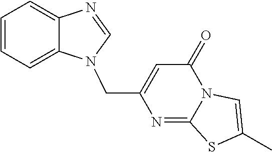 [SMILES: Cc1cn2c(=O)cc(Cn3cnc4ccccc43)nc2s1] | 466/— | |
| PP18 | 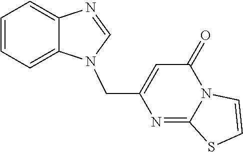 [SMILES: O=c1cc(Cn2cnc3ccccc32)nc2sccn12] | 266/— | |
| PP19 | 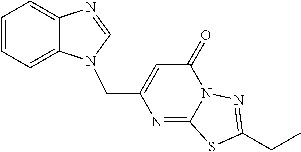 [SMILES: CCc1nn2c(=O)cc(Cn3cnc4ccccc43)nc2s1] | 4760/— | |
| PP20 | 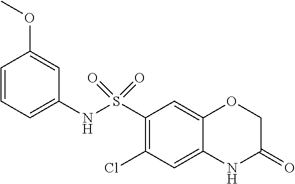 [SMILES: COc1cccc(NS(=O)(=O)c2cc3c(cc2Cl)NC(=O)CO3)c1] | 153/108 | |
| PMA8 | 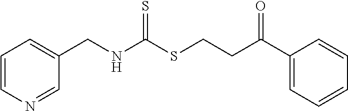 [SMILES: O=C(CCSC(=S)NCc1cccnc1)c1ccccc1] | 7640/24 | |
| PMA9 | 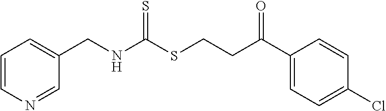 [SMILES: O=C(CCSC(=S)NCc1cccnc1)c1ccc(Cl)cc1] | >20000/— | |
| PMA10 | 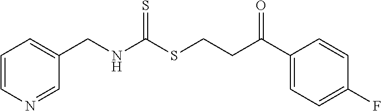 [SMILES: O=C(CCSC(=S)NCc1cccnc1)c1ccc(F)cc1] | 9830/48 | |
| PMA11 | 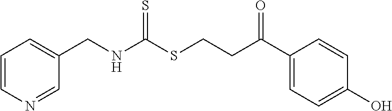 [SMILES: O=C(CCSC(=S)NCc1cccnc1)c1ccc(O)cc1] | 6230/76 | |
| PMA12 | 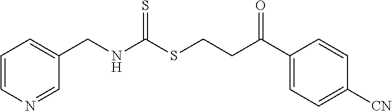 [SMILES: N#Cc1ccc(C(=O)CCSC(=S)NCc2cccnc2)cc1] | 4390/61 | |
| PMA13 | 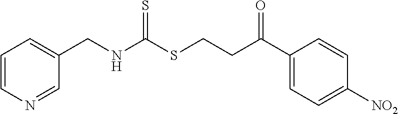 [SMILES: O=C(CCSC(=S)NCc1cccnc1)c1ccc([N+](=O)[O-])cc1] | 2760/49 | |
| PMA14 | 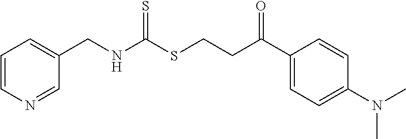 [SMILES: CN(C)c1ccc(C(=O)CCSC(=S)NCc2cccnc2)cc1] | — | |
| PMA15 | 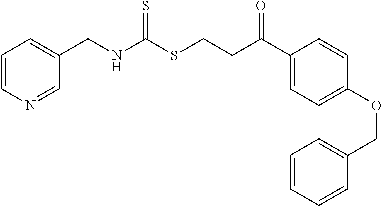 [SMILES: O=C(CCSC(=S)NCc1cccnc1)c1ccc(OCc2ccccc2)cc1] | >20000/— | |
| PMA16 | 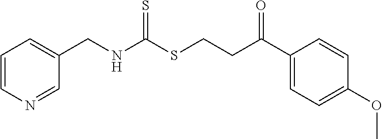 [SMILES: COc1ccc(C(=O)CCSC(=S)NCc2cccnc2)cc1] | 11180/63 | |
| PMA17 | 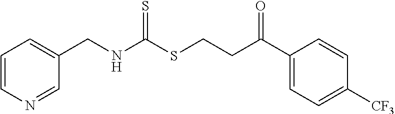 [SMILES: O=C(CCSC(=S)NCc1cccnc1)c1ccc(C(F)(F)F)cc1] | >20000/23 | |
| PMA18 | 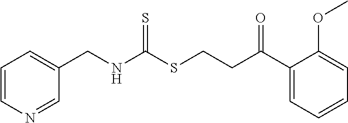 [SMILES: COc1ccccc1C(=O)CCSC(=S)NCc1cccnc1] | 3930/88 | |
| PMA19 | 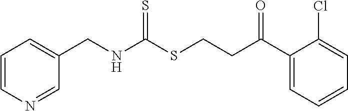 [SMILES: O=C(CCSC(=S)NCc1cccnc1)c1ccccc1Cl] | 5610/63 | |
| PMA20 | 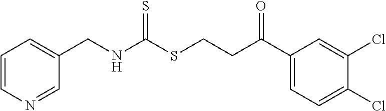 [SMILES: O=C(CCSC(=S)NCc1cccnc1)c1ccc(Cl)c(Cl)c1] | — | |
| PMA21 | 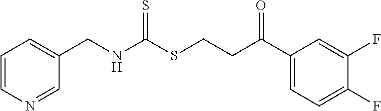 [SMILES: O=C(CCSC(=S)NCc1cccnc1)c1ccc(F)c(F)c1] | >20000/42 | |
| PMA22 | 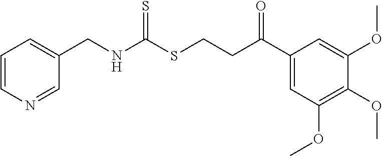 [SMILES: COc1cc(C(=O)CCSC(=S)NCc2cccnc2)cc(OC)c1OC] | 10130/131 | |
| PMA23 | 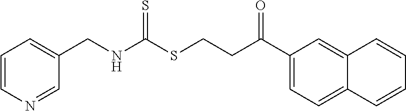 [SMILES: O=C(CCSC(=S)NCc1cccnc1)c1ccc2ccccc2c1] | — | |
| PMA24 | 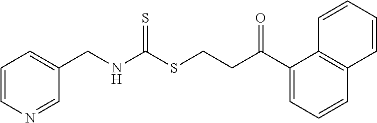 [SMILES: O=C(CCSC(=S)NCc1cccnc1)c1cccc2ccccc12] | — | |
| PMA25 | 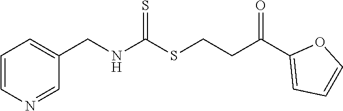 [SMILES: O=C(CCSC(=S)NCc1cccnc1)c1ccco1] | 4300/107 | |
| PMA26 | 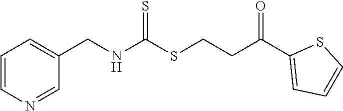 [SMILES: O=C(CCSC(=S)NCc1cccnc1)c1cccs1] | >20000/20 | |
| PMA27 | 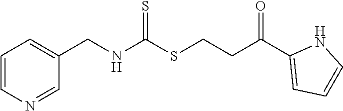 [SMILES: O=C(CCSC(=S)NCc1cccnc1)c1ccc[nH]1] | >20000/126 | |
| PMA28 | 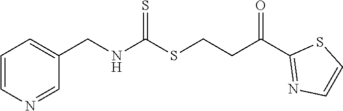 [SMILES: O=C(CCSC(=S)NCc1cccnc1)c1nccs1] | 3560/72 | |
| PMA29 | 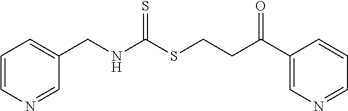 [SMILES: O=C(CCSC(=S)NCc1cccnc1)c1cccnc1] | 3700/106 | |
| PMA30 | 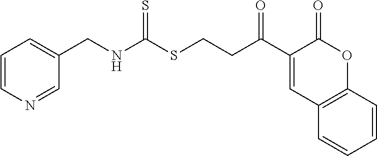 [SMILES: O=C(CCSC(=S)NCc1cccnc1)c1cc2ccccc2oc1=O] | — | |
| PMA31 | 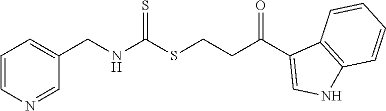 [SMILES: O=C(CCSC(=S)NCc1cccnc1)c1c[nH]c2ccccc12] | >20000/— | |
| PMA32 | 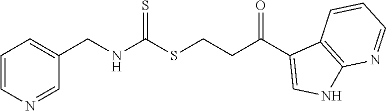 [SMILES: O=C(CCSC(=S)NCc1cccnc1)c1c[nH]c2ncccc12] | 1000/182 | |
| PMA33 | 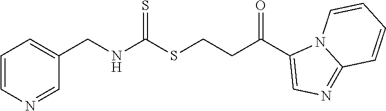 [SMILES: O=C(CCSC(=S)NCc1cccnc1)c1cnc2ccccn12] | 18150/101 | |
| PMA34 | 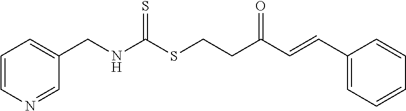 [SMILES: O=C(/C=C/c1ccccc1)CCSC(=S)NCc1cccnc1] | — | |
| PMA35 | 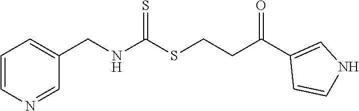 [SMILES: O=C(CCSC(=S)NCc1cccnc1)c1cc[nH]c1] | 4340/30 | |
| TPPA | 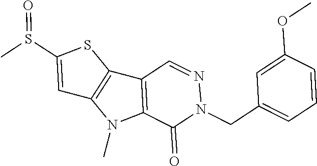 [SMILES: COc1cccc(Cn2ncc3c4sc(S(C)=O)cc4n(C)c3c2=O)c1] | — | |
| QSA | 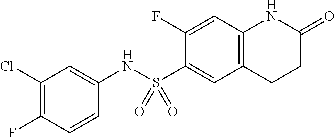 [SMILES: O=C1CCc2cc(S(=O)(=O)Nc3ccc(F)c(Cl)c3)c(F)cc2N1] | — | |
| QS | 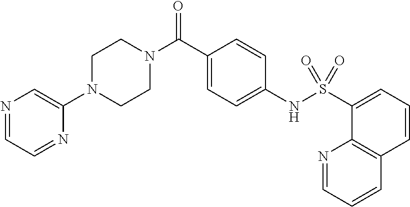 [SMILES: O=C(c1ccc(NS(=O)(=O)c2cccc3cccnc23)cc1)N1CCN(c2cnccn2)CC1] | — | |
| SAI | 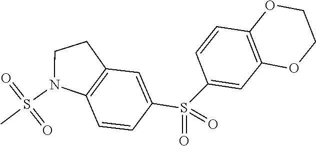 [SMILES: CS(=O)(=O)N1CCc2cc(S(=O)(=O)c3ccc4c(c3)OCCO4)ccc21] | — | |
| BIMP | 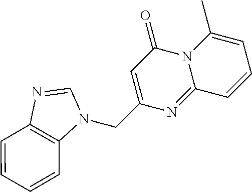 [SMILES: Cc1cccc2nc(Cn3cnc4ccccc43)cc(=O)n12] | — | |
| TPC | 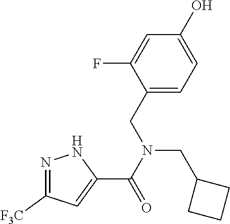 [SMILES: O=C(c1cc(C(F)(F)F)n[nH]1)N(Cc1ccc(O)cc1F)CC1CCC1] | — | |
| CA2 | 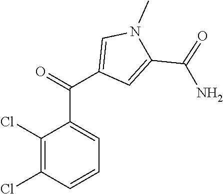 [SMILES: Cn1cc(C(=O)c2cccc(Cl)c2Cl)cc1C(N)=O] | 17000/— | |
| CA4 | 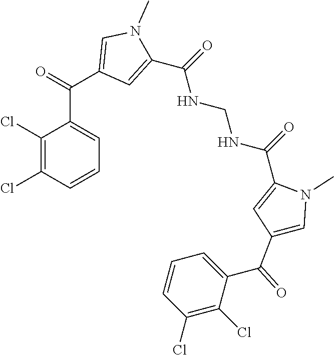 [SMILES: Cn1cc(C(=O)c2cccc(Cl)c2Cl)cc1C(=O)NCNC(=O)c1cc(C(=O)c2cccc(Cl)c2Cl)cn1C] | 3.2/— | |
| CA5 | 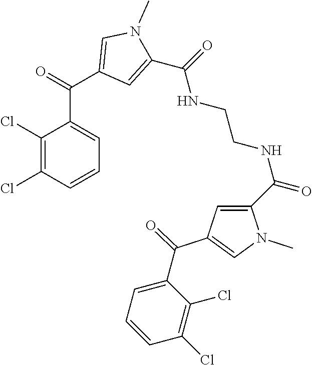 [SMILES: Cn1cc(C(=O)c2cccc(Cl)c2Cl)cc1C(=O)NCCNC(=O)c1cc(C(=O)c2cccc(Cl)c2Cl)cn1C] | 14/— | |

*Max. Resp. (%) represents the % maximum response in a biochemical assay as compared with Fructose 1,6-bisphosphate.
^{†}Indicates salt form if applicable.
TFA = trifluoroacetic acid

In certain specific embodiments, the PKM2 activator has one of the following structures:

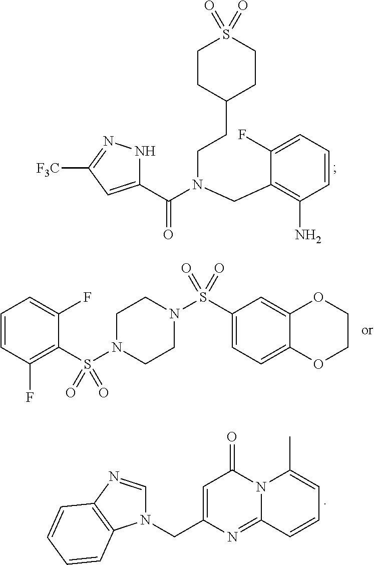

In more specific embodiments, the PKM2 activator has the following structure:0

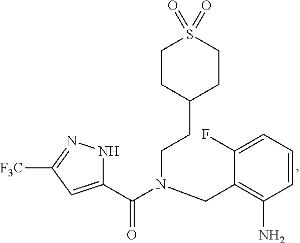 [SMILES: Nc1cccc(F)c1CN(CCC1CCS(=O)(=O)CC1)C(=O)c1cc(C(F)(F)F)n[nH]1]

or a pharmaceutically acceptable salt, tautomer or prodrug thereof.

In more specific embodiments, the PKM2 activator has the following structure:

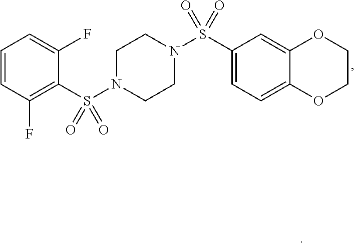 [SMILES: O=S(=O)(c1ccc2c(c1)OCCO2)N1CCN(S(=O)(=O)c2c(F)cccc2F)CC1]

or a pharmaceutically acceptable salt or prodrug thereof.

In more specific embodiments, the PKM2 activator has the following structure:

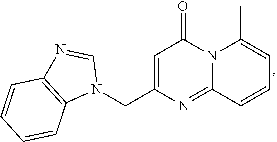 [SMILES: Cc1cccc2nc(Cn3cnc4ccccc43)cc(=O)n12]

or a pharmaceutically acceptable salt or prodrug thereof.

In certain embodiments, the PKM2 activator selectively lowers glutathione levels in cancer cells.

In some specific embodiments, a method for treatment of cancer in a patient in need thereof, the method comprising reducing glutathione levels in cancer cells of the patient and administering to the patient an anti-cancer drug having a mechanism of action that increases production of reactive oxygen species in cancer cells upon administration to the patient is provided. In more specific embodiments, reducing glutathione levels in cancer cells of the patient comprises administering a therapeutic agent to the patient, wherein the therapeutic agent, for example, a PKM2 activator, reduces glutathione levels in cancer cells of the patient.

In some of those specific embodiments, the PKM2 activator is as defined herein above. In certain embodiments, the anti-cancer drug is as defined herein below. In some embodiments, the cancer is as described in the foregoing embodiments.

It is understood that any embodiment of the PKM2 activator having structure (I), including structures (Ia), (Ib) and (Ic), as set forth above, and any of the specific substituents set forth herein (e.g., R¹-R²³) in structures (I), (Ia), (Ib) and (Ic), as set forth above, may be independently combined with other embodiments and/or substituents of structures (I), (Ia), (Ib) and (Ic) to form embodiments of the inventions not specifically set forth above. In addition, in the event that a list of substituents is listed for any particular R group in a particular embodiment and/or claim, it is understood that each individual substituent may be deleted from the particular embodiment and/or claim and that the remaining list of substituents will be considered to be within the scope of the invention. It is understood that in the present description, combinations of substituents and/or variables of the depicted formulae are permissible only if such contributions result in stable compounds.

The PKM2 activators of embodiments of the present invention can be prepared according to any number of methods known in the art, including those described in U.S. Pat. No. 9,394,257, the entirety of which is incorporated herein by reference.

Additionally, PKM2 activators in certain embodiments of the invention include the compounds disclosed in, e.g., U.S. Publication Numbers 2012/0122849, 2014/0011804, 2016/0280697, 2011/0046083, 2011/0195958, 2015/0307473 and PCT Publication Numbers WO 2014/074848, WO 2012/056319, WO 2013/005157, and WO 2012/092442 the full disclosures of which are herein incorporated by reference in their entirety for all purposes.

### II. Anti-Cancer Drugs

Embodiments of the present invention relate to the synergistic relationship between certain anti-cancer drugs and PKM2 activators, i.e., anti-cancer drugs having a mechanism of action that increases production of ROS in cancer cells. In some embodiments, such anti-cancer drugs are referred to as “ROS producing anti-cancer drugs” throughout this disclosure. Stereoisomers, pharmaceutically acceptable salts, tautomers and prodrugs of anti-cancer drugs are included within the scope of certain embodiments. Anti-cancer drug therapy regimens, such as anthracycline-based treatments, are widely used to treat multiple types of cancer, but are also known to cause significant side effects. In some embodiments a method for administering a treatment regimen that combines PKM2 activators with certain anti-cancer drugs, which advantageously increases the therapeutic index by selectively sensitizing cancer cells toward anti-cancer drugs, to a patient is provided.

Accordingly, in certain embodiments, the anti-cancer drug is selected from the group consisting of anthracyclines, anthracenediones, proteasome inhibitors, kinase inhibitors, and HSP90 inhibitors. In specific embodiments, the anti-cancer drug is an anthracycline or an anthracenedione, for example, doxorubicin, daunorubicin, mitoxantrone, epirubicin, idarubicin, nemorubicin, pixantrone, sabarubicin, or valrubicin. In some embodiments, the anti-cancer drug is an anthracycline. In other embodiments, the anti-cancer drug is an anthracenedione. In certain embodiments, the anti-cancer drug is selected from the group consisting of doxorubicin, daunorubicin, and mitoxantrone.

In certain other embodiments, the anti-cancer drug is a proteasome inhibitor, for example, bortezomib or N-benzyloxycarbonyl-Ile-Glu(O-tert-butyl)-Ala-leucinal (PSI). In certain embodiments, the anti-cancer drug is a kinase inhibitor, for example, sorafenib. In some other embodiments, the anti-cancer drug is a HSP90 inhibitor, for example, retaspimycin hydrochloride (i.e., IPI-504) or 17-allylamino-17-demethoxygeldanamycin (i.e., 17-AAG).

Additionally, anti-cancer drugs having a mechanism of action that increases production of reactive oxygen species upon administration to a patient in certain embodiments of the invention include taxanes (e.g., paclitaxel and docetaxel), vinca alkaloids (e.g., vincristine and vinblastine), anti-metabolites (e.g., anti-folates), platinum coordinating complexes (e.g., cisplatin, carboplatin and oxaliplatin), arsenic trioxide (As₂O₃), 2-methoxyestradiol, retinoid derivatives (e.g., N-(4-hydroxyphenyl) retinamide), ionizing radiation, a glutathione disulfide mimetic (e.g., NOV-002), an inhibitor of systine/glutamate transporter XCT (e.g., Sulphasalazine), an inhibitor of glucose-6-phosphate dehydrogenase (e.g., 6-anicotinamide), L-asparaginase, glutaminase inhibitors (e.g., dibenzophenanthridine), glutamate-cysteine ligase complex inhibitors (e.g., Buthionine sulphoximine), G202, COX2 inhibitors (e.g., celecoxib), nelfinavir, PARP inhibitors, erastin, lanperasone, mutant IDH1 and IDH2 isoform inhibitors (e.g., AGX-891, AG-221), camptothecin, inostamycin, Adriamycin, as well as the compounds disclosed in, e.g., U.S. Publication Numbers 2015/0197498, 2011/0053938, 2012/0259004 and PCT Publication Number WO 2012/123076, the full disclosures of which are herein incorporated by reference in their entirety for all purposes.

### III. Compositions and Administration

In other embodiments, the present invention is directed to a pharmaceutical composition comprising a pharmaceutically acceptable carrier or excipient, a PKM2 activator and an anti-cancer drug having a mechanism of action that increases production of reactive oxygen species in cancer cells upon administration to a patient in a therapeutically effective amount.

For the purposes of administration, the PKM2 activator and/or anti-cancer drug of the present invention may be administered as raw chemicals or may be formulated as pharmaceutical compositions. In certain embodiments, the components are formulated together. That is, certain pharmaceutical compositions of the present invention comprise a PKM2 activator and an anti-cancer drug and a pharmaceutically acceptable carrier, diluent or excipient. In other embodiments, the PKM2 activator and anti-cancer drug are formulated and administered separately.

The PKM2 activator and anti-cancer drug are present in their respective compositions (or the same composition) in an amount which is effective to treat a particular disease or condition of interest—that is, in an amount sufficient to treat various cancers, and preferably with acceptable toxicity to the patient. PKM2 activity of PKM2 activators can be determined by one skilled in the art, for example, as described in the Examples below. Appropriate concentrations and dosages for each respective component can be readily determined by one skilled in the art.

Administration of the PKM2 activator and anti-cancer drug, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining compounds (i.e., a PKM2 activator and/or an anti-cancer drug) with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered using certain embodiments of the methods of the invention will, in any event, contain a therapeutically effective amount of a PKM2 activator and/or a ROS producing anti-cancer drug, or pharmaceutically acceptable salts thereof, for treatment of a cancer in accordance with the teachings of embodiments of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When a pharmaceutical composition is in the form of a capsule, for example, a gelatin capsule, it may contain, in addition to materials of the above type, a liquid carrier such as polyethylene glycol or oil.

A pharmaceutical composition for used in the present method may be in the form of a liquid, for example, an elixir, syrup, solution, emulsion or suspension. The liquid may be for oral administration or for delivery by injection, as two examples. When intended for oral administration, preferred composition contain, in addition to the therapeutic compound(s), one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions used in certain embodiments of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition used in embodiments of the invention intended for either parenteral or oral administration should contain an amount of a PKM2 and/or a ROS producing anti-cancer drug such that a suitable dosage will be obtained.

A pharmaceutical composition to be use for certain embodiments of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

A pharmaceutical composition for use in some embodiments of the invention may be intended for rectal administration, in the form, for example, of a suppository, which will melt in the rectum and release the drug (e.g., PKM2 activator and/or ROS producing anti-cancer drug). The composition for rectal administration may contain an oleaginous base as a suitable nonirritating excipient. Such bases include, without limitation, lanolin, cocoa butter and polyethylene glycol.

A pharmaceutical composition for use in embodiments of the invention may include various materials, which modify the physical form of a solid or liquid dosage unit. For example, the composition may include materials that form a coating shell around the active ingredients (i.e., a PKM2 activator and/or ROS producing anti-cancer drug). The materials that form the coating shell are typically inert, and may be selected from, for example, sugar, shellac, and other enteric coating agents. Alternatively, the active ingredients may be encased in a gelatin capsule.

A pharmaceutical composition for use in certain embodiments of the invention (e.g., in solid or liquid form) may include an agent that binds to the therapeutic compound(s) and thereby assists in delivery. Suitable agents that may act in this capacity include a monoclonal or polyclonal antibody, a protein or a liposome.

A pharmaceutical composition used in certain embodiments may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of PKM2 activators and/or ROS producing anti-cancer drugs may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

A pharmaceutical composition used in certain embodiments of the invention may be prepared by methodology well known in the pharmaceutical art. For example, a pharmaceutical composition intended to be administered by injection can be prepared by combining a PKM2 activator and/or an ROS producing anti-cancer drug with sterile, distilled water so as to form a solution. A surfactant may be added to facilitate the formation of a homogeneous solution or suspension. Surfactants are compounds that non-covalently interact with the therapeutic compound(s) so as to facilitate dissolution or homogeneous suspension aqueous delivery system.

In some of the foregoing embodiments, the PKM2 activator and ROS anti-cancer drug, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Each compound used in embodiments of the invention, or pharmaceutically acceptable derivatives thereof, may also be administered simultaneously with, prior to, or after administration of one or more other therapeutic agents. Specifically, in certain embodiments, the PKM2 activator is administered the patient prior to administration of the anti-cancer drug. In more specific embodiments, the anti-cancer drug is administered within about 24 to 48 hours after administration of the PKM2 activator. In other embodiments, the anti-cancer drug is administered the patient prior to administration of the PKM2 activator.

Toxicity and therapeutic efficacy of methods described herein can be determined by standard pharmaceutical procedures in cell cultures or experimental animals, e.g., by determining the IC₅₀ and the LD₅₀ (both of which are discussed elsewhere herein) for an administered compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See, e.g., GOODMAN & GILMAN'S THE PHARMACOLOGICAL BASIS OF THERAPEUTICS, Ch. 3, 9^{th} ed., Ed. by Hardman, J., and Limbard, L., McGraw-Hill, New York City, 1996, p.46.)

Dosage amount and interval may be adjusted individually to provide plasma levels of the active species which are sufficient to maintain desired pharmacological effects. These plasma levels are referred to as minimal effective concentrations (MECs). Dosages necessary to achieve the MEC will depend on individual characteristics and route of administration. HPLC assays or bioassays can be used to determine plasma concentrations.

Dosage intervals can also be determined using MEC value. In some embodiments, methods of treatment comprise maintaining plasma levels above the MEC for 10-90% of the time, preferably between 30-90% and most preferably between 50-90%. For example, in certain embodiments, therapeutically effective amounts of PKM2 activators may range from approximately 2.5 mg/m² to 1500 mg/m² per day. Additional illustrative amounts range from 0.2-1000 mg/qid, 2-500 mg/qid, and 20-250 mg/qid.

In cases of local administration or selective uptake, the effective local concentration of the drug (i.e., the PKM2 activator and/or the anti-cancer drug) may not be related to plasma concentration, and other procedures known in the art may be employed to determine the correct dosage amount and interval.

An amount of a composition administered by certain embodiments of the method will, of course, be dependent on the subject being treated, the severity of the affliction, the manner of administration, the judgment of the prescribing physician, etc.

For some methods of treatment, compositions may, if desired, be presented in a pack or dispenser device, such as an FDA approved kit, which may contain one or more unit dosage forms containing the active ingredient. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert.

Accordingly, in certain embodiments, a kit comprising a PKM2 activator, an anti-cancer drug having a mechanism of action that increases production of reactive oxygen species in cancer cells upon administration to a patient in a therapeutically effective amount, and instructions for administering the PKM2 activator and the anti-cancer drug to a patient in need of treatment of cancer is provided. In more specific embodiments the PKM2 activator is defined in the embodiments described herein above. In some specific embodiments, the anti-cancer drug is described in the embodiments herein above. In still other embodiments, the cancer is as defined in the embodiments herein below.

### IV. Cancer Treatment

Embodiments of the methods of treatment may also find utility in a broad range of diseases and conditions, including those mediated or partially-mediated by PKM2. Such diseases may include by way of example and not limitation, cancers such as lung cancer, NSCLC (non-small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

Some embodiments of the invention include methods for treating cancers such as hematological malignancies. For example, in some embodiments the cancer is acute myeloid leukemia (AML). Other cancers include bladder cancer, or treatment of prostate cancer. Still other cancers include multiple myeloma, follicular lymphoma, acute lymphoblastic leukemia (ALL), chronic lymphocytic leukemia (CLL) and non-Hodgkin's lymphoma.

Embodiments of the inventive method can be performed in combination with administration of one or more other chemotherapeutic agents. The dosage used in some embodiments of the inventive method may be adjusted for any drug-drug reaction. In one embodiment, another chemotherapeutic agent is selected from the group consisting of mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, platinum coordination complexes (cisplatin, etc.), substituted ureas such as hydroxyurea; methylhydrazine derivatives, e.g., procarbazine; adrenocortical suppressants, e.g., mitotane, aminoglutethimide, hormone and hormone antagonists such as the adrenocorticosteriods (e.g., prednisone), progestins (e.g., hydroxyprogesterone caproate), estrogens (e.g., diethylstilbesterol), antiestrogens such as tamoxifen, androgens, e.g., testosterone propionate, and aromatase inhibitors, such as anastrozole, and AROMASIN (exemestane).

Examples of alkylating agents that can be administered in conjunction with embodiments of the present method include, without limitation, fluorouracil (5-FU) alone or in further combination with leukovorin; other pyrimidine analogs such as UFT, capecitabine, gemcitabine and cytarabine, the alkyl sulfonates, e.g., busulfan (used in the treatment of chronic granulocytic leukemia), improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil (used in the treatment of chronic lymphocytic leukemia, primary macroglobulinemia and non-Hodgkin's lymphoma), cyclophosphamide (used in the treatment of Hodgkin's disease, multiple myeloma, neuroblastoma, breast cancer, ovarian cancer, lung cancer, Wilm's tumor and rhabdomyosarcoma), estramustine, ifosfamide, novembrichin, prednimustine and uracil mustard (used in the treatment of primary thrombocytosis, non-Hodgkin's lymphoma, Hodgkin's disease and ovarian cancer); and triazines, e.g., dacarbazine (used in the treatment of soft tissue sarcoma).

Examples of antimetabolite chemotherapeutic agents that can be administered in conjunction with embodiments of the present method include, without limitation, folic acid analogs, e.g., methotrexate (used in the treatment of acute lymphocytic leukemia, choriocarcinoma, mycosis fungiodes, breast cancer, head and neck cancer and osteogenic sarcoma) and pteropterin; and the purine analogs such as mercaptopurine and thioguanine which find use in the treatment of acute granulocytic, acute lymphocytic and chronic granulocytic leukemias.

Examples of natural product-based chemotherapeutic agents that may be administered in conjunction with certain embodiments of the present method include, without limitation, the vinca alkaloids, e.g., vinblastine (used in the treatment of breast and testicular cancer), vincristine and vindesine; the epipodophyllotoxins, e.g., etoposide and teniposide, both of which are useful in the treatment of testicular cancer and Kaposi's sarcoma; the antibiotic chemotherapeutic agents, e.g., daunorubicin, doxorubicin, epirubicin, mitomycin (used to treat stomach, cervix, colon, breast, bladder and pancreatic cancer), dactinomycin, temozolomide, plicamycin, bleomycin (used in the treatment of skin, esophagus and genitourinary tract cancer); and the enzymatic chemotherapeutic agents such as L-asparaginase.

Examples of useful COX-II inhibitors include Vioxx, CELEBREX (celecoxib), valdecoxib, paracoxib, rofecoxib, and Cox 189.

Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred are those that selectively inhibit MMP-2 and/or MMP-9 relative to the other matrix-metalloproteinases (i.e., MMP-1, MMP-3, MMP-4, MMP-5, MMP-6, MMP-7, MMP-8, MMP-10, MMP-11, MMP-12, and MMP-13).

Some specific examples of MMP inhibitors are AG-3340, RO 32-3555, RS 13-0830, and compounds selected from: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methylbenzyloxy)-benzenesulfonyl]-3-hydroxy-3-methyl-piperidine-2-carboxylic acid hydroxyamide; 3-[[(4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1 ]octane-3 -carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of these compounds.

Other anti-angiogenesis agents, other COX-II inhibitors and other MMP inhibitors, can also be used in combination with certain embodiments of the present invention.

Embodiments of the method of treatment can also be combined with administration of signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, such as HERCEPTIN (Genentech, Inc., South San Francisco, Calif.). EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein.

EGFR-inhibiting agents include, but are not limited to, the monoclonal antibodies C225 and anti-EGFR 22Mab (ImClone Systems, Inc., New York, N.Y.), the compounds ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass.).

These and other EGFR-inhibiting agents can be used in embodiments of the present invention. VEGF inhibitors, for example SU-5416 and SU-6668 (Sugen Inc., South San Francisco, Calif.), can also be combined with an inventive compound. VEGF inhibitors are described in, for example, WO 01/60814 A3 (published Aug. 23, 2001), WO 99/24440 (published May 20, 1999), PCT International Application PCT/M99/00797 (filed May 3, 1999), WO 95/21613 (published Aug. 17, 1995), WO 99/61422 (published Dec. 2, 1999), U.S. Pat. No. 5,834,504 (issued Nov. 10, 1998), WO 01/60814, WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc., Kirkland, Wash.); anti-VEGF monoclonal antibody of Genentech, Inc.; and angiozyme, a synthetic ribozyme from Ribozyme (Boulder, Colo.) and Chiron (Emeryville, Calif.). These and other VEGF inhibitors can be used in the present invention as described herein. pErbB2 receptor inhibitors, such as GW-282974 (Glaxo Wellcome plc), and the monoclonal antibodies AR-209 (Aronex Pharmaceuticals Inc., The Woodlands, Tex.) and 2B-1 (Chiron), can furthermore be combined with an inventive combination, for example, those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are all hereby incorporated herein in their entireties by reference. ErbB2 receptor inhibitors useful in the present invention are also described in U.S. Pat. No. 6,284,764 (issued Sep. 4, 2001), incorporated in its entirety herein by reference. The erbB2 receptor inhibitor compounds and substance described in the aforementioned PCT applications, U.S. patents, and U.S. provisional applications, as well as other compounds and substances that inhibit the erbB2 receptor, can be used with an inventive combination, in accordance with the present invention.

In some embodiments, the inventive method of administering a combination of PKM2 activator and anti-cancer drug can be used with the administration of other agents useful in treating cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors.

Embodiments of the inventive methods described herein can also be carried out in combination with radiation therapy, wherein the amount of PKM2 activator and anti-cancer drug is dosed in combination with the radiation therapy such that it is effective in treating the above diseases. Techniques for administering radiation therapy are known in the art, and these techniques can be used in the combination therapy described herein.

The following examples are provided for purposes of illustration, not limitation.

### EXAMPLES

### Example 1

### Synergy Between Anthracycline or Anthracenedione Compounds and PKM2 Activator

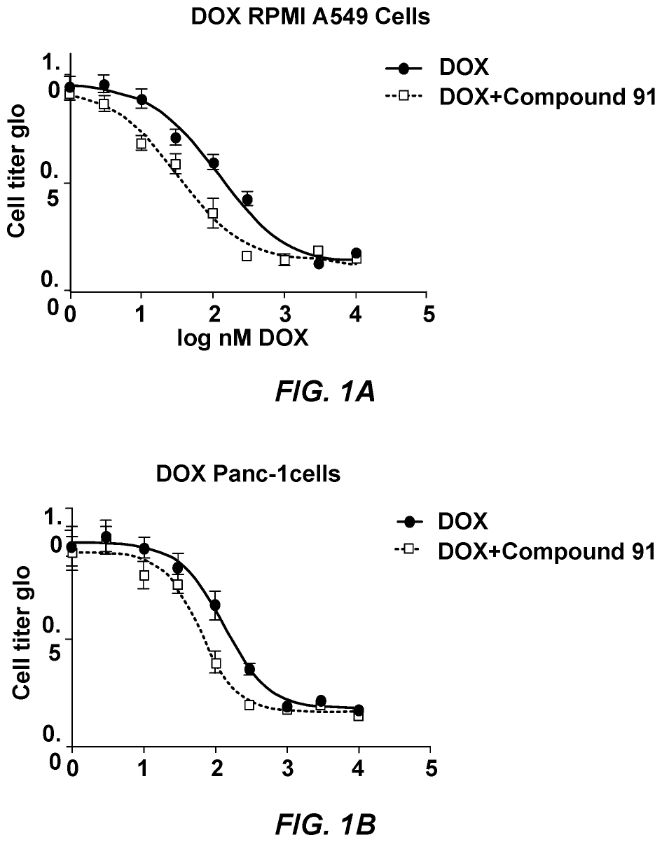

A549 and Pancl cells were plated using 384-well plates at a density of 1,200 cells per well. Cells were plated in RPMI media and allowed to adhere for 24 hours at 37° C. Cells were then treated with concentration gradients of doxorubicin (FIGS. 1A-B), daunorubicin (FIG. 1C) or mitoxantrone (FIG. 1D) in the presence and absence of Compound 91 at a concentration of 4 μM in replicates of 7 per condition. Cells were also treated with Compound 91 alone as a control. Cell viability was determined according to the Cell-titer-Glo assay kit and protocol from Promega Biosciences, LLC (Madison, Wis.).

The data clearly show increased activity when Compound 91 was used in combination with each of the anthracycline-based or anthracenedione-based anti-cancer drugs. Specifically, A549 and Pancl cells were tested for cell variability, which showed increased activity relative to each compound tested alone (FIGS. 1A-F).

### Example 2

### Synergy between HSP90 Inhibitor and PKM2 Activator

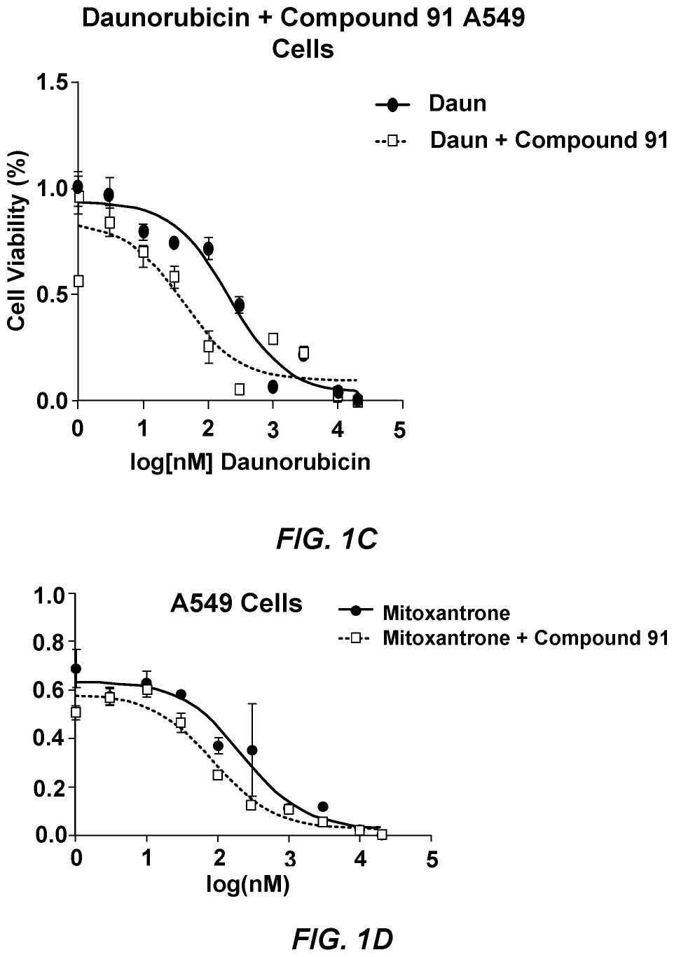

A549 cells were plated according to the procedure described in Example 1. Cells were treated using a concentration gradient of IPI-504 in the presence and absence of Compound 91 at a concentration of 4 μM in replicates of 7 per condition. Cell was determined using the Cell-titer-Glo assay kit according to the procedure in Example 1 (results shown in FIG. 2). The resultant decrease in EC₅₀ concentration from 59.94 nM for IPI-504 alone to 36.45 nM for IPI-504 in combination with Compound 91 shows a synergistic relationship between PKM2 activators and HSP90 inhibitors.

### Example 3

### Synerrgy between Kinase Inhibitor and PKM2 Adtivator

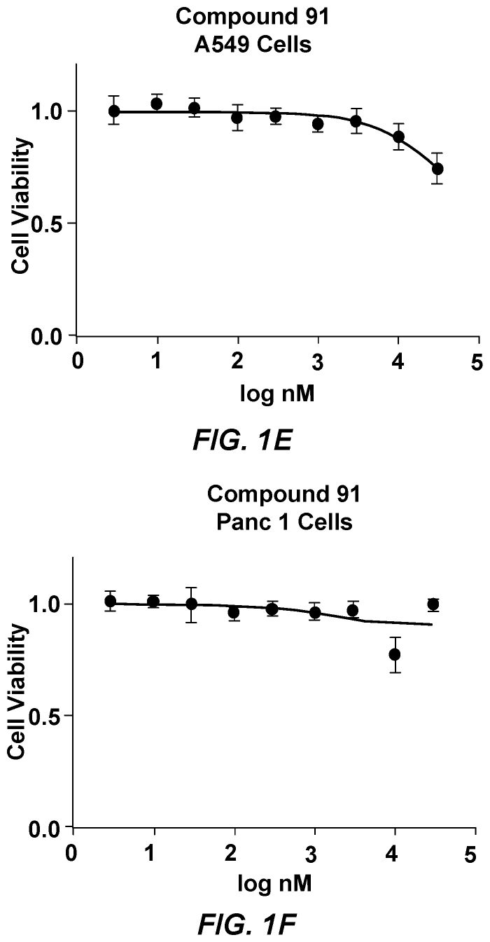

A549 cells were plated according to the procedure described in Example 1. Cells were treated using a concentration gradient of sorafaib in the presence and absence Compound 91 at a concentration of 4 μM in replicates of 7. Cell viability was determined using the Cell-titer-Glo assay kit according to the procedure described in the examples above, and the resultant data (FIG. 3) show a greater than 4-fold decrease in EC₅₀ (i.e., from 30,126 nM to 7,126 nM) when Compound 91 and sorafenib are used in combination.

### Example 4

### Synergy between Proteasome Inhibitor and PKM2 Activator

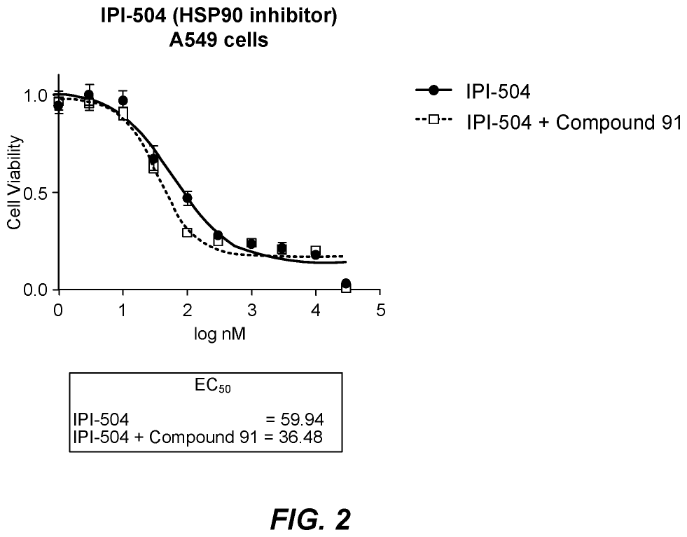

A549 cells were plated according to the procedure described in Example 1. Cells were treated using a concentration gradient of bortezomib in the presence and absence Compound 91 at a concentration of 4 μM in replicates of 7 per condition. Cell viability was determined using the Cell-titer-Glo assay kit according to the examples above, and the resultant data (FIG. 4A) show an almost 2-fold decrease in EC₅₀ concentration (i.e., from 43.36 nM to 27.04 nM) when Compound 91 and bortezomib are used in combination.

Additionally, samples were treated with bortezomib in the presence and absence of DASA, PP8 and Compound 91 at a concentration of 10 μM in replicates of 10 per condition. Cell viability was determined using the Cell-titer-Glo assay kit as described in the examples above. The resultant data show a decrease in EC₅₀ when each of the representative PKM2 activators are used in combination with bortesamib (FIG. 4B). Thus, each of the representative PKM2 activators improves activity, with some compounds resulting in a decrease of EC₅₀ concentration of almost 2-fold.

### Example 5

### Lack of Syneegy between Non-Ros Producing Drugs and PKM2 Activators

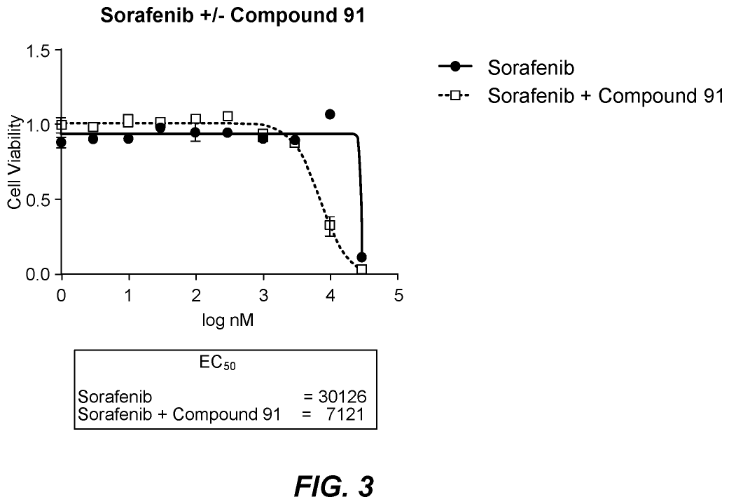

A549 and Panc1 cells were plated according to the procedure described in Example 1. Cells were treated using a concentration gradient of rapamycin (FIGS. 5A-B) or Vosaroxin (a TOPOII poison; FIG. 5C), both in the presence and absence of Compound 91 at a concentration of 4 μM in replicates of 7 per condition. Cell viability was determined using the Cell-titer-Glo assay kit according to the procedure described in the examples above. The data show no significant change for treatments in the presence and absence of Compound 91.

Without wishing to be bound by theory, the lack of synergy when PKM2 activators are combined with rapamycin and Vosaroxin elucidates the mechanism of action for such a synergistic effect. That is, when the concentration reactive oxygen species is not increased by the administration of a compound (i.e., rapamycin and Vosaroxin), combination of those compounds with PKM2 activators results in no synergistic increase in activity (FIGS. 5A-C).

### Example 6

### Metabolic Effect of PKM2 Activator

A549 cells were treated with either DMSO (blank control) or 30 Compound 91 in replicates of 6 in serum free media. After treatment for 24 hours, cells were harvested and analyzed by the University of Utah Metabolomics core to determine alterations in metabolites caused by treatment with Compound 91. The results are summarized in Table 2 below.

TABLE 2
Metabolic Effects of PKM2 Activator Treatment

| | Fold Change | | |
|---|---|---|---|
| Metabolite | (D/T) | p-value | −log (p) |
| Glutathione | 10.9 | 1.63 × 10⁻⁶ | 5.7867 |
| Threonine | 0.53667 | 9.50 × 10⁻⁶ | 5.0221 |
| rhamnose (or isomer) | 5.6188 | 1.82 × 10⁻⁵ | 4.7407 |
| 4-hydroxyproline | 0.46357 | 2.59 × 10⁻⁵ | 4.5871 |
| Glutamine | 2.2019 | 2.75 × 10⁻⁵ | 4.5602 |
| Cysteine | 2.55 | 4.64 × 10⁻⁵ | 4.3331 |
| 3-phosphoglycerate | 2.9719 | 7.97 × 10⁻⁵ | 4.0985 |
| N-acetylaspartate | 0.56599 | 1.1063 × 10⁻⁴ | 3.9561 |
| Asparagine | 0.45322 | 2.2472 × 10⁻⁴ | 3.6483 |

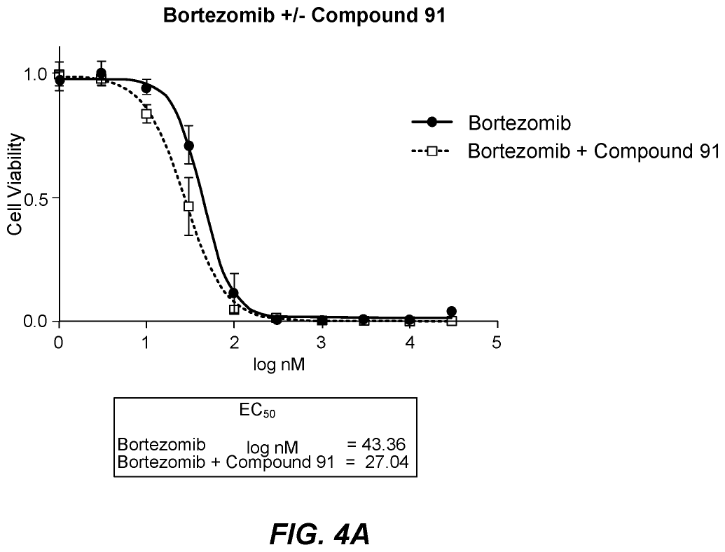

In addition to the data provided in Table 2, A549 cells were prepared according to Example 1 above. Those cells were treated with representative PKM2 activators DASA, PP8 and Compound 9l at concentrations at the concentrations indicated in FIG. 6A, and in replicates of 10 for each condition. Cells were treated for 48 hours, and then glutathione levels were determined using a GSH-Glo assay kit and procedure from Promega. FIG. 6A shows decreased levels of glutathione for each PKM2 activator, even at concentrations as low as 0.1 μM. Additionally, FIG. 6B shows treatment as described above for 24 hours with indicated changes shown relative to a blank control (DMSO) as determined by the GSH-Glo assay.

Without wishing to be bound by theory, it is thought that the metabolic effect of treatment using PKM2 activators effectively combines with the mechanism of action for certain drugs that increase production of ROS in cancer cells, thus resulting in a highly efficacious treatment for cancers. In brief, PKM2 activators lower glutathione levels in cells, which work to synergistically combine with anti-cancer drugs that increase production of reactive oxygen species in cancer cells as a mechanism of action.

### Example 7

### In Vivo Synerby between Anthracycline Compounds and PKM2 Activator

A xenograph study was performed using A549 lung cancer cells to interrogate the in vivo synergy between a representative ROS-producing anti-cancer drug and a PKM2 activator. Mice (6-8 week old female athymic nude) were housed under standard conditions, and allowed food and water ad libitum and injected with 1×10⁷ A549 cells per mouse. Upon reaching a tumor volume of approximately 100-200 mm³ mice were treated with a control (vehicle alone), doxorubicin, Compound 91 or a combination of doxorubicin and Compound 91. The doxorubicin was administered orally at a concentration of 2 mg/kg every 2 days. Compound 91 was administered orally at a concentration of 200 mg/kg every day.

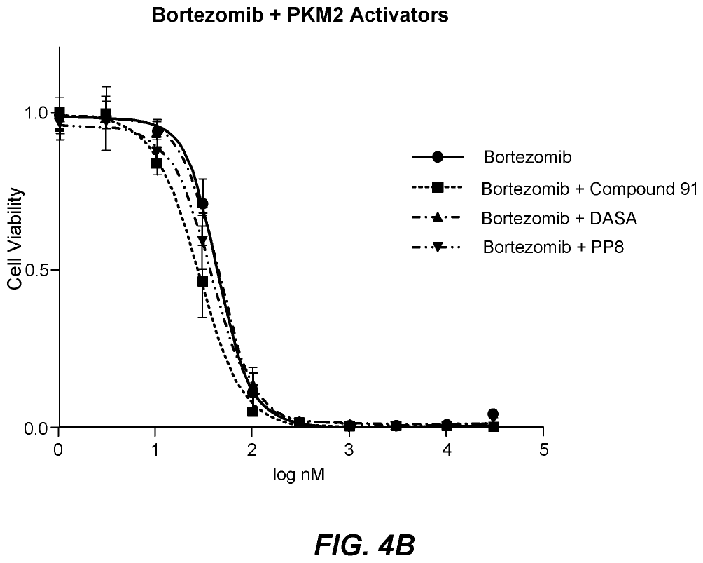

Tumor volume and body weights were measured and recorded twice weekly (FIGS. 7A and 7B, respectively). Upon completion of the study, mice were euthanized and tumor tissues were harvested. As the data of FIG. 7A shows, treatment with the combination of doxorubicin and Compound 91 showed the best reduction of tumor volume. In addition, the data of FIG. 7B show no significant body weight reduction or fluctuation while the combination of drugs was being administered.

### Example 8

### In Vivo Synergy between Anthracycline Compounds and PKM2 Activator

### 1. Summary

The therapeutic efficacy of Compound 91 alone and in combination with Doxorubicin in the treatment of subcutaneous A549 human lung cancer model was evaluated.

A549 human lung cancer cells were inoculated in BALB/c nude mice and treatment was initiated when tumors reached a mean volume of approximately 100 mm³. In this study, only Group 6, treated with Compound 91 at 200 mg/kg and Doxorubicin at 2 mg/kg, demonstrated significant anti-tumor efficacy (TGI: 29%, p<0.05) compared to vehicle control group. Although no statistically significant inhibition of tumor growth was observed, treatment with Compound 91 (100 mg/kg) or Doxorubicin (2 mg/kg) alone and in combination resulted in a decrease in the mean tumor volume. For Group 4, treated with Compound 91 at 200 mg/kg and saline, there were 9 tumors (TV<970 mm³) with mean tumor volume of 754.25 mm³ and 1 tumor (2526.23 mm³) at termination. No statistically significant decrease in tumor weight was observed for all treatment groups compared to vehicle control group.

### 2. Animal Welfare and Regulatory Compliance Statement

All studies were conducted following an approved IACUC protocol. Although this study was not conducted in accordance with the FDA Good Laboratory Practice regulations, 21 CFR Part 58, all experimental data management and reporting procedures were in strict accordance with applicable Guidelines and Standard Operating Procedures.

### 3. Quality Control

All study procedures were conducted by qualified personnel, and were in accordance with the approved IACUC protocol and Standard Operating Procedures. All the data reported was reviewed by the Study Director.

### 4. Materials and Methods

4.1 Animals

TABLE 3

| Parameter | Female |
|---|---|
| Species | Mus musculus |
| Strain | BALB/c nude |
| Supplier | Shanghai Lingchang |
| | Biotechnology Co., |
| | Ltd (Shanghai, China) |
| No. of Animals Received | 84 (60 plus 40% excess) |
| Age of Animals at Study | 7 to 8 weeks |
| Initiation | |
| Body weight of Animals | 17.6-23.9 grams |
| at Study Initiation | |
| Animal Certificate No.: | 201801142 |

4.2 Test and Control Articles

TABLE 4

| | Lot No./ | | | | | |
|---|---|---|---|---|---|---|
| | Batch | | Physical | Conc./ | Volume/ | Storage |
| Name | No. | Supplier | description | Purity | Mass | Information |
| Compound 91 | SY1700 | Tolero | Powder | NA | 4500 mg | −20° C. |
| | 14 14-28 | Pharmaceuticals | | | | |
| Doxorubicin | NA | Tolero | Powder | NA | 27.96 mg | −20° C. |
| | | Pharmaceuticals | | | | |

4.3 Drug Formulation

TABLE 5

| | | Test Article | | | | |
|---|---|---|---|---|---|---|
| | | Dose (Free | | Physical | | |
| Drug | Conc. (mg/mL) | Base))mg/kg) | Formulation Details | Description | Storage | |
| Vehicle for | — | — | Prepared mixture of | Solution | 4° | C. |
| Compound | | | Tween-80, Ethanol, | | | |
| | | | PEG400, water at a | | | |
| | | | ratio of (2:10:30:58) | | | |
| | | | Frequency: prepared | | | |
| | | | weekly | | | |
| Compound | 10 | 100 | Added 420 mg of Compound | Solution | 4° | C. |
| 91 | | | 91 to 42 mL of vehicle, | | | |
| | | | vortexed and sonicated | | | |
| | | | for a clear solution. | | | |
| | | | Prepared once a week. | | | |
| Compound | 20 | 200 | Added 840 mg of Compound | Solution | 4° | C. |
| 91 | | | 91 to 42 mL of vehicle, | | | |
| | | | vortexed and sonicated | | | |
| | | | for a clear solution. | | | |
| | | | Prepared once a week. | | | |
| Doxorubicin | 0.2 | 2 | Dissolved 1.5 mg of | Solution | 4° | C., |

| | |
|---|---|
| Doxorubicin with 7.5 ml | protected |
| sterile saline (0.9% | from light |
| NaCl) to obtain 7.5 ml of | |
| 0.2 mg/ml dosing solution. | |

4.4 Treatment Groups

TABLE 6

| | | | | Dose | | Dose | Actual Dose |
|---|---|---|---|---|---|---|---|
| Group | | Treatment | Dose | Vol. | Dose | Frequency | Frequency & |
| No. | Mice | Description | (mg/kg) | (ml/kg) | Route | & Duration | Duration |
| 1 | 10 | Vehicle | 0 | 10 | p.o. | QD × 3 weeks | QD × 3 weeks |
| | | saline | 0 | 10 | i.v. | Q2D × 3 weeks | Q2D × 3 |
| | | | | | | | weeks |
| 2 | 10 | Doxorubicin | 2 | 10 | i.v. | Q2D × 3 weeks | Q2D × 3 |
| | | | | | | | weeks |
| | | Vehicle | 0 | 10 | p.o. | QD × 3 weeks | QD × 3 weeks |
| 3 | 10 | Compound 91 | 100 | 10 | p.o. | QD × 3 weeks | QD × 3 weeks |
| | | saline | 0 | 10 | i.v. | Q2D × 3 weeks | Q2D × 3 |
| | | | | | | | weeks |
| 4 | 10 | Compound 91 | 200 | 10 | p.o. | QD × 3 weeks | QD × 3 weeks |
| | | saline | 0 | 10 | i.v. | Q2D× 3 weeks | Q2D × 3 |
| | | | | | | | weeks |
| 5 | 10 | Compound 91 | 100 | 10 | p.o. | QD × 3 weeks | QD × 3 weeks |
| | | Doxorubicin | 2 | 10 | i.v. | Q2D × 3 weeks | Q2D × 3 |
| | | | | | | | weeks |
| 6 | 10 | Compound 91 | 200 | 10 | p.o. | QD × 3 weeks | QD × 3 weeks |
| | | Doxorubicin | 2 | 10 | i.v. | Q2D × 3 weeks | Q2D × 3 |
| | | | | | | | weeks |

4.5 Randomization 60 mice were enrolled in the study. All animals were randomly allocated to the 6 different study groups. The mean tumor size at randomization was approximately 100 mm³. Randomization was performed based on “Matched distribution” randomization method using multi-task method (StudyDirector™ software, version 3.1.399.19) on day 13.

4.6 Observations and Data Collection

After tumor cell inoculation, the animals were checked daily for morbidity and mortality. At the time of routine monitoring, the animals were checked for any effects of tumor growth and treatments on behavior such as mobility, food and water consumption, body weight gain/loss (body weight was measured twice per week, or based on request after randomization), and any other abnormalities. Mortality and observed clinical signs were recorded for individual animals.

Tumor volumes were measured twice weekly in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L).

Tumor weight was measured at the end of study based on the protocol. Dosing as well as tumor and body weight measurement were conducted in a Laminar Flow Cabinet.

4.7 Termination
The study was terminated 1 hour post the final dose on day 33.
Below are the standards for humane endpoints.
1) Severe dehydration;
2) Impaired mobility (not able to eat or drink);
3) Astasia, continuous prone or lateral position;
4) Hypopraxia, signs of muscular atrophy;
5) Labored respiration;
6) Progressive hypothermia;
7) Paralytic gait, clonic convulsions, tonic convulsions;
8) Persistent bleeding from body openings;
9) Unable to move normally due to enlarged tumor mass;
10) Unable to move normally due to significant ascites and enlarged abdomen;
11) Mouse with tumor ulceration of approximately 25% or greater on the surface of the tumor.

4.8 Statistical Analysis

Statistical analysis of differences in mean tumor volume among the groups was conducted by Independent-Samples T Test using the data collected.

All data were analyzed with SPSS (Statistical Product and Service Solutions) version 18.0 (IBM, Armonk, N.Y., U.S.). P-values were rounded to three decimal places, with the exception that raw P-values less than 0.001 were stated as P<0.001. All tests were two-sided. P<0.05 was considered to be statistically significant.

### 5. Results

5.1 Tumor Volume

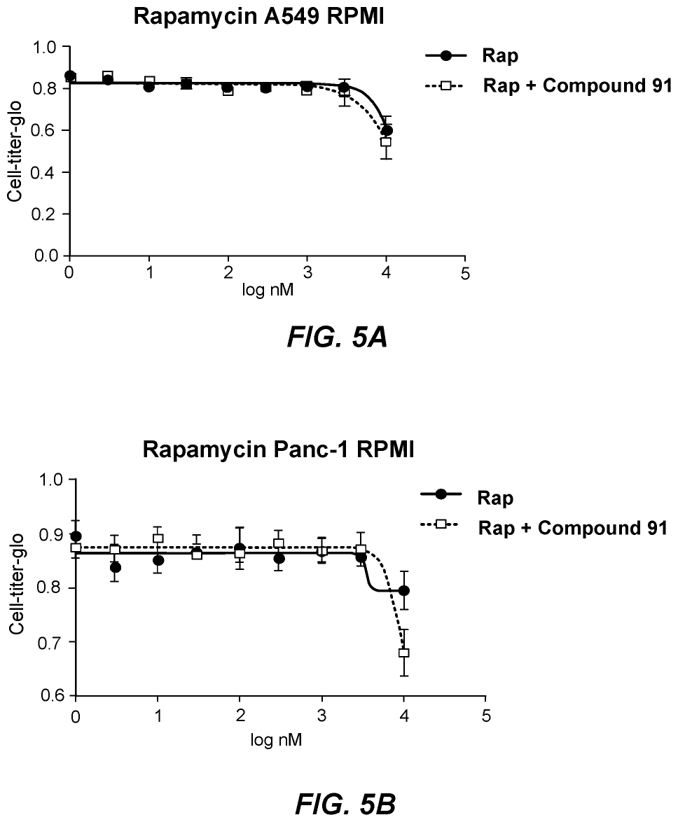

The tumor growth curves (mean tumor volume over time) of different groups are shown in FIG. 8.

5.2 Tumor Growth Inhibition

The tumor growth inhibition is summarized in Table 7 below.

TABLE 7
Antitumor Activity of Test Compound in the
Treatment of Subcutaneous A549 Human Lung
Cancer Xenograft Model in BALB/c Nude Mice

| | | Tumor Size | | | |
|---|---|---|---|---|---|
| | Treatment | (mm³)a | TGI | T/C | P |
| Group | Description | on day 33 | (%) | (%) | valueb |
| 1 | Vehicle(0 mg/kg) | 777.38 ± 62.07 | — | — | — |
| | Saline(0 mg/kg) | | | | |
| 2 | Doxorubicin | 723.15 ± 129.91 | 7 | 93 | 0.711 |
| | (2 mg/kg) | | | | |
| | Vehicle (0 mg/kg) | | | | |
| 3 | Compound | 683.54 ± 75.00 | 12 | 88 | 0.348 |
| | 91 (100 mg/kg) | | | | |
| | saline (0 mg/kg) | | | | |
| 4 | Compound | 931.45 ± 192.47 | −20 | 120 | 0.456 |
| | 91 (200 mg/kg) | | | | |
| | saline (0 mg/kg) | | | | |
| 5 | Compound | 668.99 ± 95.71 | 14 | 86 | 0.355 |
| | 91 (100 mg/kg) | | | | |
| | Doxorubicin | | | | |
| | (2 mg/kg) | | | | |
| 6 | Compound | 550.27 ± 70.27 | 29 | 71 | 0.026 |
| | 91 (200 mg/kg) | | | | |
| | Doxorubicin | | | | |
| | (2 mg/kg) | | | | |

1, aMean ± SEM; bvs. vehicle control.
2, group-2 vs. group-5, p = 0.741; group-2 vs. group-6, p = 0.257; group-3 vs. group-5, p = 0.906; group-4 vs. group-6, p = 0.079.

The tumor weight analysis is summarized in Table 8 below.

TABLE 8
Tumor Weight Analysis of Test Compound in the
Treatment of Subcutaneous A549 Human Lung
Cancer Xenograft Model in BALB/c Nude Mice

| | Treatment | Tumor Weight | TGI | T/C | P |
|---|---|---|---|---|---|
| Group | Description | (mg)a on day 33 | (%) | (%) | valueb |
| 1 | Vehicle(0 mg/kg) | 742.4 ± 57.4 | — | — | — |
| | Saline(0 mg/kg) | | | | |
| 2 | Doxorubicin | 676.9 ± 107.8 | 9 | 91 | 0.627 |
| | (2 mg/kg) | | | | |
| | Vehicle (0 mg/kg) | | | | |
| 3 | Compound 91 | 679.2 ± 72.9 | 9 | 91 | 0.531 |
| | (100 mg/kg) | | | | |
| | saline (0 mg/kg) | | | | |
| 4 | Compound 91 | 954.6 ± 155.3 | −29 | 129 | 0.256 |
| | (200 mg/kg) | | | | |
| | saline (0 mg/kg) | | | | |
| 5 | Compound 91 | 640.5 ± 86.1 | 14 | 86 | 0.374 |
| | (100 mg/kg) | | | | |
| | Doxorubicin | | | | |
| | (2 mg/kg) | | | | |
| 6 | Compound 91 | 582.9 ± 75.4 | 21 | 79 | 0.132 |
| | (200 mg/kg) | | | | |
| | Doxorubicin | | | | |
| | (2 mg/kg) | | | | |

1, aMean ± SEM; bvs. vehicle control.
2, group-2 vs. group-5, p = 0.814; group-2 vs. group-6, p = 0.526; group-3 vs. group-5, p = 0.760; group-4 vs. group-6, p = 0.067.

5.3 Body Weight

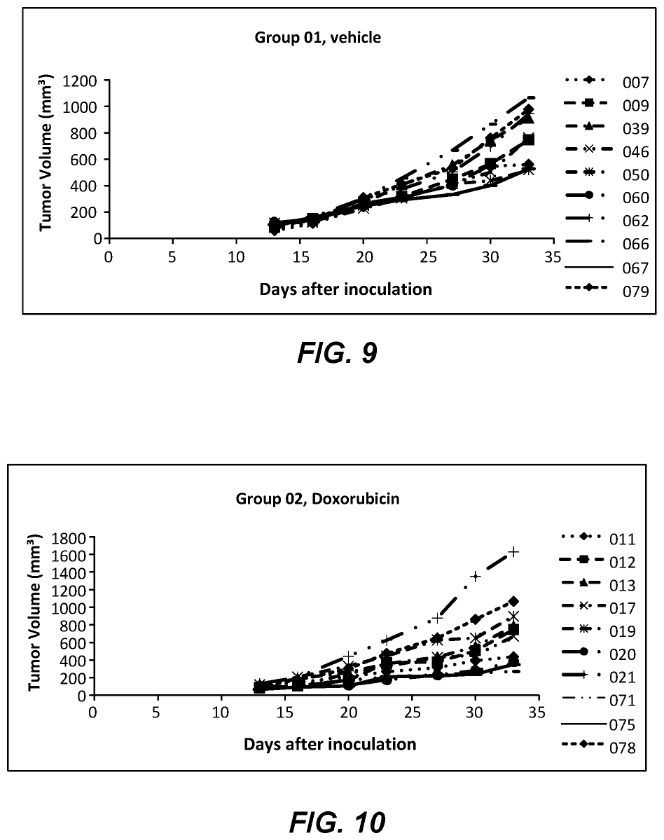

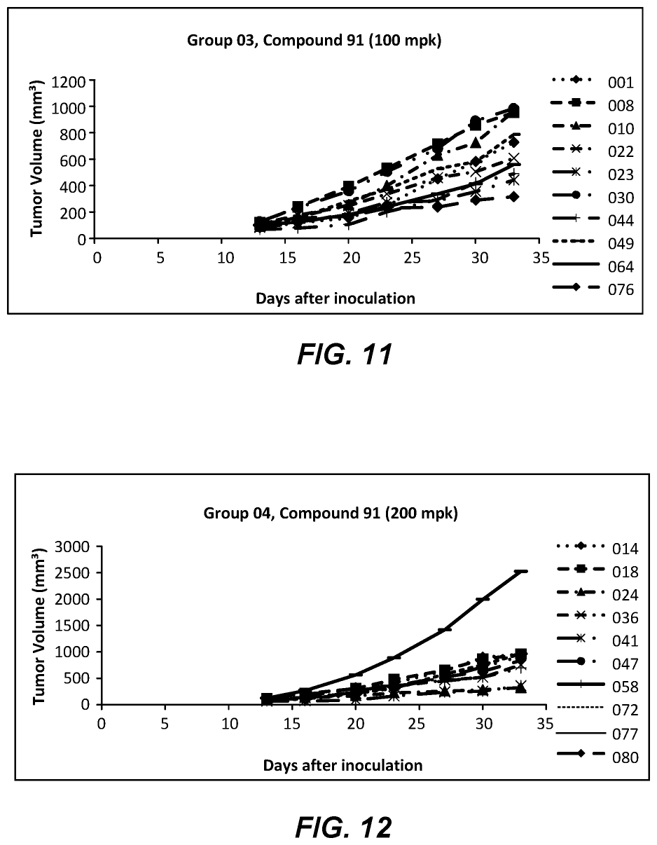

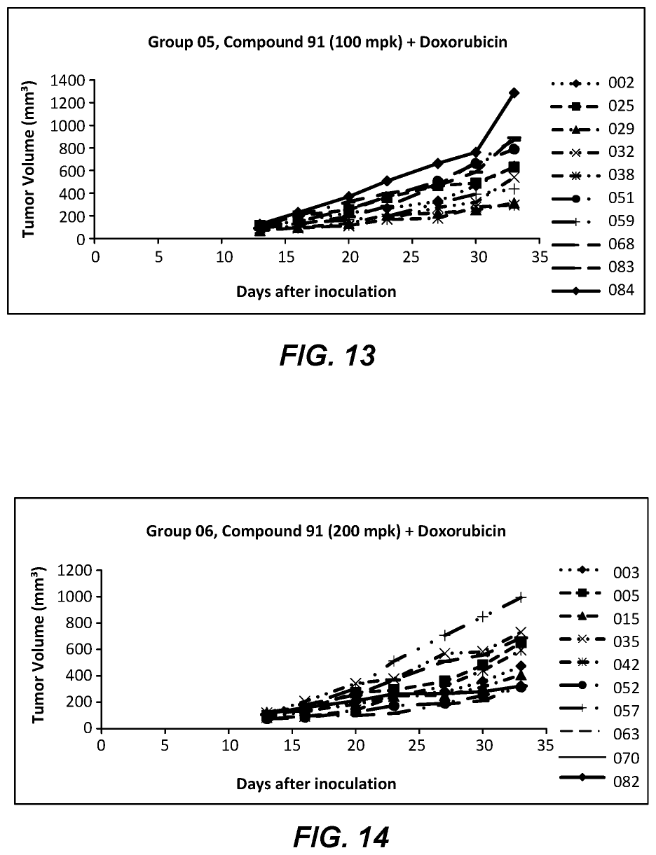

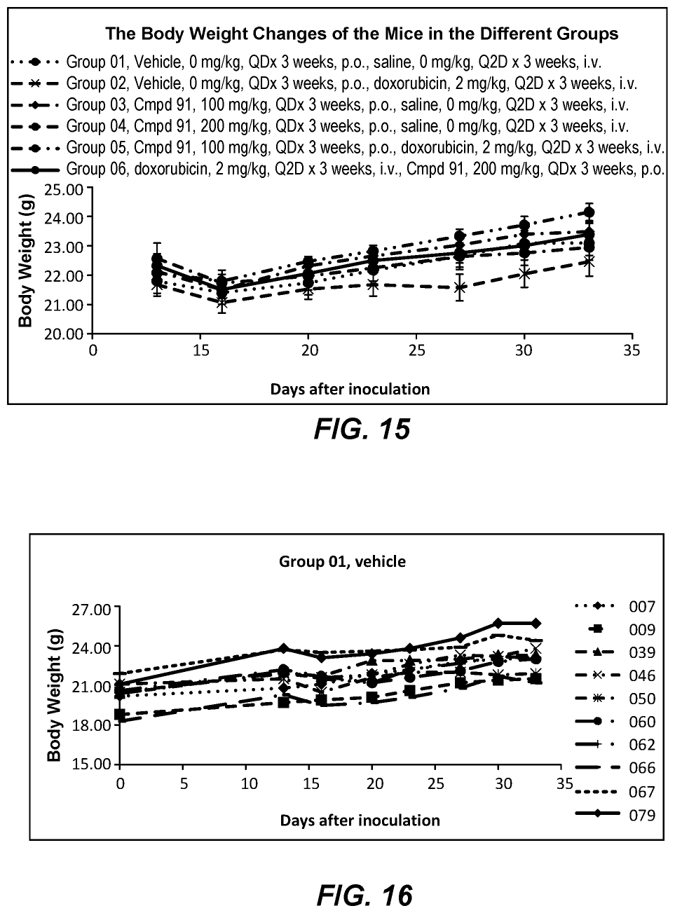

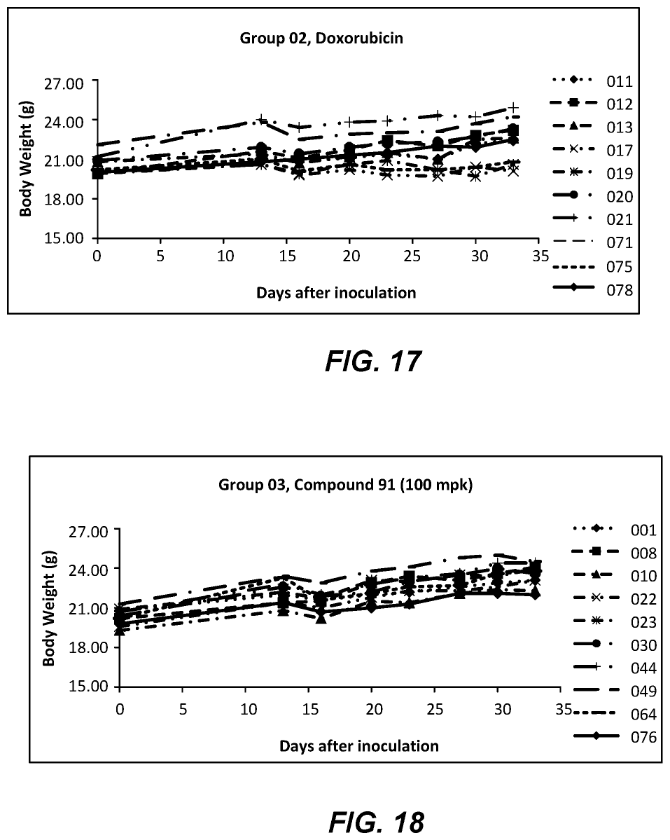

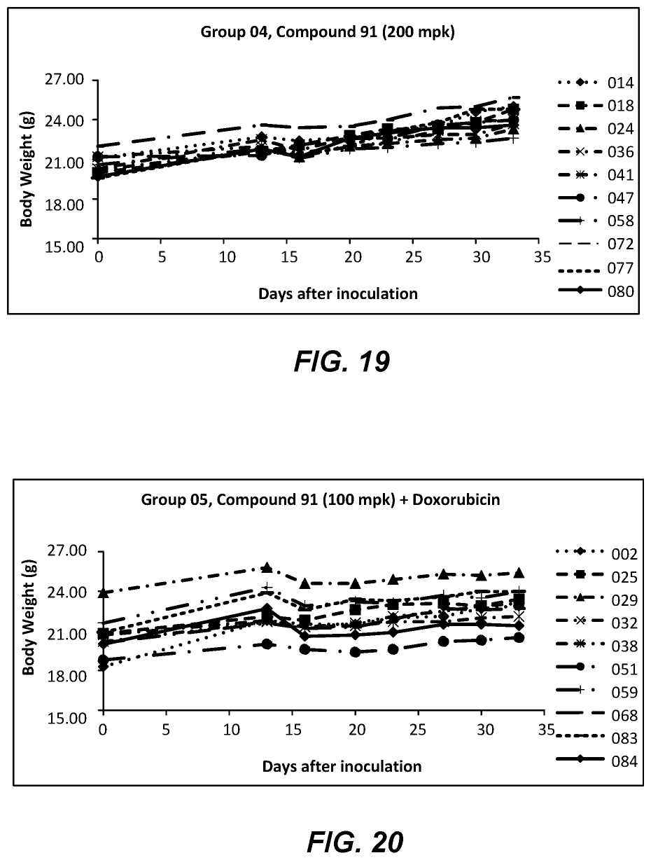

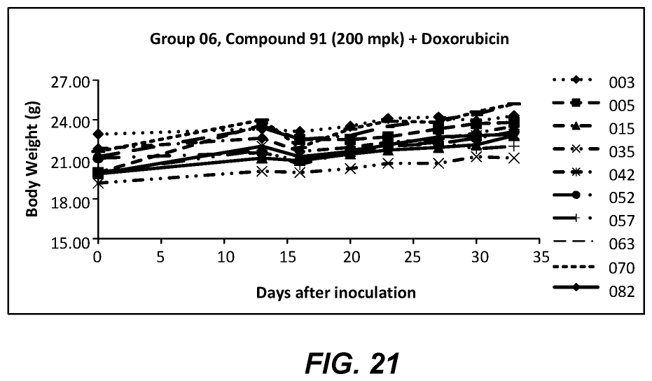

The results of mean body weight changes in the tumor bearing mice are shown in FIG. 15. The results of individual body weight changes in the tumor bearing mice are shown in FIGS. 16-21.

5.4 Mortality and Tolerability

No adverse effect on body weight was found in all six groups.

5.5 Biospecimen Collection and Disposition

Tumor samples were collected from all groups lhr post the final dosing, processed and stored at predefined conditions per requirements from the study protocol.

### 6. Result Summary

In this study, the therapeutic efficacy of Compound 91 alone and in combination with Doxorubicin in the treatment of subcutaneous A549 human lung cancer model in BALB/c nude mice was evaluated.

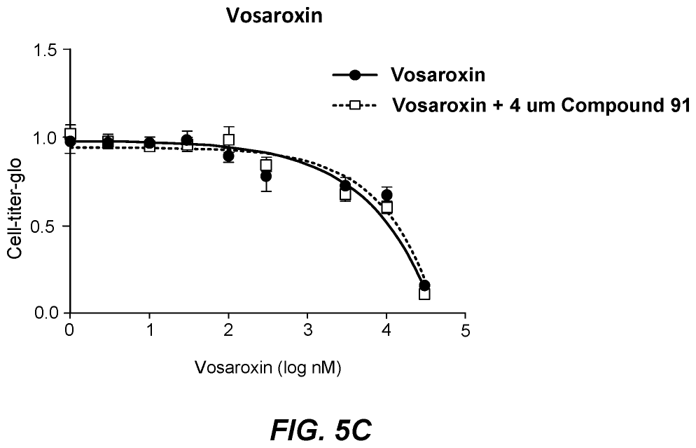

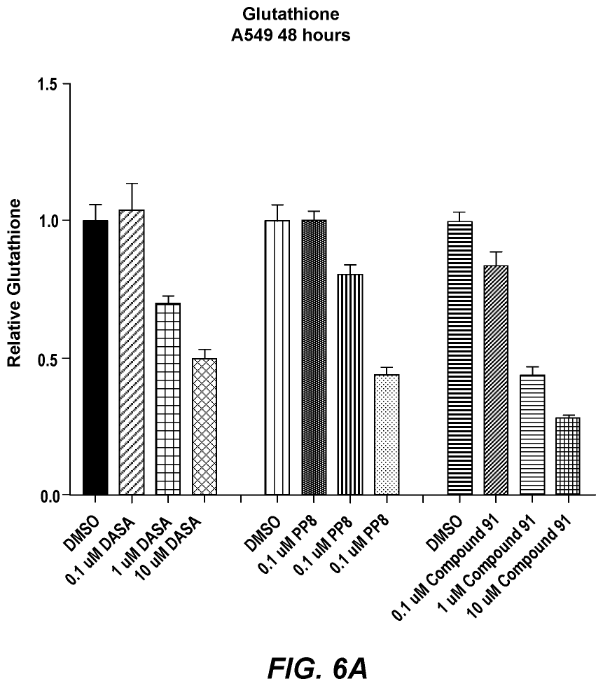

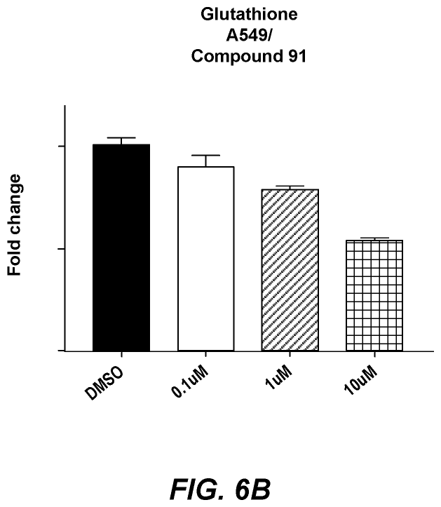

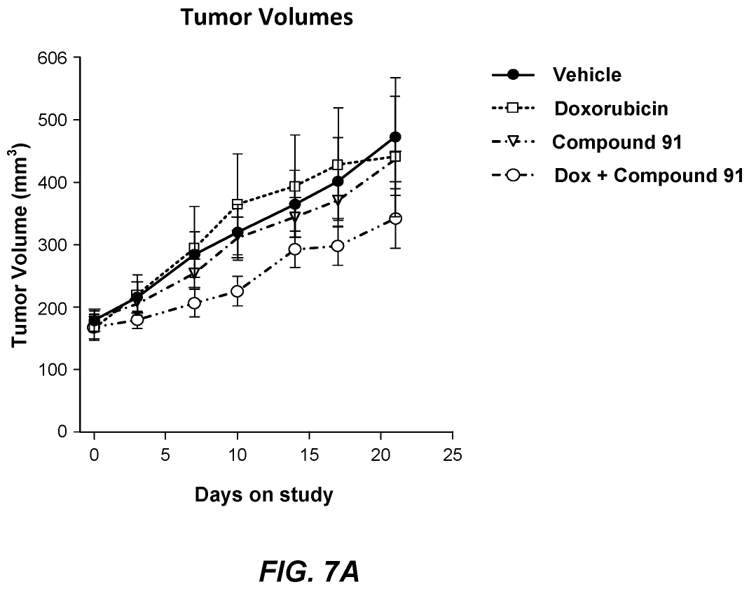

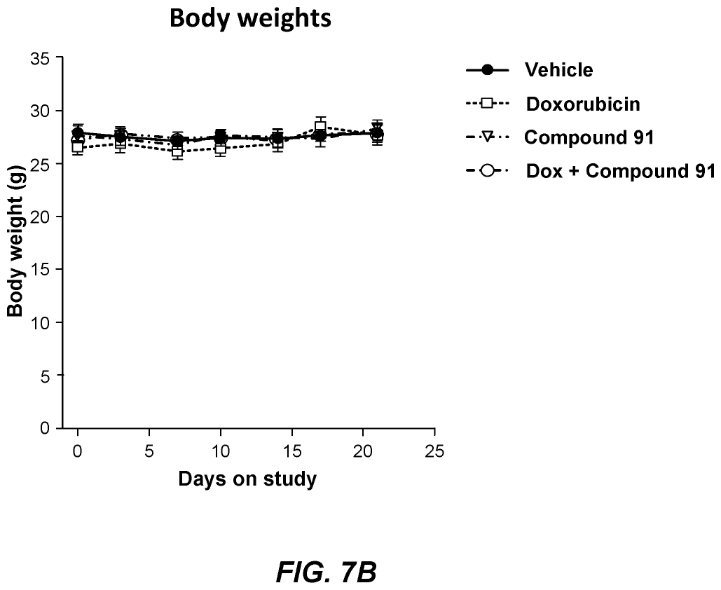

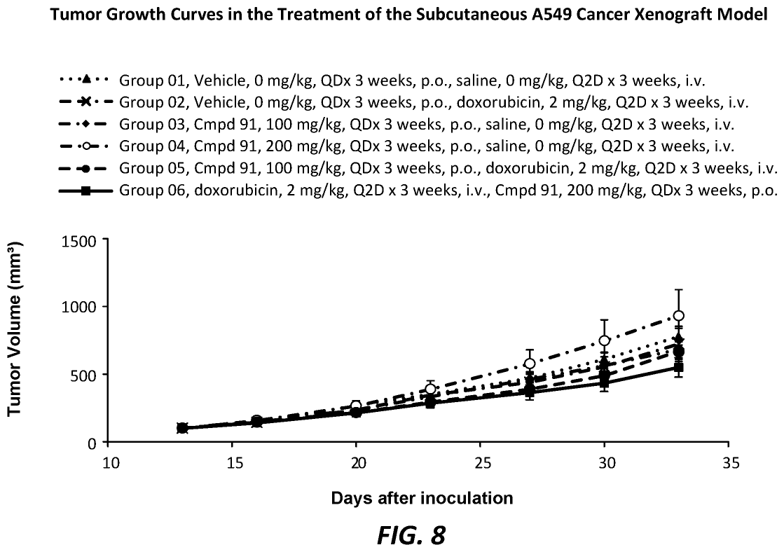

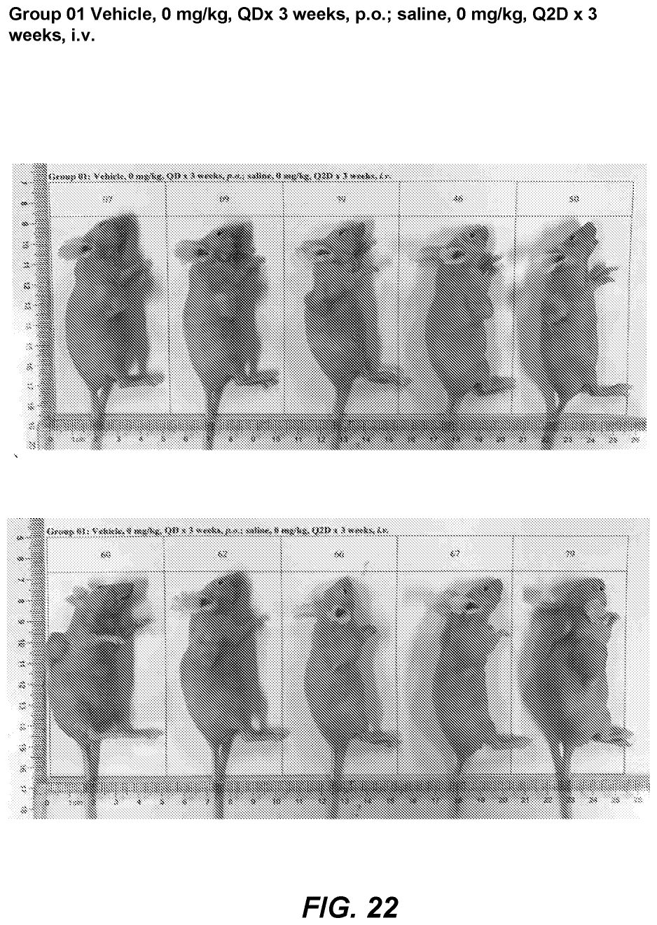

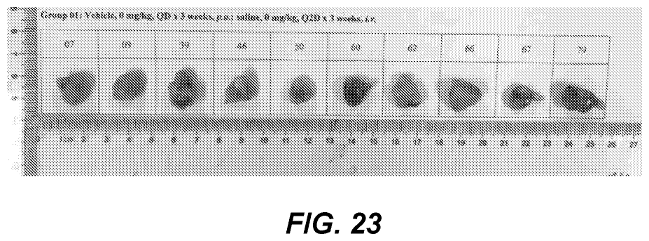

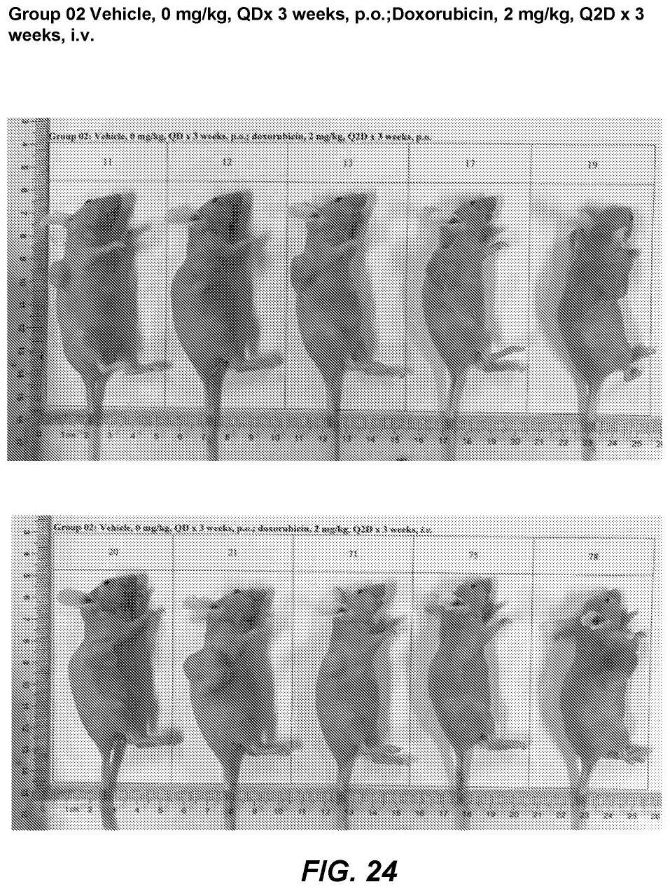

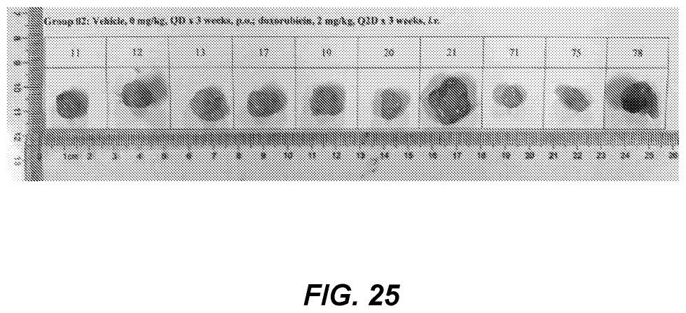

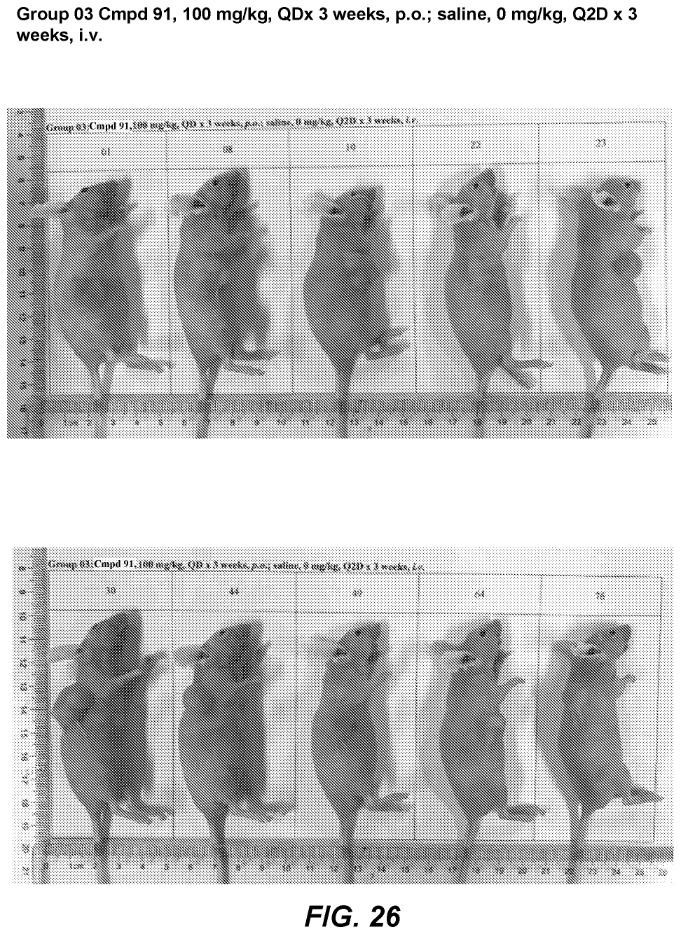

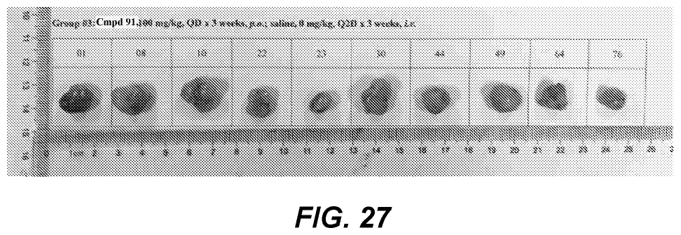

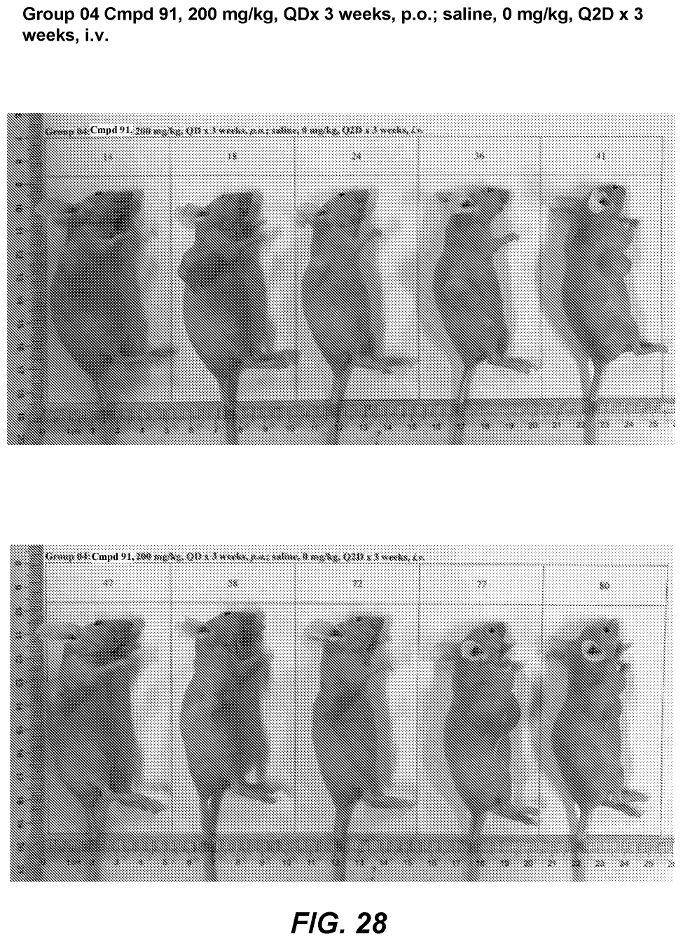

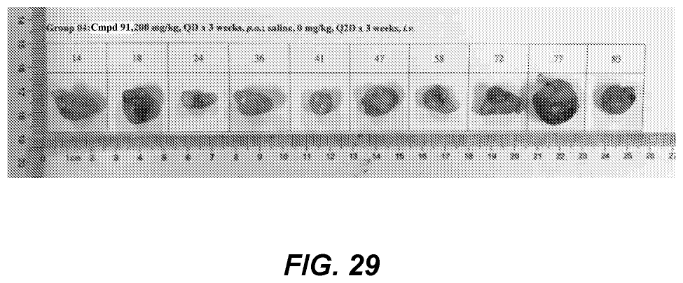

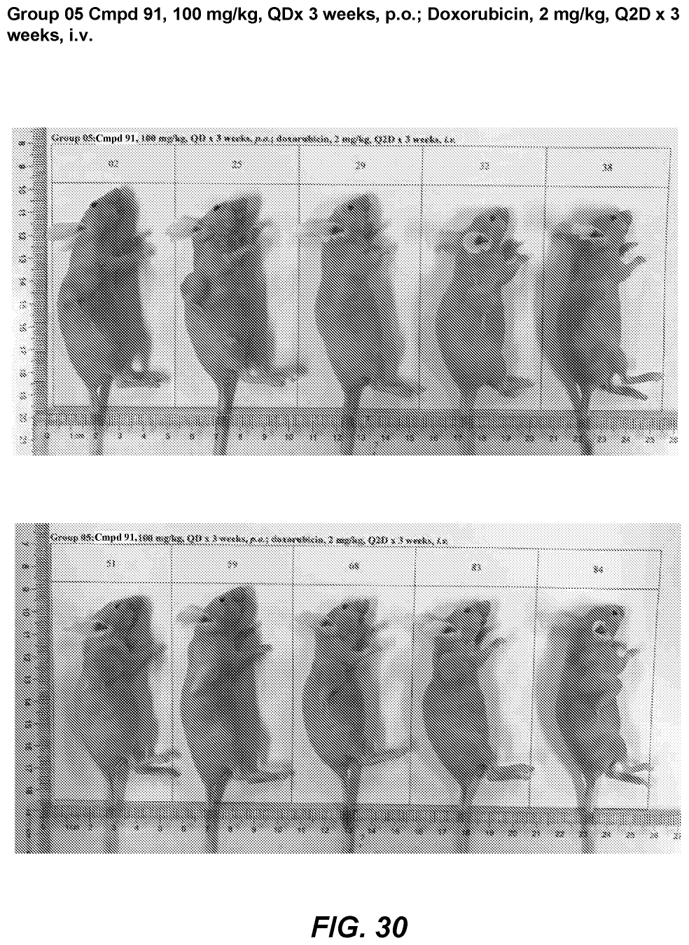

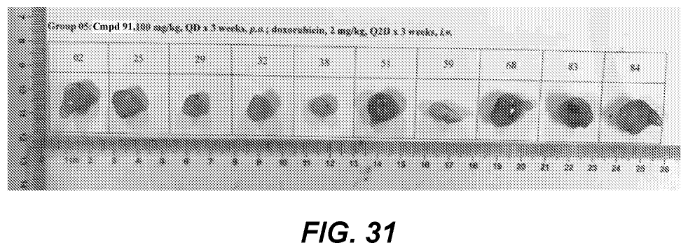

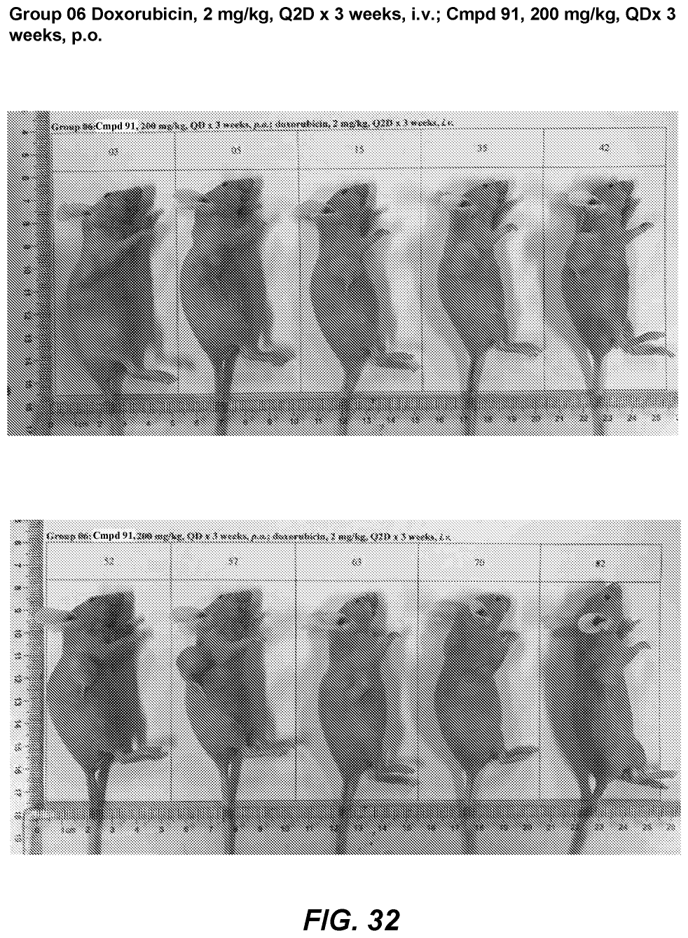

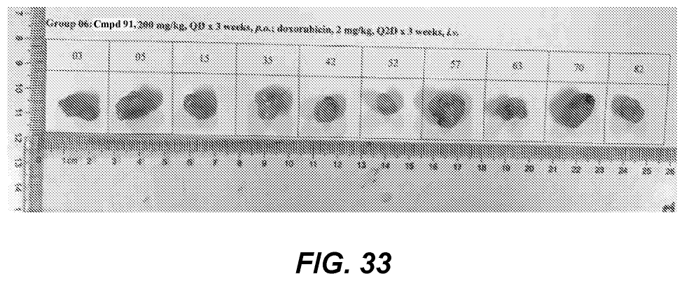

The tumor growth curves are shown in FIG. 8-14. FIG. 8 shows the mean tumor growth curves for each group. FIGS. 9-14 show tumor volume for each group. Inhibition for tumor growth and tumor weight are shown in Table 7 and Table 8. Photos of mice are shown in FIG. 22 (Group 1), FIG. 24 (Group 2), FIG. 26 (Group 3) FIG. 28 (Group 4), FIG. 30 (Group 5), and FIG. 32 (Group 6). Photos of removed tumors are shown in FIG. 23 (Group 1), FIG. 25 (Group 2), FIG. 27 (Group 3) FIG. 29 (Group 4), FIG. 31 (Group 5), and FIG. 33 (Group 6).

A549 human lung cancer cells were inoculated in BALB/c nude mice and treatment was initiated when tumors reached a mean volume of approximately 100 mm³. In this study, only Group 6, treated with Compound 91 at 200 mg/kg and Doxorubicin at 2 mg/kg, demonstrated significant anti-tumor efficacy (TGI: 29%, p<0.05) compared to vehicle control group. Although no statistically significant inhibition of tumor growth was observed, treatment with Compound 91(100 mg/kg) or Doxorubicin (2 mg/kg) alone and in combination resulted in a decrease in the mean tumor volume. For Group 4, treated with Compound 91 at 200 mg/kg and saline, there were 9 tumors (TV<970 mm³) with mean tumor volume of 754.25 mm³ and 1 tumor (2526.23 mm³) at termination. No statistically significant decrease in tumor weight was observed for all treatment groups compared to vehicle control group.

### Embodiments

The following embodiments are included within the scope of this disclosure.

1. A method for treating cancer in a patient in need thereof, the method comprising administering to the patient a therapeutically effective amount of the following therapeutic agents:
i) a PKM2 activator, or a stereoisomer, pharmaceutically acceptable salt, tautomer or prodrug thereof; and
ii) an anti-cancer drug having a mechanism of action that increases production of reactive oxygen species in cancer cells upon administration to the patient, or a stereoisomer, pharmaceutically acceptable salt, tautomer or prodrug thereof.

2. The method of embodiment 1, wherein the PKM2 activator has the following structure (I):

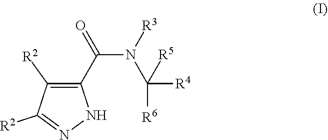

or a stereoisomer, pharmaceutically acceptable salt, tautomer or prodrug thereof, wherein:

R¹ is cycloalkyl, haloalkyl, halo, nitrile or amino;

R² is H or halo;

R³ is alkyl, alkoxyalkyl, cycloalkoxyalkyl, cycloalkyl, cycloalkylalkyl, heterocyclyl, heterocyclylalkyl, heteroaryl, heteroarylalkyl or aralkyl

R⁴ is aryl or heteroaryl

R⁵ and R⁶ are each independently H or alkyl.

3. The method of any one of embodiments 1 or 2, wherein the

PKM2 activator has the following structure (Ia):

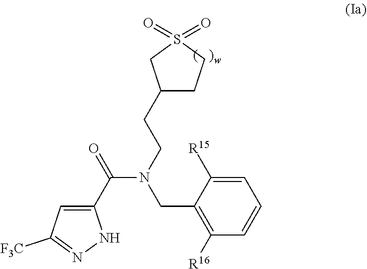

or a stereoisomer, pharmaceutically acceptable salt, tautomer or prodrug thereof, wherein:

R¹⁵ is halo;

R¹⁶ is H or NH₂; and

w is 1 or 2.

4. The method of any one of embodiments 1 or 2, wherein the PKM2 activator has the following structure (Ib):

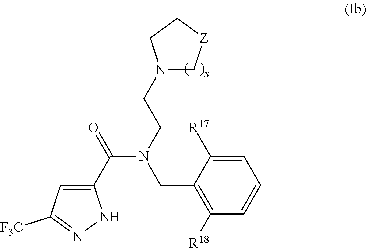

or a stereoisomer, pharmaceutically acceptable salt, tautomer or prodrug thereof, wherein:

R¹⁷ is halo;

R¹⁸ is H or NH₂;

Z is CH₂, O, NH, NR¹⁹, CHR²⁰ or CF₂;

R¹⁹ is alkyl;

R²⁰ is alkoxy, hydroxyl or halo; and

x is 0, 1, 2 or 3.

5. The method of any one of embodiments 1 or 2, wherein the PKM2 activator has the following structure (Ic):

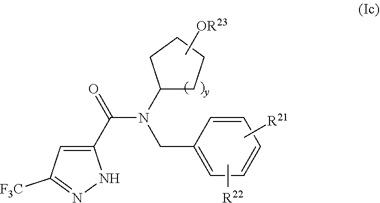

or a stereoisomer, pharmaceutically acceptable salt, tautomer or prodrug thereof, wherein:

R²¹ and R²² are each independently H or halo;

R²³ is H or alkyl; and

y is 1 or 2.

6. The method of any one of embodiments 1-5, wherein the PKM2 activator is selected from Table 1.

7. The method embodiment 6, wherein the PKM2 activator has one of the following structures:

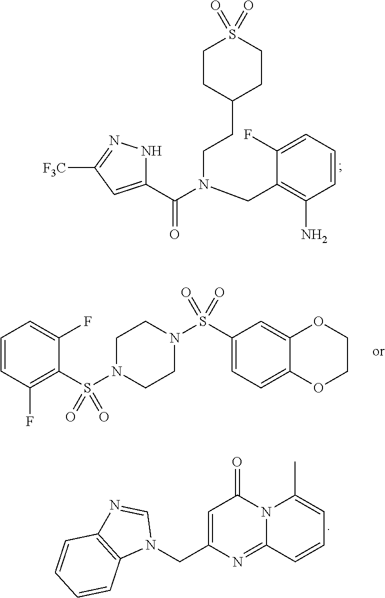 [SMILES: Nc1cccc(F)c1CN(CCC1CCS(=O)(=O)CC1)C(=O)c1cc(C(F)(F)F)n[nH]1 ; O=S(=O)(c1ccc2c(c1)OCCO2)N1CCN(S(=O)(=O)c2c(F)cccc2F)CC1 ; Cc1cccc2nc(Cn3cnc4ccccc43)cc(=O)n12]

8. The method of embodiments 7, wherein the PKM2 activator has the following structure:

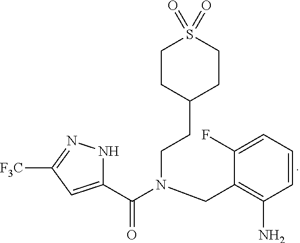 [SMILES: Nc1cccc(F)c1CN(CCC1CCS(=O)(=O)CC1)C(=O)c1cc(C(F)(F)F)n[nH]1]

9. The method of any one of embodiments 1-8, wherein the PKM2 activator selectively lowers glutathione levels in cancer cells.

10. The method of any one of embodiments 1-9, wherein the anti-cancer drug is selected from the group consisting of anthracyclines, anthracenediones, proteasome inhibitors, kinase inhibitors, and HSP90 inhibitors.

11. The method of any one of embodiments 1-10, wherein the anti-cancer drug is an anthracycline.

12. The method of embodiment 11, wherein the anti-cancer drug is selected from the group consisting of doxorubicin and daunorubicin.

13. The method of any one of embodiments 1-10, wherein the anti-cancer drug is an anthracenedione.

14. The method of embodiment 13, wherein the anthracenedione is mitoxantrone.

15. The method of any one of embodiments 1-10, wherein the anti-cancer drug is a proteasome inhibitor.

16. The method of embodiment 15, wherein the anti-cancer drug is bortezomib.

17. The method of any one of embodiments 1-10, wherein the anti-cancer drug is a kinase inhibitor.

18. The method of embodiments 17, wherein the anti-cancer drug is sorafenib.

19. The method of any one of embodiments 1-10, wherein the anti-cancer drug is a HSP90 inhibitor.

20. The method of embodiment 19, wherein the anti-cancer drug is retaspimycin hydrochloride (IPI-504).

21. The method of any one of embodiments 1-20, wherein the PKM2 activator is administered to the patient prior to administration of the anti-cancer drug.

22. The method of embodiment 21, wherein the anti-cancer drug is administered within about 24 to 48 hours after administration of the PKM2 activator.

23. The method of any one of embodiments 1-20, wherein the anti-cancer drug is administered to the patient prior to administration of the PKM2 activator.

24. The method of any one of embodiments 1-23, wherein the cancer is a hematologic cancer.

25. The method of embodiment 24, wherein the hematologic cancer is selected from acute myelogenous leukemia (AML), multiple myeloma, follicular lymphoma, acute lymphoblastic leukemia (ALL), chronic lymphocytic leukemia (CLL) and non-Hodgkin's lymphoma.

26. A method for treatment of cancer in a patient in need thereof, the method comprising reducing glutathione levels in cancer cells of the patient and administering to the patient an anti-cancer drug having a mechanism of action that increases production of reactive oxygen species in cancer cells upon administration to the patient.

27. The method of embodiment 26, wherein reducing glutathione levels in cancer cells of the patient comprises administering a therapeutic agent to the patient, wherein the therapeutic agent reduces glutathione levels in cancer cells of the patient.

28. The method of embodiment 27, wherein the therapeutic agent is a PKM2 activator.

29. The method of embodiment 28, wherein the PKM2 activator is as defined in any one of embodiments 2-9.

30. The method of any one of embodiments 26-29, wherein the anti-cancer drug is as defined in any one of embodiments 10-20.

31. The method of any one of embodiments 26-30, wherein the cancer is as defined in any one of embodiments 24 or 25.

32. A kit comprising a PKM2 activator, an anti-cancer drug having a mechanism of action that increases production of reactive oxygen species in cancer cells upon administration to the patient, and instructions for administering the PKM2 activator and the anti-cancer drug to a patient in need of treatment of cancer.

33. The kit of embodiment 32, wherein the PKM2 activator is as defined in any one of embodiments 2-9.

34. The kit of any one of embodiments 32 or 33, wherein the anti-cancer drug is as defined in any one of embodiments 10-20.

35. The kit of any one of embodiments 32-34, wherein the anti-cancer cancer is as defined in any one of embodiments 24 or 25.

36. A pharmaceutical composition comprising a pharmaceutically acceptable carrier or excipient, a PKM2 activator and an anti-cancer drug having a mechanism of action that increases production of reactive oxygen species in cancer cells upon administration to the patient.

All of the U.S. patents, U.S. patent application publications, U.S. patent applications, foreign patents, foreign patent applications and non-patent publications referred to in this specification are incorporated herein by reference in their entirety to the extent not inconsistent with the present description. U.S. Provisional Patent Application No. 62/572,237, filed October 13, 2017, to which the present application claims priority, is hereby incorporated herein by reference in its entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for treating cancer in a patient in need thereof, the method comprising administering to the patient a therapeutically effective amount of the following therapeutic agents:
i) a PKM2 activator, or a stereoisomer, pharmaceutically acceptable salt, tautomer or prodrug thereof; and
ii) an anti-cancer drug having a mechanism of action that increases production of reactive oxygen species in cancer cells upon administration to the patient, or a stereoisomer, pharmaceutically acceptable salt, tautomer or prodrug thereof

2. The method of claim 1, wherein the PKM2 activator has the following structure (I): 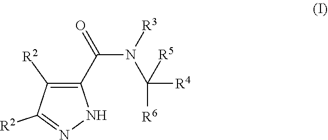
or a stereoisomer, pharmaceutically acceptable salt, tautomer or prodrug thereof, wherein:
R¹ is cycloalkyl, haloalkyl, halo, nitrile or amino;
R² is H or halo;
R³ is alkyl, alkoxyalkyl, cycloalkoxyalkyl, cycloalkyl, cycloalkylalkyl, heterocyclyl, heterocyclylalkyl, heteroaryl, heteroarylalkyl or aralkyl
R⁴ is aryl or heteroaryl
R⁵ and R⁶ are each independently H or alkyl.

3. The method of claim 1, wherein the PKM2 activator has the following structure (Ia): 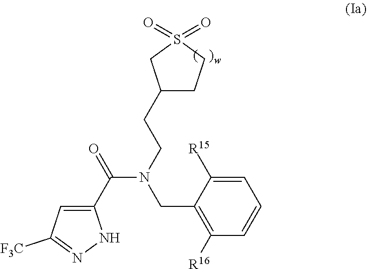
or a stereoisomer, pharmaceutically acceptable salt, tautomer or prodrug thereof,
wherein:
R¹⁵ is halo;
R¹⁶ is H or NH₂; and
w is 1 or 2.

4. The method of claim 1, wherein the PKM2 activator is selected from Table 1.

5. The method claim 4, wherein the PKM2 activator has the following structure:

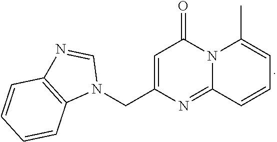 [SMILES: Cc1cccc2nc(Cn3cnc4ccccc43)cc(=O)n12]

6. The method of claims 4, wherein the PKM2 activator has the following structure:

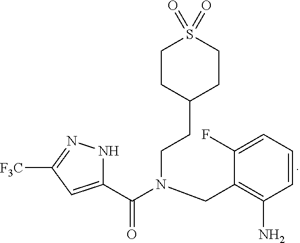 [SMILES: Nc1cccc(F)c1CN(CCC1CCS(=O)(=O)CC1)C(=O)c1cc(C(F)(F)F)n[nH]1]

7. The method claim 4, wherein the PKM2 activator has the following structure:

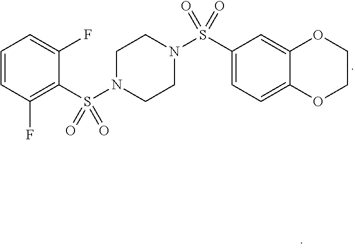 [SMILES: O=S(=O)(c1ccc2c(c1)OCCO2)N1CCN(S(=O)(=O)c2c(F)cccc2F)CC1]

8. The method of claim 1, wherein the anti-cancer drug is an anthracycline.

9. The method of claim 8, wherein the anti-cancer drug is selected from the group consisting of doxorubicin and daunorubicin.

10. The method of claim 1, wherein the anti-cancer drug is a proteasome inhibitor.

11. The method of claim 10, wherein the anti-cancer drug is bortezomib.

12. The method of claim 1, wherein the anti-cancer drug is a kinase inhibitor.

13. The method of claims 12, wherein the anti-cancer drug is sorafenib.

14. The method of claim 1, wherein the anti-cancer drug is a HSP90 inhibitor.

15. The method of claim 14, wherein the anti-cancer drug is retaspimycin hydrochloride (IPI-504).

16. The method of claim 1, wherein the cancer is a hematologic cancer.

17. The method of claim 16, wherein the hematologic cancer is selected from acute myelogenous leukemia (AML), multiple myeloma, follicular lymphoma, acute lymphoblastic leukemia (ALL), chronic lymphocytic leukemia (CLL) and non-Hodgkin's lymphoma.

18. A method for treatment of cancer in a patient in need thereof, the method comprising reducing glutathione levels in cancer cells of the patient and administering to the patient an anti-cancer drug having a mechanism of action that increases production of reactive oxygen species in cancer cells upon administration to the patient.

19. The method of claim 18, wherein reducing glutathione levels in cancer cells of the patient comprises administering a PKM2 activator to the patient, wherein the PKM2 activator reduces glutathione levels in cancer cells of the patient, wherein:
(a) the PKM2 activator has the following structure (I): 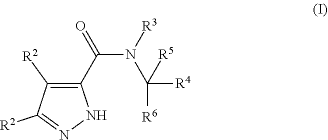
or a stereoisomer, pharmaceutically acceptable salt, tautomer or prodrug thereof, wherein:
R¹ is cycloalkyl, haloalkyl, halo, nitrile or amino;
R² is H or halo;
R³ is alkyl, alkoxyalkyl, cycloalkoxyalkyl, cycloalkyl, cycloalkylalkyl, heterocyclyl, heterocyclylalkyl, heteroaryl, heteroarylalkyl or aralkyl
R⁴ is aryl or heteroaryl
R⁵ and R⁶ are each independently H or alkyl;
(b) the anti-cancer drug is a proteasome inhibitor, a kinase inhibitor, or a HSP90 inhibitor;
(c) the cancer is a hematologic cancer; or
(d) a combination thereof.

20. A kit comprising a PKM2 activator, an anti-cancer drug having a mechanism of action that increases production of reactive oxygen species in cancer cells upon administration to the patient, and instructions for administering the PKM2 activator and the anti-cancer drug to a patient in need of treatment of cancer.
